(19) United States
(12) Patent Application Publication
(10) Pub. No.: US 2025/0134930 A1
(43) Pub. Date: May 1, 2025

(54) REGULATION OF CELLS AND ORGANISMS

(71) Applicants: Victor TETS, New York, NY (US); Georgy TETS, New York, NY (US)

(72) Inventors: Victor TETS, New York, NY (US); Georgy TETS, New York, NY (US)

(21) Appl. No.: 18/926,059

(22) Filed: Oct. 24, 2024

Related U.S. Application Data
(63) Continuation of application No. 18/902,315, filed on Sep. 30, 2024.
Application No. 18/902,315 is a continuation-in-part of application No. 18/285,643, filed on Oct. 4, 2023.
(60) Provisional application No. 63/170,885, filed on Apr. 5, 2021.

(51) Int. Cl. A61K 35/17; A61K 35/15; A61K 35/19; A61P 35/00; C12N 9/22

(52) U.S. Cl. CPC A61K 35/17; A61K 35/15; A61K 35/19; A61P 35/00; C12N 9/22

(57) ABSTRACT

The invention relates to medicine, biology, veterinary, pharmacology diagnostics, agriculture, ecology, meteorology, seismology, construction biotechnology, biomanufacturing and provided herein are products and methods for managing cells behavior, memory of cells and erasure of cell memory. The present invention describes products and methods that, unlike the known ones, make it possible to control the properties of cells and organisms without the use of mutagens and/or the special introduction of genes and/or use of specific gene tools and/or changing its environmental conditions.

### FIELD OF THE INVENTION

The invention relates to medicine, biology, veterinary, pharmacology diagnostics, agriculture, ecology, meteorology, seismology, construction, biotechnology, biomanufacturing and provided herein are products and methods for managing cells behavior, memory of cells and erasure of cell memory.

### BACKGROUND OF THE INVENTION

A known method to change genetic information in cells is by introducing new genes. In this case, genes are introduced into the cell by various ways: transformation, transduction and other techniques known in the art. The introduced genes either carry new information or turn off the existing genes.

There is a known method for changing the properties of a cell by editing the genome, when molecules are introduced into the cell that can artificially change the structure of the genome, cutting out and sewing in the genes (Spicer et al 2018).

The present invention describes products and methods that, unlike the known ones, make it possible to control the properties of cells and organisms without the use of mutagens and/or the special introduction of genes and/or use of specific gene editing tools and/or changing its environmental conditions.

### Definitions

Inactivation-destruction; inactivation; cleavage; decrease of the number; inhibition of activity; that are done in vitro, in vivo and/or ex vivo and in any materials.

Alteration-modification; alteration of activity; alteration of structure; alteration of conformation; alteration of nucleic acid components; alteration of binding or association with other molecules i.e. metals, protein, lipid and other nucleic/non-nucleic acids components; qualitative and/or quantitative alterations; alteration of signal generation, reception, transduction, modification; increase of the number; disposition; alteration of activity; restoration after alteration; incomplete restoration after alteration; alteration of production; alteration of their secretion outside the cells; magnetization; that are done in vitro, in vivo and/or ex vivo and in any materials.

Cut-D cells One-time treatment with DNA inactivating product.

Cut-R cells-One-time treatment with RNA inactivating product.

Cut-DR cells or “Drunk cells” One-time treatment with DNA+RNA inactivating products.

Zero-D cells after 2 and more cycles with DNA inactivating products with placing of cells between treatments with DNA inactivating products to the minimal growth conditions (ZD).

Zero-R cells after 2 and more cycles with RNA inactivating products with placing of cells between treatments with RNA inactivating products to the minimal growth conditions (ZR).

Zero-DR Cells after 2 and more cycles with DNA and RNA inactivating products with placing of cells between treatments with DNA and RNA inactivating products to the minimal growth conditions (Z0).

Y-D cells—2 and more cycles with DNA inactivating products with placing of cells between treatments with DNA inactivating products to the same and/or nutritional rich growth conditions.

Y-R—2 and more cycles with RNA inactivating products with placing of cells between treatments with RNA inactivating products to the same and/or nutritional rich growth conditions.

Y-DR-2 and more cycles with DNA and RNA inactivating products with placing of cells between treatments with DNA and RNA inactivating products to the same and/or nutritional rich growth conditions.

NAMACS and NAMACS-ANA—nucleic acid molecule(s) associated with cell surfaces and/or other nucleic acids associated with these surface-associated nucleic acids.

TEZR is a nucleic acid molecule(s) associated with cell surfaces and/or other nucleic acids associated with these surface-associated nucleic acids, capable of recognizing biological, chemical, mechanical and physical factors and generating cell responses.

TEZR can be specific to different cell types and have a length from 2 to 1,000,000 nucleotides.

Microorganisms: bacteria, archaea, fungi, protists, unicellular eukaryotes, unicellular algae, viruses.

Managing: control, regulation, sensing, modulation, alteration, manipulation, management, adjustment.

### SUMMARY OF THE INVENTION

In some embodiments products can destroy and/or inactivate NAMACS and NAMACS-ANA, reverse transcriptase inhibitors, recombinase inhibitors including, protease inhibitors, integrase inhibitors, recombinases as well as cells, organoids, tissues formed following the treatment with these products.

In one embodiment products to be used in medicine, veterinary, ecology, meteorology, seismology agriculture, construction, biotechnology, biomanufacturing for managing functions of procaryotes, eukaryotes including mammalians, plants, fungi, animals, organoids, tissues, embryos, organs, single-cellular, and multicellular organisms.

In some embodiments the products are used for managing relationship to physical, chemical, mechanical and biological factors.

In some embodiments the products can violate signal generation and/or transmission in inside cells and/or outside cells.

In some embodiments the products are used for the diagnosis, treatment and prevention of diseases caused by protozoa, bacteria, fungi and viruses.

In some embodiments products are used for managing the recombination of DNA and/or switching on and/or off of the genes.

In some embodiments products are used for managing the formation of spores of bacteria and fungi.

In some embodiments products are used for managing the synthesis of DNA and/or RNA and/or proteins.

In some embodiments products are used for managing post-synthetic modification of nucleic acids and/or proteins; DNA methylation.

In some embodiments products are used for managing the spread of cells; and the resettlement of bacterial biofilms.

In some embodiments products are used for managing the spread of metastases.

In some embodiments products are used for managing of cell properties by turning cells to “Cut” (including “Drunk cell”), “Zero”, “Y” states.

In some embodiments regulation of cells properties is by the inactivation TEZRs.

In one embodiment of any of the methods of the invention, the subject is human.

In some embodiments products are used for managing single-strain DNA, double-strain DNA, single-strain RNA, double-strain RNA, DNA-RNA hybrid, Doble-helical DNA, Pauling triplex, G-quadruplex.

In some embodiments products are used for managing organoids including mitochondria and plastids.

In some embodiments TEZRs are on the surface or within membrane vesicles.

In some embodiments products are used for managing process that at least partially regulated by type IV secretion.

In some embodiments s, formation of TEZRs is done by management of type IV secretion In some embodiments products are used for managing the participation of reverse transcription, RNA dependent RNA synthesis, and the formation of nucleic acid molecule(s) associated with the surface of cells and/or associated with them that can trigger formation of the isoforms of proteins and nucleic acids with altered properties.

In some embodiments qualitative and/or quantitative alterations of TEZRs is done within extracellular vesicles.

In some embodiments products are used for managing the work of cell surface receptors with a non-limiting examples of protein receptors.

In some embodiments NAMACS and/or NAMACS-ANA and/or TEZRs are artificial.

In some embodiments products are used for managing the work of cell protein kinase.

In some embodiments products are used for managing signal transduction in mammals and microbial communities.

In some embodiments products are used for managing gene transfer by viruses in mammals and microbial communities.

In some embodiments products are used for managing cells activity within any of the component of microbiota-gut-brain axis.

In some embodiments products are used for managing bacterial colonization and migration.

In some embodiments products are used for managing mutagenesis and/or cell adhesion to the substrate and/or rate of cells division, and/or limit of cell divisions.

In some embodiments products are used for managing of DNA recombination.

In some embodiments products are used for managing interaction cells and extracellular molecules proteins and/or DNA and/or RNA with prion-like domains of proteins.

In some embodiments products are used for managing process that are associated with reverse transcriptase, of retroelements, group II introns, CRISPR-Cas systems, diversity-generating retroelements, Abi-related RTs, retrons, multicopy single-stranded DNA (msDNA), splicing process.

In some embodiments NAMACS and/or NAMACS-ANA and/or TEZRs are linked to the receptors with proteomic structure.

In some embodiments products are used for managing microbial dormancy and persistence.

In some embodiments products are used for the increase of cell survival at conditions when untreated cells will die.

In some embodiments products are used for managing the resurrection.

In some embodiments products are used for managing the arrest or increase of apoptosis and/or necrosis and/or necroptosis and/or other types of cell deaths.

In some embodiments products are used for managing in cell to cell transport of different genes that can be coded in DNA or RNA molecules and activity of cell reverse transcriptase(s) by which RNA molecules can be transformed in DNA.

In some embodiments products are used for managing targeted cell delivery.

In some embodiments products are used for managing nlrp3 inflammasome, caspase 1 work and pathway, NF-kB pathway.

In some embodiments products are used for managing of prokaryote-prokaryote prokaryote-eukaryote and eukaryote-eukaryote interactions.

In some embodiments negative impact of the outer environment is ameliorated by wearing clothing that modulates the effects of geomagnetic filed on NAMACS and/or NAMACS-ANA and/or TEZRs In some embodiments products are used for managing weather-dependence. In some embodiments products as vaccina against cells NAMACS and/or NAMACS-ANA and/or TEZRs and/or DNase and/or RNase are used for the treatment of diseases and life prolongation.

In some embodiments nucleoside and non-nucleoside inhibitors of reverse transcriptase are used alone or in combination with nucleases and/or antibiotics to treat bacterial infections.

In some embodiments qualitative and/or quantitative alterations of NAMACS and/or NAMACS-ANA and/or TEZRs are used for managing functions of procaryotes, eukaryotes including mammalians, plants, fungi, animals, cells, organoids, tissues, embryos, organs, single-cellular, and multicellular organisms with antibodies, mini antibodies, single-domain antibodies (nanobodies), antibodies with nuclease activity (abzymes), antibodies conjugated with nucleases, and other antibody variants, and/or nucleases endonucleases and/or restrictases, and/or exonuclease, with a non-limiting examples of DNase I, DNase X, DNase γ, DNase1L1, DNase1L2, DNase 1L3, DNase II (e.g., DNase 11a, DNase IIβ), caspase-activated DNase (CAD), endonuclease G (ENDOG), AbaSI, AccI, Acc65I, AciI, AciI, AcuI, AfeI, AfIII, AfIIII, AgeI, AhdI, AleI-v2, AluI, AlwI, AlwNI, ApaI, ApaLI,ApoI, AscI, AseI, AsiSI, AvaI, AvaII, AvrII, BaeGI, BaeI, BamHI, BanI, BanII, BbsI, BbvCI, BbvI, BccI, BceAI, BcgI, BciVI, BcII BfaI BgII BgIII BlpI, BmgBI, BmrI, BmtI, BpmI, BpuEI, Bpu10I, BsaAI, BsaBI, BsaHI, BsaI-HF, BsaJI, BsaWI, BsaXI, BseRI, BseYI, BsgI, BsiEI, BsiHKAI, BsiWI, BsII, BsmAI, BcoDI, BsmBI-v2, BsmFI, BsmI, BspCNI, BspEI, BspHI, Bsp1286I, BspMI BfuAI, BsrBI, BsrDI, BsrFI-v2, BsrGI, BsrI, BssHII, BssSI-v2, BstAPI, BstBI, BstEII, BstNI, BstUI, BstXI, BstYI, BstZ17I, Bsu36I, BtgI, BtgZI, BtsCI, BtsIMutI, Btsl-v2, Cac81, Clal BspDI, CspCI, CviAII, CviKI-1, CviQI, DdeI, DpnI, DraI, DraIII, DrdI, EaeI, EagI, EarI, EciI, Eco53kI, EcoNI, EcoO1091, EcoP151, EcoRI, EcoRV, Esp3I, FatI, FauI, Fnu4HI, FokI, FseI, FspEI, FspI, HaelI, HaeIII, HgaI, HhaI, HincII, HindIII, HinfI, HinP1I, HpaI, HphI, HpyAV, HpyCH4III, HpyCH4IV, HpyCH4V, Hpy1881, Hpy991, Hpy166II, Hpy188III, I-CeuI, I-SceI, KasI, KpnI, LpnPI, MboI, MbolI, MfeI, MluCI, MlyI, MmeI, MnlI, MscI, MseI, MslI, MspA1I, MspI HpalI, MspJI, MwoI, NaeI, NarI, Nb.BbvCI, Nb.BsmI Nb.BsrDI, Nb.BssSI, Nb.BtsI, NciI, NcoI NcoI-HF, NdeI, NgoMIV, NheI NheI-HF, NlaIII, NlaIV, NmeAllI, NotI NotI-HF, NruI NruI-HF, NsiI NsiI-HF, NspI, Nt.AlwI, Nt.BbvCI, Nt.BsmAI, Nt.BspQI, Nt.BstNBI, Nt.CviPII, PacI, PaqCI, PciI, PfIMI, PI-PspI, PI-SceI, PleI, PluTI, PmeI, PmlI, PpuMI, PshAI, Psil-v2, PspGI, PspOMI, PspXI, Pstl Pstl-HF, PvuI PvuI-HF, PvuII PvuII-HF, RsaI, RsrII, SacI SacI-HF, SaclI, SalI SalI-HF, SapI BspQI, Sau961, SbfI SbfI-HF, ScaI-HF, ScrFI, SexAI, SfaNI, SfcI, SfiI, SfoI, SgrAI, SmaI, SmlI, SnaBI, SpeI SpeI-HF, SphI SphI-HF, SrfI, SspI SspI-HF, StuI, StyD4I, StyI-HF, SwaI, TaqI-v2, TfiI, Tsel ApeKI, Tsp451, TspRI, Tth111I PfIFI, XbaI, XcmI, Xhol PaeR7I, Xmal TspMI, XmnI, ZraI, granzyme B (GZMB), Exonuclease I, Exonuclease V, Exonuclease VII, Exonuclease III, RNaself, RNase III, RNase H1, Exonuclease I, lambda exonuclease, REC BCD nuclease, REC J nuclease, T6 gene exonuclease, combination of thereof, and mutants or derivatives thereof], phosphodiesterase I, lactoferrin, acetylcholinesterase, engineered nucleases, transferases (i.e. methylase), intercalators, different molecules as adapters, mitomycin C, bleomycin, metals, oligonucleotides, polysaccharides, aptomers, protector from nucleases, reverse transcriptase inhibitors and/or salts of orotic acid, and/or ribavirin and/or acyclovir, and/or compound VTL and/or recombinases, protease inhibitors and/or integrase inhibitors, ultrasound and other wave-methods, viruses and their components.

In some embodiments alteration of NAMACS and/or NAMACS-ANA and/or TEZRs include destruction; inactivation; alteration of activity; alteration of structure; alteration of conformation; alteration of nucleic acid components; alteration of binding or association with other molecules i.e. metals, protein, lipid and other nucleic/non-nucleic acids components; qualitative and/or quantitative alterations, alteration of signal generation, reception, transduction, modification; increase or decrease of the number; disposition; restoration after alteration; alteration of production; alteration of their secretion outside the cells; magnetization; that are done in vitro, in vivo and/or ex vivo and in any materials.

In some embodiments products for managing functioning of cells, tissues, organs, organisms, plants and/or plant seeds can be used prior, together and/or after with reverse transcriptase inhibitors and/or recombinase inhibitors, and/or protease inhibitors and/or integrase inhibitors and/or proteases and/or salts of orotic acid, and/or ribavirin and/or acyclovir, antibodies and/or compound VTL.

In some embodiments for managing of plants characteristics treatment with integrase inhibitors prior, together or following the treatment by products are used during the soak.

In some embodiments water, soil, films that contact with seeds or plants or their parts contain and/or are impregnated with nucleases, transferases (i.e. methylase), intercalators, and/or different molecules binding to them of adapters, mitomycin C, bleomycin, metals, reverse transcriptase inhibitors of nucleoside and/or non-nucleoside reverse transcriptase inhibitors and/or salts of orotic acid, and/or ribavirin and/or acyclovir, recombinases and protease inhibitors and/or integrase inhibitors.

In some embodiments treatment of cells and/or their components) with products alter TLRs activity.

In some embodiments treatment of cells and/or their components with products modulate MyD88-STAT3 or MyD88-NF-kB pathways.

In some embodiments, TezRs are restored with aptamers.

In some embodiments, wherein labware (tips, pipettes, dishes, plates, tubes), disposables, liquids, (i.e. PBS, water), nutrient media, contain products to generate cells with new characteristics.

In one embodiment microbial or eukaryotic cells in “Cut” including “Drunk cell”, “Zero”, “Y” states are transplanted to the individual including the same individual from whom these cells were collected with non-altered and/or reprogrammed and/or erased memory.

In one embodiment wherein, eukaryotic cells (i.e. stem cells, hematopoietic stem cell, fibroblasts, endothelial cells, renal cells, immune cells, blood cells) are treated by products to be turned to “Cut” including “drunk cell”, “Zero”, “Y” states prior of being transplanted to the recipient.

In one embodiment the cells in the states as “Cut” including “drunk cell”, “Zero”, “Y” states are used to transfer cells to/from a pluripotent state are used for the reparation and/or regeneration of tissues, organs, part of the body of animals, plants.

In some embodiments, treatment of prevention of diseases is caused by the destruction of TezRs outside or inside the cells.

In some embodiments wherein procaryotic and/or eucaryotic cells, that produce factors that inactivate DNA and/or RNA including representatives of Bacillaceae (i.e. Bacillus spp), Enterobacteriaceae (i.e. E. coli, Salmonells spp., Klebsiella spp.), Pseudomonadaceae, Lactococcoceae, Clostridiaceae families and fungi Aspergillus spp. are added to the soil or water for irrigation.

In some embodiments the products as enzymes which have a nuclease activity is DNase I, various mutants weakening actin-binding may be used. Specific non-limiting examples of residues in wild-type recombinant human DNase I that can be mutated include, e.g., Gln-9, Glu-13, Thr-14, His-44, Asp-53, Tyr-65, Val-66, Val-67, Glu-69, Asn-74, and Ala-114. In various embodiments, the Ala-114 mutation is used. For example, in human DNase I hyperactive mutant comprising the sequence of the Ala-114 residue is mutated. Complementary residues in other DNases may also be mutated. Specific non-limiting examples of mutations in wild-type human recombinant DNAse I include H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N: T46S, D53R: Y65A, D53R: E69R, H44A: D53R: Y65A, H44A: Y65A: E69R, H64N: V66S, H64N: V66T, Y65N: V67S, Y65N: V67T, V66N: S68T, V67N: E69S, V67N: E69T, S68N: P70S, S68N: P70T, S94N: Y96S, S94N: Y96T. Various DNase mutants for increasing DNase activity may be used. Specific non-limiting examples of mutations in wild-type human recombinant DNAse I include, e.g., Gln-9, Glu-13, Thr-14, His-44, Asp-53, Tyr-65, Val-66, Val-67, Glu-69, Asn-74, and Ala-114. Specific non-limiting examples of mutations for increasing the activity of wild-type human recombinant DNase I include Q9R, E13R, E13K, T14R, T14K, H44R, H44K, N74K, and A114F. For example, a combination of the Q9R, E13R, N74K and A114F mutations may be used.

In some embodiments for cells managing for diagnosis, treatment and prevention of diseases and antibiotics resistance development as well as antibiotics resistance overcoming reverse transcriptase inhibitors and substances of the pyrimidine series, namely 2-chloro-5-phenyl-5H-pyrimido[5′,4′:5,6] pyrano[2,3-d]pyrimidine-4-ol derivatives are used.

In some embodiments products can be used in combination with drugs, formulations, procedures, medical interventions with a non-limiting examples of anticancer (with a non-limiting examples of chemotherapy, immunotherapy [PD-1, PD-L1, OX-40, CTLA-4 inhibitors], gene therapy, CAR-T, radiotherapy, antimicrobial, antiviral, antipain, antistress, antiaging, regenerative, hormones, stimulators, antibodies, antipyretics used to the prophylactic and treatment of the diseases and conditions of digestive; cardiovascular, central nervous, musculoskeletal, traumas otolaryngology, ophthalmology, respiratory, endocrine, reproductive, urinary, obstetrician and gynecological, skin systems; immune and autoimmune diseases, immunosuppressive drugs (with a non-limiting examples of TNF blockers), antibiotic therapy, antipain medicine, siRNA, siDNA, oncolytic viruses, surgery, nutrition, pre-neoplastic and/or neoplastic processes.

In some embodiments, for prokaryote or eukaryote managing antibodies are used.

In some embodiments turning cells to “Cut”, “Zero”, “Y” states may lead to the dysfunction of receptors with a non-limiting examples of tyrosin-kinase-based receptors such as EGFR, Tumor necrosis factor related apoptosis-inducing ligand, TLRs, Serotonin receptors, CTLA-4, PD-1, and PD-L1, PD-L2, B7 family, VISTA, Tim-3 and LAG-3, TCR, MHC, Gal-9, MHCII, HHLA2, LSECtin, CD80/86, CD5, CD7, CD4, CD3, CD28, TIL, estrogen receptor, progesterone receptor, human epidermal growth factor receptor, VEGF, VEGFR, RYK, GDNF, RET, ERBB, INSR, IGF-1R,IRR, PDGFR, CSF-1R, KIT/SCFR, FLK2/FLT3, FGFR, CCK4, TRKS, TRKB, TRKC, MEN, RON, EPHA, AXL, MER, TYRO, TIE, TEK, DDR, ROS, LTK, ALK, ROR, MUSK, AATYK, RTK INSR group, FGFR group, EGFR group, EPH group, ROR group; and that affect signaling pathway with a non-limiting examples of those associated with WNT, SRC, PI3K, PTEN, AKT, mTOR, PARP, CHK1/2, WEE, and can be used alone or in combination with other drugs targetnig such a receptors with a non-limiting examples of monoclonal antibodies (mAbs) that target the extracellular domain and/or receptor catalytic domains, and that affect aberrant protein phosphorylation.

In some embodiments the use of information, which is recorded in NAMACS and/or NAMACS-ANA and/or TEZRs can be used for the diagnosis, treatment and prevention of neurodegenerative diseases; pain; cardiovascular diseases; diseases of the gastrointestinal tract; diseases of the urinary system; diseases of the musculoskeletal system; injuries; traumas, cancer; blood diseases; migraine and weather-dependent conditions; negative health conditions associated with air travel; conditions associated with poisoning of various nature; receiving doses of radiation; conditions associated with UV exposure; conditions associated with overheating; conditions associated with hypothermia; directions of repair processes for injuries and surgical interventions.

In some embodiments routes of administration of the invention include, e.g., intracerebral, intracerebroventricular, intraparenchymal injections, intrastriatal, intraspinal, parenteral (including subcutaneous, intramuscular, intravenous, intraarterial, inhalation, intradermal, intrathecal, intracisterna magna, epidural and infusion), subarachnoid injection, enteral (e.g., oral), intramuscular, intraperitoneal, transdermal, rectal, nasal, local (including buccal or sublingual), vaginal, intraperitoneal, a local, topical including transdermal, etc.

In some embodiments, DNase and/or RNase delivery to the cells is done by using Lipid Nanoparticle Delivery, Gold nanoconjugated particles, and/or loaded poly (D, L lactide-co-caprolactone) nanocapsules and/or other Nanoparticles and/or, Biohybrid microrobots, microorganisms are used to target the specific cells in mammalians.

In some embodiments, a method for the treatment and prevention of human diseases, by the therapeutic and prophylactic vaccines against NAMACS and/or NAMACS-ANA and/or TEZRs.

In some embodiments the specificity to deliver products is achieved with the delivery of armed antibodies of humanized or chimeric antibodies, antibody fragment targeting the antigen, targeted nanomedicines, peptides, antibody-drug conjugates against TezRs or their components.

In some embodiments the products are used for the treatment of bacterial/HIV-1 co-infection with non-limiting example to be used in patients administering reverse transcriptase inhibitors.

In some embodiments regulation or production, activation, work of NAMACS and/or NAMACS-ANA and/or TEZRs are regulated by genes that are related to retrons with a non-limiting examples of genes: msr, msd and RT (msr-msd-RT).

In some embodiments cells behavior is regulated by products or their mix with aminoglycosides, annamycin, beta-lactams, carbapenem, cephalosporins, carbapenems, chloramphenicol, fluoroquinolones, glycopeptides, lincosamides, lipopeptides, macrolides, monobactams, nitrofurans, oxazolidinones, penicillin, polypeptides, peptide antimicrobial agents, quinolones, sulfonamides, tetracyclines, streptogramins, rifamicin, myxopyronin, azoles, polyenes, 5-fluorocytosin, echinocandins, trimethoprim sulfamethoxazole, nitrofurantoin, urinary anti-infective, lipopeptides, sulfonamides, annamycin's, nitrofurantoin, nitroimidazole, triterpenoids, azoles, echinocandin, nitroimidazole, polyene antibiotics, triterpenoids, peptide antimicrobial agents, bacteriophages, as well as antiseptics and disinfectants (i.e. alcohols, aldehydes, anilids, biguanides, phenols, diamidines, halogen releasing agents, metal derivatives, peroxygens, quaternary ammonium compounds, vapor phase.

In some embodiments inactivation and/or alteration increase and/or decrease and/or modification activity of tumor cells or tumor microenvironment is done with the use cells that migrate to the tumors and/or metastasis (or having a tropism for tumor or tumor environment or capable of engulfing the solid tumors) carrying the genes for synthesis and/or excretion of nucleases with a non-limiting examples of DNase, RNase and their combinations that are delivered straight to tumors and that are administered by different ways with a non-limiting example of p.o, i.v. i.p., intra-tumor etc.

In some embodiments nuclease producing cells are in “Cut”, “Zero”, “Y” states and are used in combination with surgery, local or systemic chemotherapy, immunotherapy, radiotherapy and other targeted therapies.

In some embodiments Bacillaceae (Bacillus spp), Enterobacteriaceae (with a non-limiting examples of E. coli, Salmonella spp., Klebsiella spp.,) Pseudomonadaceae, Bifidobacteriaceae, Clostridiaceae are used.

In some embodiments to increase the release of nucleases within the tumor, lytic phages are used against these bacteria or activation of prophages within bacteria after which bacterial subpopulation producing nucleases die with the release of nucleases.

In some embodiments typing of NAMACS and NAMACS-ANA, TEZRs can be used for the identification of the cells.

In some embodiments determination of the characteristics of NAMACS and/or NAMACS-ANA of bacteria and fungi are used to modulate efficacy of sterilization including pasteurization, estimating and/or predicting of the efficacy of sterilization.

In some embodiments products can manage activity of eukaryotic cells, tissues, organs for the modulation of microorganisms' and/or eukaryotic cells' of the immune cells and/or viruses (including oncolytic) migration towards these cells, tissues and organs with a non-limiting examples with the ability to boost immune response and or kill these cells.

In some embodiments a qualitative or quantitative analysis of NAMACS and/or NAMACS-ANA and/or TEZRs on prokaryotes and eukaryotes can be used as a biomarkers for the drug therapy efficacy

In some embodiments analysis of the presence of NAMACS and NAMACS-ANA, and/or TEZRs and/or DNase and RNase genes, their expression, level and activity of microbial nucleases in cells, tissues, biofluids are used to analyze, predict and modulate bacterial and cellular growth, interactions and sensitivity to antibiotics, immunotherapy, chemotherapy.

In some embodiments therapeutic effect is achieved by colonization of macroorganism by nuclease-producing microorganisms and eukaryotes.

In some embodiments prophylactic and/or treatment of diseases is achieved by the decrease of DNase and/or RNase activity of cells, human tissues, extracellular space, biofluids of nervous tissue, brain, cerebrospinal fluid, including alterations of ion channels, membrane polarization, electrophysiological parameters, neuronal excitability and synaptic plasticity.

In some embodiments products are used to regulate the activity of nervous cells, formation and maintaining of memory

In some embodiments products can modulate mammalian memory.

In some embodiments products are used for the modulation of the memory of “physiological conditions” it a non-limiting examples of pH, temperature, magnetic field, memory, cell memory, taxis, synergism and antagonism, nutrients, oxygen consumption, gas content.

In some embodiments, products are used for regulation memory of antibody-forming cells.

In some embodiments, products can disrupt sense, form and/or transmits and/or transfer signals between molecules, generate a response between cells, group of cells, tissues, organs, organisms.

In some embodiments products usage with/or without of plating cells to a new environment some part of which has to be remembered by the cells leads to the formation of a new and/or altered memory.

In some embodiments plating cells to “Cut”, “Zero”, “Y” state with plating cells to a new conditions results in cells reprogramming and will provide cells with the new properties.

In some embodiments products are used to boost immune cell memory to improve vaccines.

In some embodiments analysis of NAMACS and/or NAMACS-ANA and/or TEZRs including those having non-coding genetic information, is used for diagnostics of age, cell health and disease, origin of cells.

In some embodiments, products make cell more susceptible to reprogramming and, consequently, makes the process of reprogramming quicker and more efficient.

In some embodiments, products for reprogramming of cells can be done together with the alterations and modifications of other chaperons, with a non-limiting example of CAF-1 histone chaperone.

In some embodiments products can to modulate adaptation, chemotaxis, taxis, reflexes of eukaryotes or prokaryotes.

In some embodiments products can enhance cells cognition and spatial memory.

In some embodiments treatment of cells with products and NAMACS and/or NAMACS-ANA and/or TEZRs can increase the efficacy of neurotechnology, computers interface, brain-machine interface, intelligence algorithms, can be used to connect computers to organisms, used for neuronets development.

In some embodiments products are used to regulate fertilization, speed and characteristics of the development of the embryo of fish, birds, other animals, humans.

In some embodiments products are used regulate remote sensing.

In some embodiments products are used for managing epigenetic memory.

In some embodiments products are used for prokaryotic or eukaryotic cells forgetting.

In some embodiments products are used to regulate memorization and/or speed of memorization, and/or long-term and/or short memory formation.

In some embodiments products usage can alter methylation within the promoter regions of tumor suppressor genes causes their silencing, and methylation within the gene itself can induce mutational events.

In some embodiments products usage can modulate bacterial metabolism including metabolism of drugs such as hormones, corticosteroids, anticancer drugs, drugs used for the treatment of infectious diseases, drugs used for the treatment of neurodegenerative disorders.

In some embodiments human diseases are the result of inactivation and/or alteration of TEZRs and/or increase and/or decrease and/or modification their activity of prokaryotic and/or eukaryotic cells.

In some embodiments, process of cells malignization and/or oncogene activation and/or prometastatic genes activation, turning normal cells to malignant, epithelial-mesenchymal transition can be regulated by the alteration of NAMACS and/or NAMACS-ANA and/or TEZRs.

In some embodiments products can make antibiotic resistant bacteria susceptible to antibiotics.

In some embodiments products can be used to modulate NAMACS and/or NAMACS-ANA disease-associated susceptibility genes, include, but are not limited to, ADAR1, MDA5 (IFIH1), RNase H subunits, SamHD1, TREX, TBK1, Optineurin, P62 (sequestosome 1), Progranulin, FUS, VCP, CHMP2B, Profilin-1, Amyloid-β, Tau, α-synuclein, PINK, Parkin, LRRK2, DJ-1, GBA, ATPA13A2, EXOSCIII, TSEN2, TBC1D23, Risk-factor alleles, PLCG2, TREM2, APOE, TOMM40, IL-33, Glucocerebrosidase, Ataxin2, C9orf72, SOD1, and FUS, ABL1 (ABL), ABL2 (ABLL, ARG), AKAP13 (HT31, LBC. BRX), ARAF1, ARHGEF5 (TIM), ATF1, AXL, BCL2, BRAF (BRAF1, RAFB1), BRCA1, BRCA2 (FANCD1), BRIP1, CBL (CBL2), CSF1R (CSF-1, FMS, MCSF), DAPK1 (DAPK), DEK (D6S231E), DUSP6 (MKP3,PYST1), EGF, EGFR (ERBB, ERBB1), ERBB3 (HER3), ERG, ETS1, ETS2, EWSR1 (EWS, ES, PNE), FES (FPS), FGF4 (HSTF1,KFGF), FGFR1, FGFR10P (FOP), FLCN, FOS (c-fos), FRAP1, FUS (TLS), HRAS, GLI1, GLI2, GPC3, HER2 (ERBB2, TKR1, NEU), HGF (SF), IRF4 (LSIRF, MUM1), JUNB, KIT (SCFR), KRAS2 (RASK2), LCK, LCO, MAP3K8 (TPL2, COT, EST), MCF2 (DBL), MDM2, MET (HGFR, RCCP2), MLH type genes, MMD, MOS (MSV), MRAS (RRAS3), MSH type genes, MYB (AMV), MYC, MYCL1 (LMYC), MYCN, NCOA4 (ELE1, ARA70, PTC3), NF1 type genes, NMYC, NRAS, NTRK1 (TRK, TRKA), NUP214 (CAN, D9S46E), OVC, TP53 (P53), PALB2, PAX3 (HUP2), STAT1, PDGFB (SIS), PIM genes, PML (MYL), PMS (PMSL) genes, PPM1D (WIP1), PTEN (MMAC1), PVT1, RAF1 (CRAF), RB1 (RB), RET, RRAS2 (TC21), ROS1 (ROS, MCF3), SMAD type genes, SMARCB1 (SNF5, INI1), SMURF1, SRC (AVS), STAT1, STAT3, STAT5, TDGF1 (CRGF), TGFBR2, THRA (ERBA, EAR7 etc.), TFG (TRKT3), TIF1 (TRIM24, TIF1A), TNC (TN, HXB), TRK, TUSC3, USP6 (TRE2), WNT1 (INT1), WT1, CCDC26, CDKN2BAS, RTEL1, TERT, ERCC1, ERCC2, ERCC5, BRCA2, IDH1/2, NF1, NF2, TSC1, TSC2, PTEN, CASP-9, CAMKK2, P2RX7, MSH6, PDTM25, KDR, VTI1A, ETFA, TMEM127, GSTT1, CHAF1A, RCC1, XRCC1, EME1, ATM, GLTSCR1, XRCC4, GLM2, PTEN, CDKN2A, CDKN2B, p14/ARF, XRCC3, MGMT, XRCC4, MMR, IDH1, ERBB2, CDKN2A, CCDC26, SUFU, NPAS2, CCDKN2A, PTCH2, CTNNB1, P21, RIC8A, CASP8, XRCC1, WRN, BRIP1, SMARCE1, MN1, PDGFB, VHL.

In some embodiments, diseases are caused by the interaction of NAMACS and/or NAMACS-ANA and/or TEZR of intracellular bacteria with host's cell cytosol.

In some embodiments, products are done for the regulation of the interactions of microorganisms in mixed microbial communities, microbial antagonism, including biofilms, including obtaining stable mixtures of microorganisms.

In some embodiments products are done for changing the properties of the cell in order to prevent complications during air/space flights, staying at other planets, therapies and medical intervention, of transplantation (engraftment, rejection, transplant against the host), cancer therapy (chemo-radio-immunotherapy, cytokine release syndrome and other CAR-T therapy side effects).

In some embodiments products are done for changing the properties of the cell modification their activity of immune cells and/or cells targeted by the components of immune system are used to regulate immune response.

In some embodiments products are done for changing the properties of the cell on fecal microbiome transplantation and non-fecal microbiome transplantation (comprised of at least one microorganism species selected from the group consisting of Actinomycetales, Bifidobacteriales, Bacteroidales, Flavobacteriales, Bacillales, Lactobacillies, Firmicutes, Proteobacteria Spirochaetes, Bacteroidetes, Clostridiales, Erysipelotrichales, Selenomonadales, Fusobacteriales, Neisseriales, Campylobacterales, Pasteurellales) aimed to increase the efficacy of such a microbiome transplant for the therapy of human diseases with a non-limiting examples of IBD, Crohn's disease, ulcerative colitis, weight, Chronic Clostridium difficile Infection, colitis, Chronic constipation, Chronic Fatigue Syndrome (CFS), Collagenous Colitis, Colonic Polyps, Constipation Predominant FBD, Crohn's Disease, Functional Bowel Disease, Irritable bowel syndrome, constipation-predominant, IBS diarrhea/constipation alternating, IBS diarrhea-predominant, IBS pain-predominant, Indeterminate Colitis, Microscopic Colitis, Mucous Colitis, Non-ulcer Dyspepsia, Norwalk Viral Gastroenteritis, Pain Predominant FBD, Primary Clostridium difficile Infection, Primary Sclerosing Cholangitis, Pseudomembranous Colitis, Small Bowel Bacterial Overgrowth, NASH, fibrosis, Ulcerative Colitis, and Upper Abdominal FBD, Autoimmune disorders, neurodegenerative disorders with a non-limiting examples of Alzheimer's disease, Parkinson's disease, amyotrophic lateral sclerosis, Multiple Sclerosis, autism, cancers.

In some embodiments products are done in combination with antibiotics to regulate the formation of the spores of spore-forming bacteria.

In some embodiments the treatment and prevention of human diseases, by products usage for managing activity within representatives of microbiota including skin, gut, brain, lung, vaginal, tumor microbiotas.

In some embodiments products are done for changing recipients' or/and donors' tissues for the improved efficacy of tissue and organs transplantation.

In some embodiments products are done for changing the properties of the recipient cells to increase the efficacy of CRISPR, TALEN, ZFN and other gene editing technologies.

In some embodiments products are done for the prevention of NAMACS and/or NAMACS-ANA interaction with proteins.

In some embodiments products are used to produce or modulate: ion channels, brain stimulation, cell signaling within nervous system, e.g. neurons, microglia, modification of responses to cortical stimulation, cell signaling between nervous cells and microglia with a non-limiting example of synaptic transmission, synaptic connectivity between neurons, neuronal excitability and synaptic plasticity, brain ageing, age-related deficits in learning and memory, cognitive decline, brain development, neurotoxicity, excitotoxicity, neurodegeneration, neourodevelopment, sleep disorders, epilepsy.

In some embodiments increase or decrease of DNase and/or RNase activity in human tissues, extracellular space, biofluids (with a non-limiting examples of nervous tissue, brain, cerebrospinal fluid) is used to prevent and treat human diseases.

In some embodiments products can be used to modulate the work of Ca, Na, K, channels.

In some embodiments products are used to modulate electrical properties, polarization, depolarization and extrapolarization of cell's membranes potential.

In some embodiments the decrease of RNase activity in human tissues, extracellular space, biofluids are used to modulate electrical properties and depolarization potential of the cells, polarization, depolarization and extrapolarization of membranes potential with a non-limiting examples of neurons.

In some embodiments products are used for managing activity within axons and/or dendrites and/or synapses.

In some embodiments products are done for managing process of viral and/or capsid surface of various delivery vehicles, including, without limitation, viral vectors (e.g., adeno-associated virus vectors, adenovirus vectors, retrovirus vectors [e.g., lentivirus vectors]) is used to increase the specificity of gene therapy.

In some embodiments products are used for regulation of miRNA, protein expression.

In some embodiments products are done for eukaryotic and prokaryotic cells to alter evolution process.

In some embodiments products are done for control activity within eukaryotic and prokaryotic cells to modulate increased intestinal permeability.

In some embodiments products are done for managing of normal lysosomal function, autophagy, control of protein export from neurons, anti-amyloid therapies (including active immunotherapy), drugs aimed targeting protein aggregation and other methods aimed prevents accumulation of misfolded proteins along or together with drugs having synergistic effects on these processes.

In some embodiments products are done within eukaryotic or prokaryotic cells to restore neuron injury and regeneration of neurons and neurological damage.

In some embodiments alteration of NAMACS and/or NAMACS-ANA and/or TEZRs including the use of artificial ones and/or are done for formation of system for signal transferring and cellular cooperation and as an analogue of nervous system bringing signals between cells, cell groups, tissues, organs and their qualitative or quantitative change of can be used for the modification of such a signaling.

In some embodiments analysis of NAMACS and/or NAMACS-ANA and/or TEZRs are used to assess the effectiveness drugs in clinical trials.

In some embodiments products are done for managing of stem cells differentiation.

In some embodiments products are done for managing of embryo cells affect the embryogenesis.

In some embodiments products can be used to modulate the efficacy of transmitters formation, release and effects of glutamate, aspartate, D-serine, y-aminobutyric acid (GABA), glycine, nitric oxide, carbon monoxide, hydrogensulfide, dopamine, norepinephrine (noradrenaline), epinephrine (adrenaline), histamine, serotonin, phenethylamine, N-methylphenethylamine, tyramine, 3-iodothyronamine, octopamine, tryptamine, oxytocin, somatostatin, substance P, cocaine and amphetamine regulated transcript, opioid peptides, adenosine triphosphate (ATP), adenosine, dopamine, acetylcholine, anandamide, etc.

In some embodiments products can be used to regulate work of nocioreceptors and/or opioid receptors and/or mechanoreceptors and/or magnetoreceptors and/or chemoreceptors is associated with.

In some embodiments products manage the release or effects of neutrophil extracellular traps.

In some embodiments products manage surgical outcomes, and/or can be used in vivo or ex vivo for pretransplant organ reconditioning.

In some embodiments products are used to treat drug overdose including opioids, drug abuse, prophylactic and treatment of morphine and other drugs overdose, respiratory depression, neuropathic pain, gastrointestinal disfunction, addictions and substance use disorders.

In some embodiments products are used to regulate interferon-dependent cell protection.

In some embodiments products are used to regulate hormones levels, cells sensitivity to hormones with a non-limiting examples of insulin.

In some embodiments products are done for increase and/or decrease and/or modification cells activity with the use of skin products (cream, tonic, etc.).

In some embodiments products are done for mammalian cells affect longevity assurance mechanisms resulting in delay of DNA damage-driven aging.

In some embodiments products affect longevity by alteration of mechanisms resulting in delay of DNA damage-driven aging activity is used to regulate DNA repair, DNA recombination, regulation of intragenomic rearrangements, the behavior of prophages, plasmids, transposons and other mobile genetic elements, regulation of protein synthesis in cells.

In some embodiments products usage can lead to the dysfunction of receptors with a non-limiting examples of tyrosin-kinase-based receptors such as EGFR, Tumor necrosis factor related apoptosis-inducing ligand, TLRs, Serotonin receptors, CTLA-4, PD-1, and PD-L1, PD-L2, B7 family, VISTA, Tim-3 and LAG-3, TCR, MHC, Gal-9, MHCII, HHLA2, LSECtin, CD80/86, CD4, CD3, CD28, TIL, estrogen receptor, progesterone receptor, human epidermal growth factor receptor, VEGF, VEGFR, RYK, GDNF, RET, ERBB, INSR, IGF-1R,IRR, PDGFR, CSF-1R, KIT/SCFR, FLK2/FLT3, FGFR, CCK4, TRKS, TRKB, TRKC, MEN, RON, EPHA, AXL, MER, TYRO, TIE, TEK, DDR, ROS, LTK, ALK, ROR, MUSK, AATYK, RTK, FLT3, JAK3, FAK, BCR, TCR, INSR group, FGFR group, EGFR group, EPH group, ROR group; and that affect signaling pathway with a non-limiting examples of those associated with WNT, SRC, PI3K, PTEN, AKT, mTOR, PARP, CHK1/2, WEE, insulin, opioid, and can be used alone or in combination with other drugs targetnig such a receptors with a non-limiting examples of monoclonal antibodies (mAbs) that target the extracellular domain and/or receptor catalytic domains, and/or can be used to overcome drug-resistance mutations of such a receptors, with a non-limiting example to affect aberrant protein phosphorylation.

In some embodiments alterations of cellular memory by products is inherited to the next generation of cells.

### In Some Embodiments

In one embodiment, the addition of cells in “Cut”, “Zero”, “Y” states to the organism can cause cascade alterations of other cells, leading to a health beneficial effects including rejuvenation within 24h post their administration, from 1 day to 1 week, in a month, in a 6 month, in a year, during the time to 5 years, during the time to 10 years, during the time to 20 years, during the time to 50, during the time to 80 years, during the time to 120 years.

In one embodiment, NAMACS and/or NAMACS-ANA and/or TezRs of one cell and/or tissue and/or organism interact with the TezRs of another cell and/or tissue and/or organism.

In one embodiment, NAMACS and/or NAMACS-ANA and/or TezRs regulate electrostatic interactions, hydrophobic interactions of cellular components.

In one embodiment, NAMACS and/or NAMACS-ANA and/or TEZRs are used to regulate biological rhythms including circadian rhythms.

In some embodiments NAMACS and/or NAMACS-ANA and/or TEZRs can make cells immortal or increase maximum number of cell divisions.

In some embodiments products are used to generate naïve state of the cells more sensitive or resistant for physical, chemical, mechanical, biological factors.

In some embodiments products can be used to increase production of cells or/and their metabolites used in biotechnological applications.

In some embodiments including to control the synthesis and/or synthesis and/or secretion of DNA and/or RNA and/or proteins.

In some embodiments NAMACS and/or NAMACS-ANA and/or TEZRs are used to regulate work of cell receptors including their interactions with ligands.

In some embodiments products are used to increase production of energy by cells.

In some embodiments products are used to control regeneration.

In some embodiments products are used control differentiation of cells for the prevention and treatment of diseases and creation of organisms with new characteristics.

In some embodiments products are used to obtain altered immune system cells and/or stem cells and/or mammalian and/or plant cells suitable for embriogenesis and to prevent the development of congenital defects, and can be used for artificial insemination.

In some embodiments products treatment of seeds, plants, are used for plant breeding and/or selection processes and/or regulation of plant productivity.

In some embodiments eukaryotes and prokaryotes are treated with products to modulate and control food and beverages fermentation.

In some embodiments products are used for increase productivity of eukaryotic and prokaryotic cells, master cell line containing the gene that makes the desired proteins in biotechnology (e.g. associated with recombinant DNA and RNA; Amino acids; Biopharmaceuticals; Cytokines; Fusion proteins; Growth factors; Clotting and coagulation factors; TNF inhibitors; Interferons, Antibodies; Recombinant Antibodies; Recombinant proteins; AAVs, viruses, Antibodies; Vaccines, Vectors, Receptors, Hormones).

In some embodiments products are used to change activity of plants and/or plant seeds before and/or after planting of agricultural plants.

In some embodiments products can be used for the production of bioenergy.

In some embodiments products are used for managing the energetic, glycemic, oxidation state of the cells, tissues, organs.

In some embodiments products can be used to increase transport of external molecules to the cell or secretion and excretion from the cells.

In some embodiments products are used to can be used to modulate bacterial, fungal, mammalian, or plant metabolism.

In some embodiments products are used to can be used to modulate energy state of the cells (e.g. ATP content in cells) or prevention of recurrent formation ATP content in cells.

In some embodiments products can modulate anaerobic survival metabolisms in aerobes (both prokaryotes and eucaryotes) with a non-limiting example of regulation of microbial colonization of the gut, site of anaerobic infections, outer space, places with a poorly vascularization.

In some embodiments products can modulate anaerobic cellular respiration and/or fermentation generate ATP under aerobic and anaerobic environments, and/or effects on NADH and FADH2 metabolism and/or ion channels and ionic passage.

In some embodiments products can be used to modulate somatic mosaicism.

In some embodiments products are used for the development of artificial organs and organisms.

In some embodiments products are used for the treatment of human diseases, including migraine, meteo-dependence, headaches.

In some embodiments products are used for the treatment of human diseases, including migraine, weather-dependence, headaches are replaced by other microorganisms without TEZRs.

In some embodiments products can be used to target pathways include KRAS/ERK/MEK, PI3K/AKT/mTOR, JAK-STAT, and FAK/SRC, WNT signaling, heat shock regulation, glycogen synthase kinase 3 (GSK-3), and transforming growth factor beta (TGFβ).

### FIGURES

FIG. 1 shows the effect of treated cells and their supernatant on solid tumors. (A) and (B) Reduction in tumor volume following treatment with SL4-T treated cells.

FIG. 2 shows a reduction of a squamous cell carcinoma located in the oral cavity floor over time following treatment.

FIG. 3 shows a reduction of an adenocarcinoma located in the right lung over time following treatment.

FIG. 4 shows a reduction of a Krukenberg tumor over time following treatment.

FIG. 5 shows a reduction of a Krukenberg tumor over time following treatment.

FIG. 6 shows a reduction in bilateral adrenal neuroblastoma neoplasm of retroperitoneum over time following treatment.

FIG. 7 shows the in vitro viricidal activity of SL4-T.

FIG. 8 shows the effect of cell treatment on Qp with CHO-T and CHO-Ta cells.

FIG. 9 shows the formation of Guttation Droplets with antibiotic on the Surface of Fungal Colonies.

FIG. 10 shows the effect of cell treatment on the production of proteins over time.

FIG. 11 shows the use of SL4 treated cells for the treatment of inflammatory diseases.

FIG. 12 shows the effect of treated cells on PASI score and representative skin images of patients before and after treatment with treated cells from group C and representative skin images of patients before and after treatment with treated cells from group C.

FIG. 13 shows the effect of treated cells on PASI score and representative skin images of patients before and after treatment with treated cells from group C and representative skin images of patients before and after treatment with treated cells from group C

FIG. 14 shows the effect of using treated cells to treat diabetes over time.

FIG. 15 shows a reduction in the size of osteochondral defect areas following treatment for one month.

FIG. 16 shows the effect of treatment RBC on Rh antigen for different blood types.

FIG. 17 shows a Bray-Curtis similarity between pairs of inoculum and donor samples.

FIG. 18 shows immune cell signaling.

FIG. 19 shows cellular uptake.

FIG. 20 shows cell migration.

FIG. 21 shows the effect of treated cells on the generation of evolutionary old ancestor organisms.

### DETAILED DESCRIPTION

The Universal Receptive System is present in both prokaryotic and eukaryotic organisms. DNA and RNA located outside the cell membrane form Teazaled receptors (TezRs), which exhibit receptor and regulatory functions. TezRs work extracellularly, alongside integrases and trascriptases to constitute the Universal Receptive System This system has both receptive and regulatory functions. The Universal Receptive System operates by, serving as a recipient of external stimuli. These can include, but are not limited to temperature fluctuations, light changes, and geomagnetic fields. TezRs also integrate and modulate signals from pathways associated with conventional protein-based receptors exposed on the surface of cells.

Targeted manipulation of the Universal Receptive System of prokaryotic and eukaryotic cells can regulate key cellular features that lead to the emergence of novel cell states and properties, without directly affecting the genome of the cell. Manipulation of the Universal Receptive System can effect various chemical, physical, and biological factors, including, but not limited to the control of cell memory, the elimination of cell memory (forgetting) or changes in gene expression patterns (e.g., illustrated by treatment-induced, differential expression of genes involved in essential cell processes, including metabolism, proliferation, apoptosis, and protein expression. The Universal Receptive System also controls some of the fundamental properties of immune cells, including, but not limited to, the production of diverse bioactive molecules, including antimicrobial and antitumor compounds by leucocytes. The targeting of the Universal Receptive System across diverse cellular systems can thus elucidate significant modulatory effects on cellular activity and can be used as a tool to engineer novel cellular states.

DNA- and RNA-based TezRs can act as receptors and/or regulators of extracellular signals and highlights the role of DNA and RNA in cellular functions not related to encoding genetic information and protein synthesis. This extracellular DNA and RNA can interact with proteins, a process known as the “Pliers function.” The Pliers function induces altered protein folding and the generation of novel protein isoforms. These alterations lead to changes in how the protein functions, including some that are associated with pathogenic properties.

The deactivation of recently discovered TezRs has several effects on the cell. These include the emergence of leucocytes that have been called “Leukocyte-Tells” which result following treatment with a DNase, an RNase, an antibody that binds to a DNA or an antibody that binds to an RNA. These Leukocyte-Tells have an upregulated antimicrobial and anticancer activities that is greater than untreated leukocytes. This antimicrobial and anticancer activity is mediated by “Anticancer and Antimicrobial Bioactive Compounds” (AABC), which can be divided into several categories based on their chemical structure. These categories, include, but are not limited to peptides, proteins, and metabolites.

Reverse transcriptase inhibitors include Nevirapine, Penciclovir, Tenofovir disoproxil, Zidovudine, Foscarnet, Efavirenz, Stavudine, Delavirdine, Lamivudine, Adefovir dipivoxil Etravirine Abacavir, Integrase/recombinase inhibitors that include (raltegravir, Dolutegravir, Bictegravir, Cabotegravir, 2,8-dichloro-5-(4-nitrophenyl)-5,9-dihydro-4H-pyrimido[5′,4′:5,6] pyrano[2,3-d]pyrimidine-4,6 (1H)-dione (VTL).

In an embodiment, a treated cell is a cell that has been treated with one or more of a DNase, an RNase, an antibody that binds to a DNA and an antibody that binds to an RNA. In an embodiment, a treated cell is first treated with a DNase and then an RNase or an RNase and then a DNase. In a further embodiment, a treated cell is first treated with a DNase and then an antibody that binds to an RNase or an antibody that binds to an RNase and then a DNase. In another embodiment, a treated cell is first treated with an RNase and then an antibody that binds to a DNA or an antibody that binds to a DNA and then an RNase. For any of the above treatments, a treated cell can be treated with one or more of a DNase, an RNase, an antibody that binds to a DNA or an antibody that binds to an RNA two or more times and the treatment can be any combination of the one or more of a DNase, an RNase, an antibody that binds to a DNA or an antibody that binds to an RNA.

Treatment of the cells results in an improvement of one or more characteristics of the treated cells. For instance, for a treated cell used to manufacture a bioproduct, like a protein (including a monoclonal antibody, a bivalent or trivalent protein, insulin or other protein used to supplement a protein deficiency), the treated cell is capable of producing greater quantities of the protein. In another example, the treated cell is a B-cell, wherein, following treatment the B-cell either is able to produce a greater quantity of antibodies, or the B-cell is made naïve again and ready to respond to a new antigenic challenge. For a treated cell used to make a plant that constitutes a source of food (including, a grain, a wheat, a fruit, a tomato, a peach, an orange, an apricot, a grape, a hybrid fruit, an apple, a nut, a pecan, a walnut, an almond, a pistachio, a peanut, a coffee bean, a tea, a soybean, a corn, a pea, a carrot, broccoli, squash, cauliflower, a mushroom, a bean, a legume, a barley, a sorgum, a legume or other plant food source), the plant produces grows larger, faster, produces more edible or usable product and/or provides the food with another favorable characteristic.

In another embodiment, a treated cell is a bacteria, a fungi or other cellular organism that is capable of producing a protein, including an antibiotic. In this embodiment, the treated cell is capable of producing greater quantities of an antibiotic or produce an antibiotic that is more efficacious in killing bacteria.

In other aspects of this embodiment, a cell treated with a DNase, an RNase, an antibody that binds to a DNA or an antibody that binds to an RNA is/are treated for 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 24 hours, 48 hours, 72 hours, or more hours. In other aspects of this embodiment, a cell treated with a DNase, an RNase, an antibody that binds to a DNA or an antibody that binds to an RNA is treated for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 21 minutes, at least 22 minutes, at least 23 minutes, at least 24 minutes, at least 25 minutes, at least 26 minutes, at least 27 minutes, at least 28 minutes, at least 29 minutes, at least 30 minutes, 3 at least 5 minutes, at least 40 minutes, at least 45 minutes, at least 50 minutes, at least 55 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, 24 hours, 48 hours, 72 hours, or more hours. In other aspects of this embodiment, a cell treated with a DNase, an RNase, an antibody that binds to a DNA or an antibody that binds to an RNA is/are treated for no more than 1 minute, no more than 2 minutes, no more than 3 minutes, no more than 4 minutes, no more than 5 minutes, no more than 6 minutes, no more than 7 minutes, no more than 8 minutes, no more than 9 minutes, no more than 10 minutes, no more than 11 minutes, no more than 12 minutes, no more than 13 minutes, no more than 14 minutes, no more than 15 minutes, no more than 16 minutes, no more than 17 minutes, no more than 18 minutes, no more than 19 minutes, no more than 20 minutes, no more than 21 minutes, no more than 22 minutes, no more than 23 minutes, no more than 24 minutes, no more than 25 minutes, no more than 26 minutes, no more than 27 minutes, no more than 28 minutes, no more than 29 minutes, no more than 30 minutes, 3 no more than 5 minutes, no more than 40 minutes, no more than 45 minutes, no more than 50 minutes, no more than 55 minutes, no more than 1 hour, no more than 2 hours, no more than 3 hours, no more than 4 hours, no more than 5 hours, no more than 6 hours, no more than 7 hours, no more than 8 hours, no more than 9 hours, no more than 10 hours, no more than 11 hours, no more than 12 hours, 24 hours, 48 hours, 72 hours, or more hours. In other aspects of this embodiment, a cell is/are treated with a DNase, an RNase, an antibody that binds to a DNA or an antibody that binds to an RNA for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, 3 about 5 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, 24 hours, 48 hours, 72 hours, or more hours.

In other aspects of this embodiment, a cell treated with a DNase, an RNase, an antibody that binds to a DNA or an antibody that binds to an RNA is/are treated 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 35 times, 40 times, 45 times, 50 times, 55 times, 100 times or more times with the nuclease(s) and/or antibody (ies).

In other aspects of this embodiment, the time between multiple rounds of cell treatment with a DNase, an RNase, an antibody that binds to a DNA or an antibody that binds to an RNA is 1 minute, 5 minutes, 10 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 48 hours, 72 hours, or more hours with the nuclease(s) and/or antibody (ies).

In aspects of this embodiment, a cell treated with a DNase, an RNase, an antibody that binds to a DNA or a cell that binds to an RNA, is/are treated with no more than 1 pg, no more than 100 pg, no more than 5 ng, no more than 50 ng, no more than 500 ng, no more than 1 mcg, no more than 10 mcg, no more than 40 mcg, no more than 100 ng, no more than 250 mcg, no more than 500 mcg, no more than 1 mg, no more than 5 mg, no more than 10 mg, no more than 100 mg of the nuclease or antibody per ml. In other aspects of this embodiment, a cell treated with a DNase, an RNase, an antibody that binds to a DNA or a cell that binds to an RNA, is treated with at least 5 ng, at least 50 ng, at least 500 ng, at least 1 mcg, at least 10 mcg, at least 40 mcg, at least 100 ng, at least 250 mcg, at least 500 mcg, at least 1 mg, at least 5 mg, at least 10 mg, at least 100 mg of the nuclease or antibody per ml. In yet other aspects of this embodiment, a cell treated with a DNase, an RNase, an antibody that binds to a DNA or a cell that binds to an RNA, is treated with about 5 ng, about 50 ng, about 500 ng, about 1 mcg, about 10 mcg, about 40 mcg, about 100 ng, about 250 mcg, about 500 mcg, about 1 mg, about 5 mg, about 10 mg, about 100 mg per ml of the nuclease or antibody. In still other aspects of this embodiment, a cell treated with a DNase, an RNase, an antibody that binds to a DNA or a cell that binds to an RNA, is treated with about 10 ng to about 1 mcg, about 10 ng to about 10 mcg, about 10 ng to about 50 mcg, about 10 ng to about 100 mg, about 10 ng to about 200 mg, about 50 ng to about 250 mcg, about 50 ng to about 500 mcg, about 50 ng to about 1 mg per ml of the nuclease or antibody.

A treated cell that is to be administered to an individual is administered in a solvent, an emulsion or other diluent in an amount sufficient to maintain the stability of the treated cell prior to and as necessary, following administration. In other aspects of this embodiment, a treated cell that is to be administered to an individual is administered in a solvent, an emulsion or other diluent in an amount sufficient to maintain the stability of the treated cell prior to and as necessary, following administration of an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a pharmaceutical composition disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

In an aspect of this embodiment, a cell treated with a DNase, an RNase, an antibody that binds to a DNA or a cell that binds to an RNA, is/are treated with at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In other aspects of this embodiment, the concentration of a pharmaceutical composition disclosed herein in the solution may be, e.g., at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/ml of a nuclease or an antibody. In other aspects of this embodiment, a cell treated with a DNase, an RNase, an antibody that binds to a DNA or a cell that binds to an RNA, is treated with is about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/ml of an antibody or nuclease.

Aspects of the present specification disclose, in part, treating an individual suffering from a clinical syndrome or disease. As used herein, the term “treating,” to the extent it does not refer to treating a cell with a nuclease or an antibody refers to reducing or eliminating in an individual a clinical syndrome or disease; or delaying or preventing in an individual the onset of a clinical syndrome or disease. For example, the term “treating” can mean reducing a symptom of a condition characterized by a syndrome or disease, including a reduction or elimination of pain or expediting the growth of new tissue, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with the disclosed syndromes and diseases are well known and can be determined by a person of ordinary skill in the art by taking into account factors, including, without limitation, the location of the syndrome or disease in the body, including pain, the location of the pain and the genesis of the pain. Those of skill in the art will know the appropriate symptoms or indicators associated with a specific type of syndrome or diseases, including pain and will know how to determine if an individual is a candidate for treatment as disclosed herein.

In aspects of this embodiment, a therapeutically effective amount of a treated cell reduces a symptom associated with a disease (including a cancer, an autoimmune disease, a hormonal disease, an infectious disease (including bacterial, fungal, viral and or parasitic), autoimmune disorders, neurodegenerative diseases, a rare disease (including hemophilia) a gastric disease, liver disease, kidney disease, a cardiovascular disease, a nerve disorder or a urinary disease) by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a treated cell reduces a symptom associated with a disease (including a cancer, an autoimmune disease, a hormonal disease, an infectious disease (including bacterial, viral and or parasitic), diabetes, neurodegenerative diseases, a rare disease (including hemophilia) a gastric disease, liver disease, kidney disease, a cardiovascular disease, a nerve disorder or a urinary disease) by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a treated cell reduces a symptom associated with a disease (including a cancer, an autoimmune disease, a hormonal disease, an infectious disease (including bacterial, viral and or parasitic), diabetes, neurodegenerative diseases, a rare disease (including hemophilia) a gastric disease, liver disease, kidney disease, a cardiovascular disease, a nerve disorder or a urinary disease) by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of this embodiment, a treated cell reduces the number of a bacteria, a virus, a biofilm, a fungus or a parasite by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a treated cell reduces the number of a bacteria, a virus, a biofilm, a fungus or a parasite by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a treated cell reduces the number of a bacteria, a virus, a biofilm, a fungus or a parasite by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of this embodiment, a treated cell that produces a bioproduct (an antibiotic, an anti-viral, an anti-fungal, anticancer, or anti-biofilm bioproduct) reduces the number of a bacteria, a virus, a biofilm, a fungus, cancer cell or a parasite by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a treated cell that produces a bioproduct (an antibiotic, an anti-viral, an anti-fungal anticancer, or anti-biofilm bioproduct) reduces the number of a bacteria, a virus, a biofilm, a fungus, cancer cell or a parasite by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a treated cell that produces a bioproduct (an antibiotic, an anti-viral, an anti-fungal anticancer, or anti-biofilm bioproduct) reduces the number of a bacteria, a virus, a biofilm, a fungus, cancer cell or a parasite by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of this embodiment, a plant cell for a plant used to generate food that is treated with an RNA, a DNA, an RNA binding antibody and/or a DNA binding antibody is capable of producing a plant that produces at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100% more plant bioproduct (seed, fruit, grain, bean, legume and/or root) than a plant cell that is not treated with an RNA, a DNA, an RNA binding antibody and/or a DNA binding antibody. In other aspects of this embodiment, a plant cell for a plant used to generate food that is treated with an RNA, a DNA, an RNA binding antibody and/or a DNA binding antibody is capable of producing a plant that produces at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% % more plant bioproduct (seed, fruit, grain, bean, legume and/or root) than a plant cell that is not treated with an RNA, a DNA, an RNA binding antibody and/or a DNA binding antibody. In yet other aspects of this embodiment, a plant cell for a plant used to generate food that is treated with an RNA, a DNA, an

RNA binding antibody and/or a DNA binding antibody is capable of producing a plant that produces about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50% % more plant bioproduct (seed, fruit, grain, bean, legume and/or root) than a plant cell that is not treated with an RNA, a DNA, an RNA binding antibody and/or a DNA binding antibody.

In aspects of this embodiment, following treatment of a cell with an RNase, a DNase, an antibody that binds to an RNA or an antibody that binds to a DNA, the treated cell produces at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100% more bioproduct (including a protein or a peptide) than the same cell if not treated with an RNase, a DNase, an antibody that binds to an RNA or an antibody that binds to a DNA. In other aspects of this embodiment, following treatment of a cell with an RNase, a DNase, an antibody that binds to an RNA or an antibody that binds to a DNA, the treated cell produces at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% more bioproduct (including a protein or a peptide) than the same cell if not treated with an RNase, a DNase, an antibody that binds to an RNA or an antibody that binds to a DNA. In yet other aspects of this embodiment, following treatment of a cell with an RNase, a DNase, an antibody that binds to an RNA or an antibody that binds to a DNA, the treated cell produces about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50% more bioproduct (including a protein or a peptide) than the same cell if not treated with an RNase, a DNase, an antibody that binds to an RNA or an antibody that binds to a DNA.

In yet other aspects of this embodiment, an RNase, a DNase, an antibody that binds to an RNA and/or an antibody that binds to a DNA is administered to a patient in an amount of about 0.001 mg/kg/day to about 100 mg/kg/day. In aspects of this embodiment, an RNase, a DNase, an antibody that binds to an RNA and/or an antibody that binds to a DNA is administered to a patient in an amount of at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day. In other aspects of this embodiment, an RNase, a DNase, an antibody that binds to an RNA and/or an antibody that binds to a DNA is administered to a patient in an amount of about 0.001 mg/kg/day to about 10 mg/kg/day, about 0.001 mg/kg/day to about 15 mg/kg/day, about 0.001 mg/kg/day to about 20 will mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day. In yet other aspects of this embodiment, an RNase, a DNase, an antibody that binds to an RNA and/or an antibody that binds to a DNA is administered to a patient in an amount of about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day. In still other aspects of this embodiment, an effective amount of a pharmaceutical composition disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day.

In one embodiment, a treated cell (including a B cell, a T cell, a CAR-T cell, a dendritic cell, a neutrophil, a natural killer cell, a leukocyte and/or a macrophage) disclosed herein is capable of reducing the number of cancer cells or tumor size in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a treated cell (including a B cell, a T cell, a CAR-T cell, a dendritic cell, a neutrophil, a natural killer cell, a leukocyte and/or a macrophage) is/are capable of reducing the number of cancer cells or tumor size in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

Dosing of a treated cell to an individual can be through a single dosage or cumulative (serial dosing), and can be readily determined by one skilled in the art. For instance, treatment of a cancer may comprise a one-time administration of an effective number of treated cells (the dose) as disclosed herein. Alternatively, treatment of a cancer may comprise multiple administrations of an effective number of cells of a treated cells (the dose) carried out over a range of time periods, such as, e.g., once daily, twice daily, trice daily, once every few days, or once weekly. The timing of administration can vary from individual to individual, depending upon such factors as the severity of an individual's symptoms or a requirement for a specific amount of bioproduct by a treated cell. For example, an effective dose of a treated cell disclosed herein can be administered to an individual once daily for an indefinite period of time, or until the individual no longer requires therapy. A person of ordinary skill in the art will recognize that the condition of the individual can be monitored throughout the course of treatment and that the effective amount of a treated cell disclosed herein that is administered can be adjusted accordingly.

In a further embodiment, a treated cell (including a B cell, a T cell, a CAR-T cell, a dendritic cell, a neutrophil, a natural killer cell, a leukocyte and/or a macrophage) has/have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a treated cell (including a B cell, a T cell, a CAR-T cell, a dendritic cell, a neutrophil, a natural killer cell, a leukocyte and/or a macrophage) is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a treated cell (including a B cell, a T cell, a CAR-T cell, a dendritic cell, a neutrophil, a natural killer cell, a leukocyte and/or a macrophage) disclosed herein reduces or maintains a cancer cell population and/or tumor cell size in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a treated cell (including a B cell, a T cell, a CAR-T cell, a dendritic cell, a neutrophil, a natural killer cell, a leukocyte and/or a macrophage) disclosed herein reduces or maintains a cancer cell population and/or tumor cell size in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a treated cell (including a B cell, a T cell, a CAR-T cell, a dendritic cell, a neutrophil, a natural killer cell, a leukocyte and/or a macrophage) disclosed herein reduces or maintains a cancer cell population and/or tumor cell size in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A treated cell is administered to an individual. An individual is typically a human being, but can be an animal, including, but not limited to, dogs, cats, birds, cattle, horses, sheep, goats, reptiles and other animals, whether domesticated or not. Typically, any individual who is a candidate for treatment is a candidate with some form of a disease or syndrome, including a cancer, an autoimmune disease, a hormonal disease, an infectious disease (including bacterial, viral and or parasitic), diabetes, a rare disease (including hemophilia) a gastric disease, liver disease, kidney disease, a cardiovascular disease, a nerve disorder or a urinary disease. If the disease or syndrome is a cancer, the cancer is either a benign or malignant cancer. The cancer is a tumor, solid or otherwise, a cancer cell not located in a tumor or some other form of cancer. Among the most common types of cancer include, but are not limited to, bladder cancer, breast cancer, colon and rectal cancer, endometrial cancer, kidney cancer, renal cancer, leukemia, lung cancer, melanoma, non-Hodgkins lymphoma, pancreatic cancer, prostate cancer, stomach cancer and thyroid cancer. Pre-operative evaluation typically includes routine history and physical examination in addition to thorough informed consent disclosing all relevant risks and benefits of the procedure.

Immune cells means any cells involved in the immune response. Immune cells include, but are not limited to, B-cells, T-cells, dendritic cells, macrophages, natural killer cells, neutrophils, monocytes. leucocytes, eosinophils, monocytes, basophils, plasma cells, CD34+ cells, cells of microglia and mast cells.

Treated Cells Means any Cells Treated with One or More of a DNase, an RNase, an Anti-DNA Antibody or an Anti-RNA Antibody, Wherein the Cells are Treated One or More Times. Treated Cells can be Treated In Vitro

In an embodiment, immune cells are treated with a DNase or an RNase in vitro, after which the treated immune cells are administered to an individual suffering from a cancer. The cancer can be a cancer that comprises a solid tumor or circulating cancer cells. In another embodiment, the immune cells are treated with one or more of a DNase or RNase one or more times in vitro prior to administration to an individual suffering from a cancer. The treated cells are administered to an individual to prevent and treat a metastasis.

In an embodiment, immune cells are treated with an anti-DNA antibody or an anti-RNA antibody in vitro, after which the treated immune cells are administered to an individual suffering from a cancer. The cancer can be a cancer that comprises a solid tumor or circulating cancer cells. In another embodiment, the immune cells are treated with an anti-DNA antibody or an anti-RNA antibody one or more times in vitro prior to administration to an individual suffering from a cancer. The treated cells are administered to an individual to treat a metastasis.

The cells treated with one or more of a DNase, an RNase, an anti-RNA antibody and/or anti-DNA antibody in vitro are administered to a patient suffering from one or more of the following cancers: carcinoma, Sarcoma, Leukemia, Myeloma, Lymphoma, Central Nervous System Cancers, Germ Cell Tumors, lung cancer, pancreatic cancer, bladder cancer, stomach cancer, colon cancer, brain cancer, glioblastoma, acute myeloid leukemia, acute lymphoblastic leukemia, bone cancer, breast cancer, Burkitt lymphoma, cervical cancer, chronic lymphocytic leukemia, chronic myelogenous leukemia, chronic myeloproliferative neoplasms, colorectal cancer, cutaneous T-cell lymphoma, endometrial cancer, esophageal cancer, Ewing sarcoma, retinoblastoma, gallbladder cancer, testicular cancer, ovarian germ cell tumors, hairy cell leukemia, head and neck cancer, liver cancer, Hodgkin's lymphoma, kidney cancer, Kaposi sarcoma, melanoma, mesothelioma, metastatic cancer, mouth cancer, neuroblastoma, neuroendocrine tumors, non-Hodgkin's lymphoma, non-small cell lung cancer, oral cancer, ovarian cancer, osteosarcoma, parathyroid cancer, penile cancer, pituitary tumor, plasma cell neoplasm, multiple myeloma, primary central nervous system lymphoma, prostate cancer, primary peritoneal cancer, rectal cancer, recurrent cancer, salivary gland cancer, sarcoma, skin cancer, small cell lung cancer, small intestine cancer, soft tissue carcinoma, squamous cell carcinoma, T-cell lymphoma, testicular cancer, throat cancer, thyroid cancer, urethral cancer, uterine cancer, vaginal cancer, vascular tumors and vulvar cancer.

The cells treated with one or more of a DNase, an RNase, an anti-RNA antibody and/or anti-DNA antibody in vitro are administered by autologous and/or allogeneic administration.

The cells treated with one or more of a DNase, an RNase, an anti-RNA antibody and/or anti-DNA antibody in vitro are administered by autologous and/or allogeneic administration, and can originate from different organisms including mammalians or cold-bloodied organisms.

The cells treated with one or more of a DNase, an RNase, an anti-RNA antibody and/or anti-DNA antibody in vitro can be additionally treated with microbial cells or viruses or their components (e.g., LPS,), eukaryotic cells, tumor cells/tissues, mitogens like ConA, or PMA/ionomycin that have been previously treated with one or more of a DNase, an RNase, an anti-RNA antibody and/or anti-DNA antibody.

The cells treated with one or more of a DNase, an RNase, an anti-RNA antibody and/or anti-DNA antibody in vitro are administered to a patient together with, prior to, or after chemotherapy, immunotherapy or surgical therapy of cancer.

Chemotherapeutic agents can include, but are not limited to, those found in Table 1.

Types of immunotherapy can include, but are not limited to, the administration of antibodies that target and bind to specific antigens, including antigens of cancer cells. Immunotherapy can also comprise the administration of one or more of a cancer vaccine, adoptive cell transfer, tumor-infecting viruses, checkpoint inhibitors, cytokines, and adjuvants to a patient suffering from a cancer.

Types of surgical therapy to treat a cancer can include, but are not limited to, cryosurgery, electrosurgery, laser surgery, Moh's surgery, laparascopic surgery, robotic surgery and natural orifice surgery.

The cells treated with one or more of a DNase, an RNase, an anti-RNA antibody and/or anti-DNA antibody in vitro are administered systemically or locally at the site of a tumor to treat the tumor. Other forms of administration of the treated cells include intrapleural, intraabdominal, injection into the wound site that occurs either prior to, during or after the tumor is removed from the patient. By administering the treated cells to a patient suffering from a tumor, the treated cells reduce the size of the tumor or eliminate the tumor from the patient.

In an embodiment, the cells that are treated with one or more of a DNase, an RNase, an anti-RNA antibody and/or anti-DNA antibody in vitro are then administered to a patient in a controlled release formulation. In another embodiment, the controlled release formulation comprise a tablet, a liquid, a powder, a nanoparticle, a controlled release device, subcutaneous autoinjectors. Following administration of the treated cells in a controlled release formulation, the treated cells are released in a controlled release manner.

In an embodiment, cells are treated in vitro with one or more rounds of DNase and RNase or anti-DNA and anti-RNA antibody and the treated cells are used for the treatment or prevention of a bacterial, a fungal, a protozoan and/or a viral infection. In an embodiment, the infection can be a chronic infection, a temporary infection, a repeated infection and/or an acute infection.

In an embodiment, cells are treated in vitro with one or more rounds of DNase and RNase or anti-DNA and anti-RNA antibody and the treated cells are used for the treatment of microbial, including bacterial and viral persisters, a microbial biofilm, including those caused by drug-resistant bacterial strains. A biofilm is a single biofilm or a mixed biofilm.

In a further embodiment, cells are treated in vitro with one or more rounds of DNase and RNase or anti-DNA and anti-RNA antibody and the treated cells are used to treat a bacteria, a virus and/or a parasite that causes an infection. In an embodiment an infection results in an ulcer, sepsis, malaria, a mycobacteriosis, pneumonia, or other disease resulting from a bacterial, viral or parasitic infection.

In another embodiment, the treated cells are selected from a White blood cell, a fibroblast, a platelet, a hematopoietic stem cell, a red blood cell and/or a stem cell. A stem cell can be a haematopoeitc stem cells, a mesenchymal stem cell, a skeletal stem cell, embryonic stem cells, adult stem cells, mesenchymal stem cells, pluripotent stem cells. A white blood cell is selected from a leucocyte, a lymphocyte (including a T-cell, a B-cells, an NK cell), a neutrophil, an eosinophil, a monocyte, a basophil, a macrophage and/or a plasma cells, a CD34+ cell and/or a cell of microglia.

In an embodiment, treated cells are used for autologous and allogeneic administration. In a further embodiment, treated cells are obtained from different organisms. An organism can be a prokaryotic or a eukaryotic organism. An organism can be a mammal, a reptile, a bacteria, a parasite, yeast, a bird, a cold blooded organism.

The therapeutic activity of treated cells can be increased if the treated cells prior to, during or after treatment with a DNase or an RNase or an anti-RNA or anti-DNA antibody were additionally further treated with a microbial cell, a virus, a eukaryotic cell, a tumor cell, a tissue or pieces or derivatives of their components (including cell membranes, organelles, LPS, nucleic acids, proteins, cellular extracts), a mitogen (including ConA, PMA or ionomycin).

In an embodiment, administration of a treated cell is/are by inhalation, intrapleural, intraabdominal, inhalation, intravaginal, intrarectorally, intramuscular, subcutaneous, intraperitoneal, intraocular, orally, intraventricular, local, and/or intraspinal.

In an embodiment, a cell is/are treated in vitro with one or more rounds of a DNase and an RNase and/or an anti-DNA and an anti-RNA antibody, wherein the treated cells are used for the treatment, prevention or reduction of the progression of an idiopathic and/or an autoimmune disease.

An idiopathic and/or autoimmune disease is selected from one of diabetes, an autoimmune disease (including those with gastrointestinal involvement and those with skin involvement), a neurodegenerative disease, multiple sclerosis, SLE, ALS, IBD, focal segmental glomerulosclerosis, ankylosing spondylitis, gout, epilepsy, diabetes, psoriasis and/or an autoimmune disorder effecting the thyroid gland. Additional diseases include, those associated with the accumulation of misfolded proteins, idiopathic/cryptogenic diseases, diseases associated with inflammasome activation, Common variable immunodeficiency chronic fatigue syndrome, rheumatoid arthritis, lupus, migraines, multiple sclerosis and autism, ANCA-associated vasculitis (AAV), and/or immune-mediated inflammatory diseases (IMIDs).

In an embodiment, the treated cells are treated with one or more additional rounds with one or more of an RNase, a DNase, an antibody that binds a DNA and/or an antibody that binds an RNA, resulting in treated cells that more effectively treat a microorganism (a bacteria, a fungi, a protozoa, a virus, a cancer cell, a pre-cancer cell, a cell infected with a bacteria, a fungi, a protozoa, and/or a virus.

In an embodiment, treated cells treated with one or more rounds of a DNase, an RNase, an anti-DNA and/or an anti-RNA antibody are administered to a patient that receives a transplant of an organ, bone marrow, blood, muscle, skin, lymphoid tissue or other tissue. In an embodiment, the treated cells improve the efficiency, acceptance and survivability following a patient receiving a transplant of an organ, bone marrow, blood, muscle, skin, lymphoid tissue or other tissue.

In an embodiment, the treated cells are treated with one or more additional rounds with one or more of an RNase, a DNase, an antibody that binds a DNA and/or an antibody that binds an RNA, resulting in treated cells that more effectively treat a microorganism (a bacteria, a fungi, a protozoa, a virus, a cancer cell, a pre-cancer cell, a cell infected with a bacteria, a fungi, a protozoa, and/or a virus, wherein the treated cells are treated prior to, during or after a further treatment with a microbial cell a virus, a cellular component (including a cell membrane, organelles, lipids, proteins, cholesterol and/or LPS,), a eukaryotic cell, a tumor cell, a cancerous tissue, a mitogens (including ConA, PMA and/or ionomycin). These treated cells can be used for the treatment of chronic and acute bacterial, fungal, protozoan, viral infections, a microbial biofilm, a cancer (including a liquid and/or a solid cancer), an autoimmune disease.

In another embodiment, cells which were treated with multiple rounds of a DNase, an RNase, an anti-DNA and/or an anti-RNA antibody were used to manufacture biologic molecules. In an embodiment, the biologic molecules are a protein. In a further embodiment, the protein is an antibody, a peptide, an antibody fragment (including, an sfv, fv and/or Fab), an antibiotic, an anti-fungal an anti-viral, a food supplement and/or a biotherapeutic.

In another embodiment, cells which were treated with multiple rounds of a DNase, an RNase, an anti-DNA and/or an anti-RNA antibody were used to manufacture a biomolecule to be used in the food industry. The treated cells can be used for Precision Fermentation or PF to harness the power of the treated cells to optimize and efficiently generate proteins for use in the food industry. A protein that can be manufactured in the treated cells is chymosin, an enzyme (including amylase, a protease a lipase), a vitamin (including vitamin C and vitamin B12), amino acids (including those used as flavor enhancers, sweeteners, nutritional supplements and/or food additives). The treated cells can also be used to produce a pharmaceutical, a textile, a food ingredient, a fuel enzyme and/or cosmeceutical. In another embodiment, the treated cells can be used to manufacture cellulose, silk, chitin and alginate, each an alternative to a plastic.

In an embodiment, a treated cells is able to produce a biomolecule, including a protein or a peptide in a larger quantities, at a faster rate than a cell that is/are not treated with a DNase, an RNase, an anti-DNA antibody and/or an anti-RNA antibody. In an embodiment, a treated cell is/are able to grow at a faster rate than a cell that is not treated with a DNase, an RNase, an anti-DNA antibody and/or an anti-RNA antibody.

In another embodiment, a treated cell is/are able to produce a non-natural biomolecule (one not found naturally in nature), including a non-natural protein or a non-natural peptide at a faster rate than a cell that is not treated with a DNase, an RNase, an anti-DNA antibody and/or an anti-RNA antibody.

Cells that can be treated with a DNase, an RNase, an anti-DNA antibody and/or an anti-RNA antibody include Chinese Hamster Ovary (CHO) cells, human embryonic kidney (HEK) cells, S. cerevisiae, Penicillum spp, and hybridoma cells.

In a further embodiment, following treatment of cells with a DNase, an RNase, an anti-DNA antibody and/or an anti-RNA antibody, the treated cells are able to grow in different O₂, CO₂, temperature, UV environment. In an embodiment, following treatment of cells with a DNase, an RNase, an anti-DNA antibody and/or an anti-RNA antibody, the treated cells are able to grow at higher density and increase the amount of biomolecules produced. Other biomolecules that can be manufactured by cells treated with a DNase, an RNase, an anti-DNA antibody and/or an anti-RNA antibody include an antibody, an antibiotic, a cytokine a growth factor, a viral vector, an antigen, a viral vector (including an adeno-associated virus or AAV), a vaccine (including a protein vaccine and/or an mRNA and/or circular RNA vaccine), complex bivalent and trivalent biotherapeutics, a trivalent T-cell engager, a checkpoint modulator, a naive proteins, a recombinant protein, a vitamin, a hormone, a vaccine, and an antibody cytokine fusion biomolecule, a clotting and/or a coagulation factor, a TNF inhibitor, an Interferon, a monoclonal antibody, a receptor, a hormone (including a cortisol, estrogen, testosterone, thyroid hormone, growth hormone, insulin and melatonin).

In another embodiment, cells treated with a DNase, an RNase, an anti-DNA antibody and/or an anti-RNA antibody are able to metabolize and destroy xenobiotics, including those associated with pollutants in the water or earth (in dirt or soil) and heavy metals. Treated cells include a procaryotic treated cell that is/are used to improve the characteristics of a plant following administration of the treated cells to a soil or growth medium used for a plant nutrition. Treated cells are used in as part of a fermentation process (including, food and beverage fermentation to manufacture a cheese, a yogurt, a wine, and/or a beer).

In another embodiment, cells treated with a DNase, an RNase, an anti-DNA antibody and/or an anti-RNA antibody are used to manufacture materials used in the building industry, including a wood.

In an embodiment, cells treated with a DNase, an RNase, an anti-DNA antibody and/or an anti-RNA antibody are used to manufacture a honey. In an embodiment the treated cells are obtained from an insect, including a honey producing bee.

In a further embodiment, cells treated with a DNase, an RNase, an anti-DNA antibody and/or an anti-RNA antibody are used to manufacture food (feed) for animals, including livestock. Livestock include cows, sheep, goats, llamas, alpacas, horses and/or pigs. In a further embodiment, cells treated with a DNase, an RNase, an anti-DNA antibody and/or an anti-RNA antibody are used in aquaculture to manufacture biomolecules derived from plants. Treated cells can produce unique varieties of meat, milk, and fish. Currently unknown flowers on the planet, derived from cells as described above. Unique bouquets and landscapes, derived from plants as described above.

In an embodiment, treated cells, treated one or more times with an RNase, a DNase, an antibody that binds a DNA and/or an antibody that binds an RNA have higher resistance to variations of temperature and/or proteases. The variation of temperature can be an increase of one degree, two degrees, three degrees, four degrees, five degrees, six degrees, seven degrees, eight degrees, nine degrees, ten degrees, eleven degrees, twelve degrees, thirteen degrees, fourteen degrees, fifteen degrees, sixteen degrees, seventeen degrees, eighteen degrees, nineteen degrees, twenty degrees, twenty-one degrees, twenty-two degrees, twenty-three degrees, twenty-four degrees, twenty-five degrees, thirty degrees, thirty-five degrees, forty degrees, forty-five degrees, fifty degrees, fifty-five degrees, sixty degrees or more and each degrees in centigrade.

In an embodiment, cells treated with a DNase, an RNase, an anti-DNA antibody and/or an anti-RNA antibody are administered allogeneically or autogolously. In a further embodiment, treated cells have an altered expression of MHC, HLA, CD antigens. In another embodiment the treatment of the treated cells with a DNase, an RNase, an anti-DNA antibody and/or an anti-RNA antibody results in an altered interaction between the treated cells and anti-CD and anti-HLA antibodies. Such treated cells can be used to treat non-HLA matched transplant recipients.

In an embodiment, cells treated with a DNase, an RNase, an anti-DNA antibody and/or an anti-RNA antibody are administered to a patient as part of a vaccine preparation that also includes at least one of a nucleic acid derived from a bacteria (including, a nucleic acid of gram-negative bacteria, wherein, in an embodiment, the gram-negative bacteria is genera Pseudomonas.), and/or a nucleic acid derived from a fungi. In another embodiment, the treated cells that are part of a vaccine also comprise an adjuvant. In another embodiment, the treated cells that are part of a vaccine are administered to a patient one or more times.

In an embodiment, the treated cells are part of a vaccine that further comprises a nucleic acid associated with a cell surface membrane. The nucleic acid is comprised of a non-coding genetic segment and/or genetic segments that have prion like activity. In a further embodiment, the vaccine is prepared from a bacterial DNA or RNA. In a further embodiment, the vaccine is prepared from a bacterial nucleic acids associated with DNA or RNA associated with a cell surface membrane. In a further embodiment, the vaccine is prepared from a bacterial nucleic acids associated with DNA or RNA capable of triggering prionogenic aggregation of proteins. In another embodiment, the vaccine is prepared from a DNA or RNA gram-negative bacteria, wherein, in an embodiment, the bacteria genera Pseudomonas. In an embodiment, the vaccine is prepared from a fungal DNA or RNA.

In an embodiment, the treated cells that are part of a vaccine, wherein the vaccine is used for lifetime prolongation, the treatment age associated diseases, as a prophylactic to treat age associated diseases, as a prophylactic to treat an oncologic diseases, as a prophylactic to treat neurodegenerative diseases, and/or as a prophylactic to treat Alzheimer diseases.

In an embodiment, stem cells are treated with a DNase, an RNase, an antibody that binds to a DNA and/or an antibody that binds to an RNA, wherein the treated cells are administered to a patient as part of a bone marrow transplantation. In a further embodiment, the treated cells used for auto transplantation or allogeneic transplantation, are administered to a patient and the treated cells have greater survivability, lower immunogenicity, improved responsiveness and/or improved longevity of the transplant when compared to cells that are not treated with a DNase, an RNase, an antibody that binds to a DNA and/or an antibody that binds to an RNA.

In an embodiment, a cell is/are treated with a DNase, an RNase, an antibody that binds to a DNA and/or an antibody that binds to an RNA, and the treated cells are used for regrowth or repair of nervous tissue and nerve cells that have suffered damage. In an embodiment, cells are treated with a DNase, an RNase, an antibody that binds to a DNA and/or an antibody that binds to an RNA, are used to treat a patient that has suffered or suffers from a burn, an ulcer, a wound, physical damage and/or trauma. In an embodiment, cells treated with a DNase, an RNase, an antibody that binds to a DNA and/or an antibody that binds to an RNA, are used to regenerate cartilage, regenerate an organ or tissue and/or engineer an organ.

In an embodiment, cells treated with a DNase, an RNase, an antibody that binds to a DNA and/or an antibody that binds to an RNA, are used to generate improved red blood cells, white blood cells, platelets that are then administered to a patient. The improvement comprises one or more of a cell that lives longer after administration to the patient. For treated red blood cells, the improvement further comprises an increased ability to carry more oxygen than a non-treated red blood cell. For treated white blood cells, the improvement further comprises an increased ability to remove, block, kill, adhere to a foreign antigen that resides in the patient.

In an embodiment, cells treated with a DNase, an RNase, an antibody that binds to a DNA and/or an antibody that binds to an RNA, are white blood cells, dendritic cells, natural killer cells that following treatment no longer are specific to the antigen to which such cell had been responsive to. As a result, a B cell following treatment no longer produces an antibody for the specific antigen that the antibody produced by the B cell bound. Similarly, a T cell following treatment no longer produces a T cell receptor for the specific antigen that the T cell receptor produced by the T cell bound.

In an embodiment, a cell treated with a DNase, an RNase, an antibody that binds to a DNA and/or an antibody that binds to an RNA, is/are used to form a connective tissue, an epithelial tissue, a muscle tissue and/or a nervous tissue. In an embodiment, cells treated with a DNase, an RNase, an antibody that binds to a DNA and/or an antibody that binds to an RNA, produce greater amounts of collagen and/or hyaluronic acid.

In an embodiment cells treated with a DNase, an RNase, an antibody that binds to a DNA and/or an antibody that binds to an RNA, a cell is obtained from an embryonal cell, mesoderm, endoderm, ectoderm (including, stem cells, pluripotent stem cells), a red blood cell, an immune cell, a white blood cell, a leucocyte, a lymphocyte (T-cells, B-cells, NK cells, neutrophils, eosinophils, monocytes, basophils, macrophages), a CAR-T cell, a Platelet, a Nerve cell (e.g., neurons, glial cells, oligodendrocytes, astrocytes, microglial cells), an epithelial cell, a sensory epithelium, a fibroblast, a goblet cell, a Muscle cell, a Cartilage cell, a Bone cell, a Skin cell, an Endothelial cell, an Epithelial cell, a Fat cell, a muscle cell, a sensor cell, a pigment cell, a kidney cell, a placenta cell, a sex cell, a sperm, an egg, an ovary cell, a pre-malignant cell, a tumor cell, a cancer-associated cell (e.g. cancer associated fibroblasts), a fat cell, a circulating tumor cell, a neuroendocrine cell, an endocrine cell, a bone cell, a fat cells, a skin cell, an endothelial cell, a pancreatic cell, a plant cell, a seed coat, a Monocot cell, a dicot cell, a parenchyma cell a hematopoietic stem cell, an immune cell, a renal cell, a cancer, a sarcoma cell, a tissue and/or organ of an multicellular organism, a group of cells, an organ, organisms as well as a microorganism (including bacteria, fungi, and protists, microbiota, and/or viruses of all types, including bacteriophages), multicellular organs such as plant embryo, a terminally differentiated cholangiocyte or hepatocyte. In an embodiment, cells treated with a DNase, an RNase, an antibody that binds to a DNA and/or an antibody that binds to an RNA, are used for reprogramming and genome editing.

In an embodiment, cells treated with a DNase, an RNase, an antibody that binds to a DNA and/or an antibody that binds to an RNA, treated cells used for transplantation result in fewer adverse events and/or fewer complications following transplantation. In an embodiment, cells treated with a DNase, an RNase, an antibody that binds to a DNA and/or an antibody that binds to an RNA, result in fewer graft rejections and higher engraftment and/or higher efficacy of transplantation.

In an embodiment, white blood cells, fibroblasts, platelets, hematopoietic stem cells, red blood cells are treated with one or more rounds with one or more of a nuclease (including a DNase or an RNase) or an antibody that binds to a DNA and an antibody that binds to an RNA for the treatment or prevention of any solid tumor or liquid tumor.

In a further embodiment, white blood cells, fibroblasts, platelets, hematopoietic stem cells, red blood cells are treated with one or more rounds with one or more of a nuclease (including a DNase or an RNase) or an antibody that binds to a DNA and an antibody that binds to an RNA. The treated cells are then further treated with a microbial cell or the components of a microbial cell (including, LPS, particularly from Klebsiella), components of eukaryotic cells, tumor cells and/or organs and/or tissues or components of tumor cells and/or tissues and or organs.

In another embodiment, white blood cells, fibroblasts, platelets, hematopoietic stem cells, red blood cells are treated with one or more rounds with one or more of a nuclease (including a DNase or an RNase) or an antibody that binds to a DNA and an antibody that binds to an RNA. The treated cells are further treated with cells derived from an individual who is undergoing treatment for a cancer.

White blood cells treated with one or more rounds with one or more of a nuclease (including a DNase or an RNase) or an antibody that binds to a DNA and an antibody that binds to an RNA as part of the method for the creation and propagation of CAR-T cells. In an embodiment, the treated CAR-T cells are used to treat a metastasis, including a solid tumor or a liquid tumor.

In an embodiment, the treated cells are administered to an individual by either systemic or local administration. Such administration includes injection into the interior of a tumor, intrapleural, intraabdominal, administration into the wound site during/before/or after the removal of a tumor, and/or in a controlled release manner.

White blood cells, fibroblasts, platelets, hematopoietic stem cells, red blood cells, are from a mammal or a non-mammal, including cold-bloodied organisms.

In an embodiment, a white blood cell comprises a leucocyte, a lymphocyte (T-cells, a B-cell, an NK cell, a neutrophil, an eosinophil, a monocyte, a basophil, or a macrophage).

In an embodiment a treated cell is one that is used in biomanufacturing and/or the food industry.

In an embodiment, a treated cell is a eukaryotic cell (fungal, plant, mammalian, etc.), an organoid, a tissue, an embryo, an organ, a single-cellular organism or is derived or obtained from a multicellular organism.

In an embodiment, RBC Rh positive cells treated with a DNase, an RNase, an antibody that binds to a DNA and/or an antibody that binds to an RNA, lose their ability to interact and/or trigger the formation of anti-Rh antibodies. In a further embodiment, the RBC Rh positive treated cells can be used as part of a transplant and/or transfusion from an Rh-positive donor to an Rh-negative recipient without triggering an Rh-positive reaction.

In an embodiment, the treated cells are treated with one or more additional rounds with one or more of an RNase, a DNase, an antibody that binds a DNA and/or an antibody that binds an RNA are used to inhibit cancer and tumor growth.

In an embodiment, the treated cells are treated with one or more additional rounds with one or more of an RNase, a DNase, an antibody that binds a DNA and/or an antibody that binds an RNA are used to stop cancer and tumor growth.

In an embodiment, the treated cells are treated with one or more additional rounds with one or more of an RNase, a DNase, an antibody that binds a DNA and/or an antibody that binds an RNA are used to delay cancer and tumor growth.

In an embodiment, the treated cells are treated with one or more additional rounds with one or more of an RNase, a DNase, an antibody that binds a DNA and/or an antibody that binds an RNA result in cancer regression.

In a further embodiment, the treated cells are treated with one or more additional rounds with one or more of an RNase, a DNase, an antibody that binds a DNA and/or an antibody that binds an RNA are characterized by a higher product yield, higher expansion.

In a further embodiment, the treated cells are treated with one or more additional rounds with one or more of an RNase, a DNase, an antibody that binds a DNA and/or an antibody that binds an RNA have improved efficiency of transplantation and proliferation activity.

In an embodiment, the treated cells are treated with one or more additional rounds with one or more of an RNase, a DNase, an antibody that binds a DNA and/or an antibody that binds an RNA is a eukaryotic cell, a prokaryotic cells, single cell, a cell culture, an organoid, a 3D culture, a tissue, an embryo, an organ, a single-cellular organism, a microbiota.

In an embodiment, the treated cells are treated with one or more additional rounds with one or more of an RNase, a DNase, an antibody that binds a DNA and/or an antibody that binds an RNA are used to treat skin defects such as burns, ulcers, wounds.

In an embodiment, the treated cells are treated with one or more additional rounds with one or more of an RNase, a DNase, an antibody that binds a DNA and/or an antibody that binds an RNA are used for cartilage regeneration, organ regeneration, engineering organs.

In an embodiment, the treated cells are treated with one or more additional rounds with one or more of an RNase, a DNase, an antibody that binds a DNA and/or an antibody that binds an RNA are used for the transfusion to the individual

In an embodiment, the treated cells are treated with one or more additional rounds with one or more of an RNase, a DNase, an antibody that binds a DNA and/or an antibody that binds an RNA are used to be transferred to non-relative organisms,

In an embodiment, the treated cells are treated with one or more additional rounds with one or more of an RNase, a DNase, an antibody that binds a DNA and/or an antibody that binds an RNA have erased autoimmune memory.

In an embodiment, the treated cells are treated with one or more additional rounds with one or more of an RNase, a DNase, an antibody that binds a DNA and/or an antibody that binds an RNA produce compounds including peptides proteins, antibiotics, with higher activity against microorganisms (bacteria, fungi, protozoa, viruses, microbial biofilms), cancer cells, pre-cancer cells, cells infected with bacteria, fungi, protozoa, and viruses.

In an embodiment, the products produced by cells treated with one or more additional rounds with one or more of an RNase, a DNase, an antibody that binds a DNA and/or an antibody that binds an RNA used for the treatment of chronic and acute bacterial-fungal-protozoan-viral infections, microbial biofilms, cancers (liquid and solid), autoimmune diseases.

In an embodiment, cells and their products obtained without the use of mutagenesis or genome editing to change genome, and having properties that are currently absent in nature in similar and related organisms. In another embodiment, cells, organisms, Seeds, grains, animals, plants, and microorganisms and their products without changes to the genome and having properties currently absent in nature in similar and related organisms. In another embodiment, modern organisms exhibiting properties of their ancient ancestors with ability “dive back in time” millions of years ago. In another embodiment, cells or organisms obtained without changes to the genome and having properties of ancient ancestors, currently absent in nature in similar and related organisms. In another embodiment, products produced by organisms exhibiting properties of their ancient ancestors. In some embodiments these organisms are the source of plants including wood, flowers, fruits, vegetables, food products, medicinal herbs; vitamins; chemicals; pigments, dyes, medical drugs, psychoactive compounds, enzymes.

In some embodiments, the vaccine consists of extracellular DNA or RNA, including non-coding sequences, which does not penetrate cells and itself acts as an antigen, against which antibodies are produced. These antibodies then bind to the extracellular DNA and RNA in the body, preventing their interaction with extracellular proteins that lead to diseases and aging. In some embodiments, the vaccine consists of microbial DNA or RNA.

### EXAMPLES

### Example 1. Effect of Treatment of Immune Cells on Different Components of their Anticancer and Antimicrobial Activity

We evaluated the phagocytic activity of immune cells using FITC-stained E. coli, adjusted to a specific concentration after cultivation and labeling. Macrophages or neutrophils, isolated by gradient centrifugation, were seeded in multi-well plates and incubated with these bacteria to assess phagocytosis. Post-incubation, undigested bacteria were washed off, and bacterial uptake was quantified via fluorescence. Phagocytosis efficiency was analyzed by comparing fluorescence in treated samples to controls.

Additionally, we studied phagocytic responses in granulocytes and agranulocytes, treated with an RNase and a DNase, and measured Myeloperoxidase (MPO), Neutrophil Acid Phosphatase (NACP), and Neutrophil Alkaline Phosphatase (NAP) activities:

MPO Activity: Measured by the color change of o-dianisidine dihydrochloride in the presence of hydrogen peroxide, and quantified spectrophotometrically at 460 nm.

NACP Activity: Assessed by hydrolysis of para-nitrophenyl phosphate to para-nitrophenol, monitored spectrophotometrically at 405 nm.

NAP Activity: Determined by fluorescence of 4-methylumbelliferyl phosphate at 365/445 nm excitation/emission.

These methods provided insights into the phagocytic functions and enzymatic activities of different immune cell groups under various conditions.

TABLE 2
Phagocytosis activity.

| granulocytes control | granulocytes treated |
|---|---|
| 76 ± 5 | 95 ± 4* |

| agranulocytes control | agranulocytes treated |
|---|---|
| 69 ± 6 | 92 ± 5* |

*p < 0.05

The data shown in Table 2 shows that the treated cells possess significantly higher phagocytosis activity.

TABLE 3

| | MPO | NAP | NA_{C}P |
|---|---|---|---|
| Groups | ng/mL | ng/mL | u.c. |
| SL4-C | 205 ± 17 | 70 ± 12 | 1239 ± 384 |
| SL4-T | 365 ± 28* | 164 ± 22* | 1920 ± 243* |

*p < 0.05

As shown in Table 3, the enzymatic activities of MPO, NACP, and NAP are elevated in treated cells when compared to a control.

### Example 2. Effect of Treated Cells on Cancer Cell Lines

We investigated the anticancer effects of treated SL4-T cells on tumor cells. SL4-C and SL4-T cells, derived from whole blood, buffy coats, leukapheresis, Filgrastim or Plerixafor, Activation of WBCs utilized a different DNase, RNase, including S1 and Micrococcal nuclease to target G-quadruplexes and Z-DNA. We co-cultured SL4 cells with the NCL-H1299 lung carcinoma and T98G glioblastoma cell lines in RPMI 1640.

Cultivation involved growing H1299 cells in 96-well plates and T98G cells in 25 cm2 flasks, both in respective media supplemented with 10% FBS. Cell viability was measured after exposing cancer cells to SL4-based treatments for 10 minutes to 24 hours, comparing absorbance with control wells to determine effectiveness.

Results showed SL4-T cells, significantly enhanced anticancer activity against tumor cells, with efficacy data presented in Table 1.

TABLE 4
Anticancer activity of treated cells.

| | Average Viability |
|---|---|
| | [%] for H1299 |
| Probe | cells and T98G |
| Untreated control | 100 |
| Granulocytes (untreated) | 97.1 ± 8.7 |
| Agranulocytes (untreated) | 96.5 ± 5.3 |
| Granulocytes + platelets (untreated) | 95.3 ± 4.1 |
| Agranulocytes + platelets (untreated) | 96.8 ± 9.4 |
| Granulocytes + Agranulocytes + platelets (untreated) | 91.7 ± 3.5 |
| White blood cells (untreated) | 97.0 ± 4.5 |
| White blood cells + platelets + red blood cells (untreated) | 92.9 ± 6.6 |
| Mesenchymal stem cells (untreated) | 94.5 ± 4.0 |
| Granulocytes (treated) | 53.2 ± 7.1* |
| Agranulocytes (treated) | 64.8 ± 7.3* |
| Granulocytes + platelets (treated) | 24.2 ± 5.5* |
| Agranulocytes + platelets (treated) | 19.9 ± 6.3* |
| Granulocytes + Agranulocytes + platelets (treated) | 8.1 ± 3.9* |
| White blood cells (treated) | 12.5 ± 8.2* |
| White blood cells + platelets + red blood cells (treated) | 9.5 ± 3.4* |
| Mesenchymal stem cells (treated) | 35.0 ± 6.1* |
| Granulocytes + Agranulocytes + platelets (treated) | 14.5 ± 7.1* |
| White blood cells + platelets + red blood cells (treated) | 11.3 ± 4.5* |
| Granulocytes + Agranulocytes + platelets (treated with S1 nuclease) | 5.0 ± 1.2* |
| Granulocytes + Agranulocytes + platelets (treated with Micrococcal nuclease) | 4.1 ± 0.8* |

*p < 0.05

### Example 3. Effect of Treated Cells or their Products in Mouse Models of Solid Tumors

### Spontaneous Breast Cancer Model

The SL4-T were obtained as previously described.

### Animal Models:

aged SWISS mice, specifically females within a defined weight and age range, are employed in the experiments, housed, and cared for under specific conditions as per standardized guidelines.

### Tumor Detection and Measurement:

Spontaneous mammary tumors were detected via palpation and monitored through caliper measurements taken weekly.

Mice were treated with SL4-C(10\hat{ }7 cells/injection) and SL4-T (10\hat{ }7 cells/injection) weekly, by i.v. injection.

TABLE 5
Spontaneous mammary tumors size reduction percentage relative to
day −1 (one day before injection) after SL4-C and SL4-T treatment.

Tumor size

| Mice groups | 1^{st} day before 1 injection | 5 injections |
|---|---|---|
| SL4-C | 0.0 | +127.33 ± 15.57 |
| SL4-T | 0.0 | −58.3 ± 7.9* |

*p < 0.05 compared with SL4-C and untreated control

The cytokines were measured by ELISA kits. The plasma tumor necrosis factor alpha (TNF-a) level was measured using eBioscience® ELISA kits (Affimetrix, Santa Clara, USA).

TABLE 6

| Mice | | | | | | | | | |
|---|---|---|---|---|---|---|---|---|---|
| groups | IFNy | IL - 1a | IL - 4 | IL - 7 | IL - 9 | IL - 12 | IL - 15 | IL - 17 | TNFa |
| Healthy | 3.2 ± 0.2 | 58.0 ± 1.0 | 0.3 ± 0.1 | 5.6 ± 0.2 | 162.4 ± 2.5 | 12.3 ± 0.5 | 47.9 ± 0.3 | 3.1 ± 0.2 | 6.3 ± 0.8 |
| Control | 12.5 ± 0.3 | 43.2 ± 1.9 | 0.2 ± 0.1 | 4.4 ± 0.9 | 160.1 ± 10.2 | 11.7 ± 1.3 | 38.4 ± 2.5 | 2.6 ± 0.8 | 76.8 ± 3.5 |
| SL4-T | 58.8 ± 3.3* | 139.8 ± 14.4* | 4.4 ± 0.7* | 28.7 ± 2.3* | 161.2 ± 13.8 | 28.9 ± 3.2* | 149.1 ± 10.7* | 74.6 ± 6.2* | 165.5 ± 6.1* |

*p < 0.05

The disparate cytokine profiles are set forth in Table 6 and show post and SL4-T treatment which underscore potential immunomodulatory properties, where SL4-T particularly emerges as a potent immunostimulant. Elevated cytokines pivotal for antitumor responses (like IFNγ and IL-12) post-SL4-T treatment flag its prospective utility in harnessing and enhancing immune responses against tumors. The ability to significantly modulate cytokine profiles indicates potential applications in contexts requiring immunomodulation, such as cancer, infections, or immune disorders.

Additionally, MRC fibroblasts were treated using multiple rounds of nuclease treatment, as described earlier. These cells were administered in various combinations and dosages to different mouse groups with spontaneous breast cancer.

We next studied different routs of SL4-T administration as set forth in the Table below.

TABLE 7

| | | Tumors size changes on day 30 |
|---|---|---|
| Group | Route | compared to day −1 |
| Control | n/a | +71.9 ± 9.6% |
| SL4-T | intraperitoneal | −81.0 ± 13.2%* |
| SL4-T | intratumorally | −88.5 ± 9.3%* |
| SL4-T | subcutaneous | −49.7 ± 8.3 |
| SL4-T | intramuscular | −47.2 ± 10.5 |
| MRC-T | i.v. | +4.5 ± 4.0* |
| SL4-T + MRC-T | i.v. | −87.5 ± 8.7* |
| SL21-Tn** | 60 | −90.4 ± 9.0* |
| SL21-Ta** | 60 | −85.6 ± 8.4* |

**Tn - granulocytes, agaranulocytes and platelets treated with nucleases
**Ta - granulocytes, agaranulocytes and platelets treated with antinucleic acid antibodies

### Pancreatic Cancer Model

We investigated the efficacy of various types of treated cells and their supernatants/products in treating advanced pancreatic cancer. The SL4-T cells were procured as previously outlined. The supernatants/products derived from these cells, referred to as P-SL, were obtained following the procedure described subsequently. To prepare P-SL 1 ml of white blood cells were combined with 1 ml of LPS at a final concentration of 5 ng/ml and incubated for 60 minutes at 37° C. Subsequently, the suspension was centrifuged at 3000 rpm, the supernatant was collected and passed through a 0.22 μm filter.

After activation, we obtained the P-SL by filtration, then to eliminate the possibility of LPS remaining in the obtained filtrate (directly in the P-SL4), polymyxin B was added to a series of samples at a final concentration of 1 μg/ml. The supernatant was incubated for 60 minutes at 37° C., 5% CO₂.

### PANC-1 Cell Cultivation and Transplantation Summary:

Cultivation: PANC-1 cells were grown in 25 cm2 flasks for adherent cultures, subcultured every 3 days using trypsin-EDTA, and maintained at 37° C. in 5% CO₂. Viability was assessed by MTT, requiring at least 90% viability.

Medium: Cultured in DMEM with 10% FBS. For transplantation, cells from passages 5-10 were used.

Preparation: Cells in exponential growth were trypsinized, centrifuged, counted, and resuspended in serum-free DMEM at 5×10\hat{ }7 cells/ml for transplantation.

Transplantation: For subcutaneous injections into mice we used an insulin syringe with a 27G needle, inserting intradermally at the withers. Cell suspension (5×10\hat{ }6 cells in 100 μl DMEM) is injected slowly to form a papule, ensuring no leakage.

Animal Model: Female C57BL/6 mice received 3×10\hat{ }6 PANC-01 cells in Matrigel flank injections. Treatment began when tumors reached ˜100 mm³.

This concise method outlines the cultivation, preparation, and transplantation techniques for PANC-1 cells into mice for cancer research.

TABLE 8
Treatment groups.

| | | Intraperitoneal injection | |
|---|---|---|---|
| Mice groups | Agent | volume, ml | Cell count |
| Control | DMEM | 0.200 | |
| SL4-C | SL4-C | 0.200 | 10⁷/ml |
| SL4-T | SL4-T | 0.200 | 10⁷/ml |
| P-SL4-C | SUPERNATANT | 0.200 | SUPERNATANT isolated from SL4- |
| | FROM SL4-C | | C 10⁷/ml |
| P-SL4-T | SUPERNATANT_{—} | 0.200 | SUPERNATANT isolated from SL4- |
| | T FROM | | T 10⁷/ml |
| | SL4-T | | |

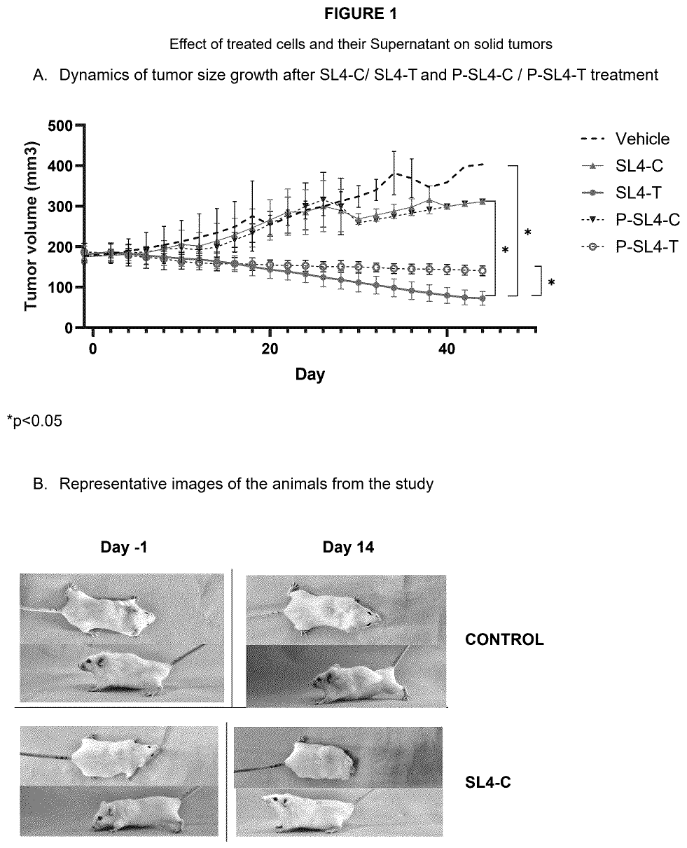

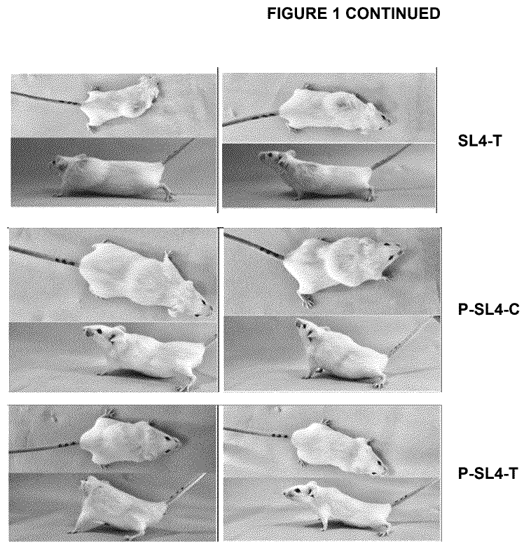

Data received are presented in FIG. 1.

Compared to SL4-C, the SL4-T revealed a reduction in the tumor size. Similarly, while P-SL4-C displayed an increase in tumor size. P-SL4-T demonstrated a notable reduction in tumor size, similar to that of the performance of SL4-T against solid tumors.

### Example 4. The Use of Treated Cells for the Treatment of Solid Tumors

### Ethical Consideration and Compliance

Patient data has been anonymized and/or utilized with explicit patient consent for research and publication. Adherence to relevant ethical guidelines and regulatory compliance.

Patient #1 Male, 74 y.o. Lung Cancer, Stage IIIB. T4N2M0. G3. Large-cell carcinoma. Previously treated with chemotherapy (Etoposide+Cisplatin), 3 cycles, without effect, tumor growth persisted.

SL4-T i.v. allogeneic and autologous, weekly or daily, intermittent course, from 10\hat{ }6 to 10\hat{ }9 per injection.

The decrease of the largest diameter is 17% with no new lesions, refers to Stable Disease (SD) according to RECIST 1.1.

Patient #2 Male, 85 y.o Squamous Cell Carcinoma of the oral cavity floor, T4NOMO, G2-G3.

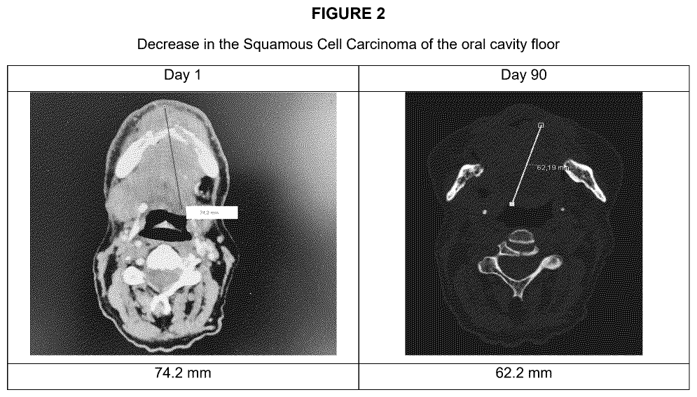

SL4-T i.v. allogeneic and autologous, weekly or daily, intermittent course, from 10\hat{ }6 to 10\hat{ }9 per injection. The results are presented in FIG. 2.

The decrease of the largest diameter is 16.2% with no new lesions, refers to Stable Disease (SD) according to RECIST 1.1.

Patient #3 Male, 80 y.o. Adenocarcinoma of the right lung S6, pleural carcinomatosis. The patient underwent six cycles of chemotherapy utilizing a combination of Carboplatin and Pemetrexed, followed by maintenance therapy with Pembrolizumab.

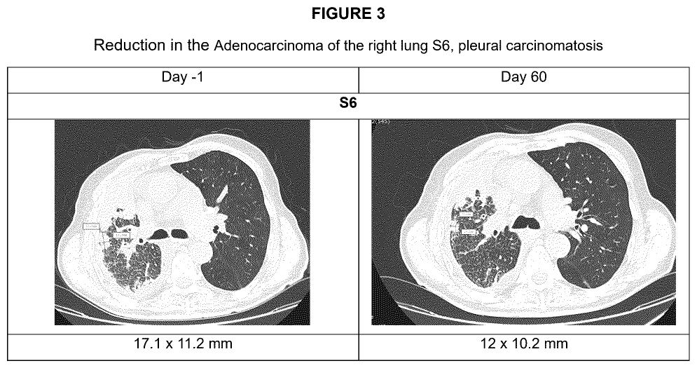

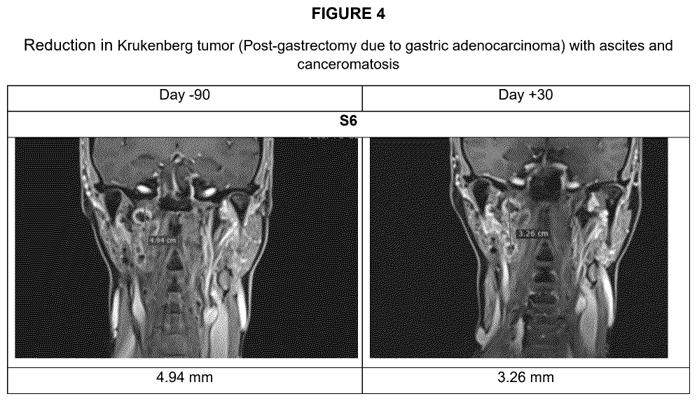

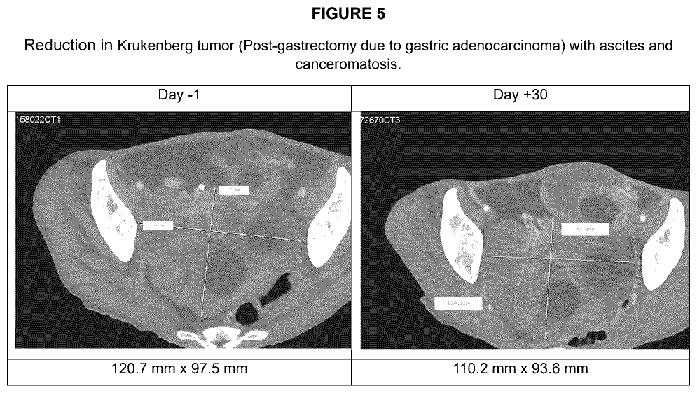

SL4-T i.v. allogeneic and autologous, Intravenous-Every 3 days; Intrapleural—Weekly for three times, from 10\hat{ }6 to 10\hat{ }9 per injection. The results are shown in FIG. 3.

TABLE 10
Analysis of pleural fluid cytology.

| | Day 21 |
|---|---|
| | After 3^{rd} |
| Day −1 | intrapleural injection |
| Lung adenocarcinoma TTF-1, Napsin - A +, | NO tumor cells |
| D2-40 − negative. | |

Following another 60 days of therapy, the needle biopsy confirmed that the original tumor site was fibrosed, highlighting the complete response.

Patient #4 Female, 56 y.o. Adenoid cystic carcinoma (ACC) of the right parotid gland. The patient underwent chemo- and proton beam radiation therapy.

SL4-T i.v. allogeneic and autologous, Intravenous-Every 3 days; Intrapleural—Weekly for three times, from 10\hat{ }6 to 10\hat{ }9 per injection.

The decrease of the largest diameter is 34.0% with no new lesions, refers to Partial Response (PR) according to RECIST 1.1.

Patient #5 Female, 40 y.o. Krukenberg tumor (Post-gastrectomy due to gastric adenocarcinoma) with ascites and canceromatosis.

SL4-T i.v. allogeneic and autologous, Intravenous-Every 3 days; Intrapleural—Weekly for three times, from 10\hat{ }6 to 10\hat{ }9 per injection.

The decrease of the largest diameter is 7.0%* with no new lesions, refers to Stable Disease (SD) according to RECIST 1.1.

TABLE 11
Dynamics of patient's weight

| Day −1 | Day 30 |
|---|---|
| 36 kg | 41 kg |

Patient #6 Male, 6 y.o. Bilateral Adrenal Neuroblastoma, Malignant neoplasm of retroperitoneum (C48.0).

Stage 4. Chemo- and immunotherapy failed.

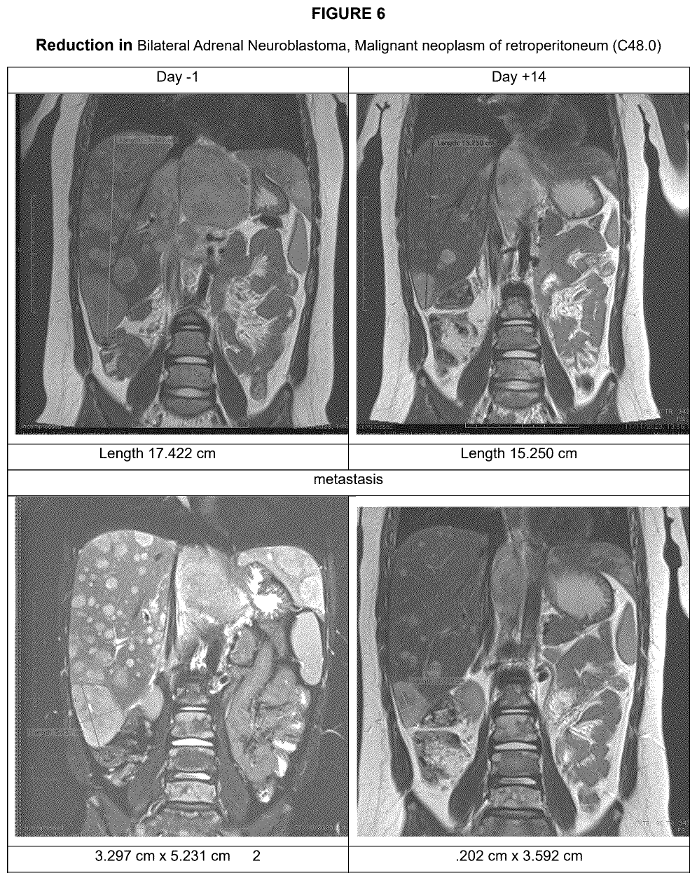

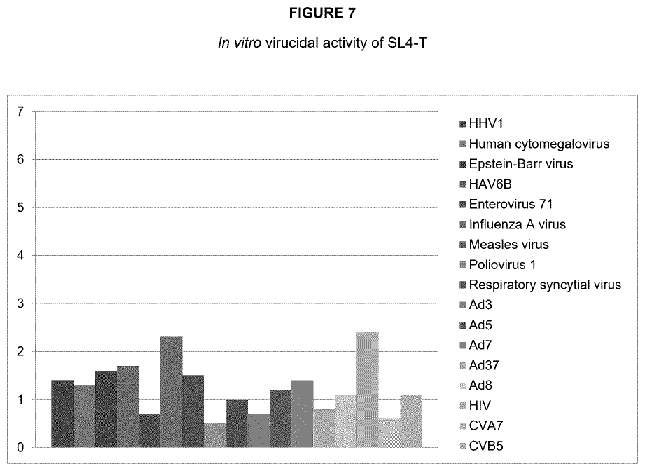

SL4-T i.v. allogeneic and autologous, Intravenous-Every 3 days; Intrapleural—Weekly for three times, from 10\hat{ }6 to 10\hat{ }9 per injection. The results are shown in FIG. 6.

TABLE 12
Bloodwork

| | | | | | | Normal |
|---|---|---|---|---|---|---|
| Test | Day −10 | Day −7 | Baseline | Day +7 | Day +20 | value |
| PLT | 186 × | 162 × | 121 × | 196 × | 301 × | 150-380 |
| | 10 * 3/μL | 10 * 3/μL | 10 * 3/μL | 10*3/pL | 10 * 3/μL | |
| RBC | 3.73 × | 3.57 × | 3.44 × | 3.86 × | 3.69 × | 4.00-5.40 |
| | 10 * 6/μL | 10 * 6/μL | 10 * 6/μL | 10 * 6/μL | 10 * 6/μL | |
| LEU | 5.69 × | 4.44 × | 3.44 × | 5.57 × | 5.14 × | 4.5-11.0 × |
| | 10 * 3/μL | 10 * 3/μL | 10 * 3/μL | 10 * 3/μL | 10 * 3/μL | 10 * 3/mm3 |
| Hemoglobin | 97 | 92 | 78 g/dL | 107 g/dL | 104 g/dL | 13.0-18.0 |
| | | | | | | g/dL |
| AST | 268 U/L | 342 U/L | 882 U/L | 75 U/L | 42 U/L | 8.0-48.0 |
| | | | | | | U/L |
| ALT | 52 U/L | 55 U/L | 50 U/L | 31 U/L | 23 U/L | 7.0-55.0 |
| | | | | | | U/L |
| LDH | 5365 U/L | 5710 U/L | 8529 U/L | 4392 U/L | 1125 | <270 U/L |
| CRB | 17 mm/h | 23 mm/h | 26 mm/h | 3 mm/h | 1.6 mm/h | 0-15 mm/h |
| Creatin | 423 U/L | 471 U/L | 439 U/L | 130 U/L | 55 U/L | 39-308 U/L |
| phosphokinase | | | | | | |
| Creatinine | 0.54 mg/dL | 0.61 mg/dL | 0.52 mg/dL | 0.57 mg/dL | 0.56 mg/dL | 0.6-1.3 mg/dL |
| NSE | >740 ng/ml | — | >740 ng/ml | 237 ng/ml | 37 ng/ml | <16.3 (ng/ml) |

The decrease of the largest diameter is 7.0% with no new lesions, refers to Stable Disease (SD) according to RECIST 1.1.

During the treatment course, the patient reported a gradual alleviation in weakness and an enhancement in overall vitality.

Notable decrease in the frequency and intensity of chest pain and improvement in breathing capacity during activities.

Appetite incrementally improved, and weight stabilized, suggesting a possible reversal in cachexia.

Reduction in the incidence of night sweats and no further episodes of fever were reported.

### Results:

CT scans after 3 months demonstrated a shrinkage of tumor size with no evident progression or new metastatic formations.

### Symptomatic Changes:

Remarkable improvement in quality of life, reduced symptom burden, and augmented daily functional capacity.

The symptom alleviation and stabilization suggests clinical utility of allogeneic and autologous SL4-T in the management of cancers including those unresponsive to available therapies.

### Example 5: Effect of Treatment on Generating Autologous Cells with Enhanced Anticancer Activity

Female NOD SCID mice were implanted with Raji cells and treated with human CD19 CAR T cells, either untreated or modified with DNase/RNase (CAR-Tn) or benzonase (CAR-Tb) treatments. CAR-T cell treatment started 8 days post-implantation, with tumor volume measured at day 30 (Table 13).

TABLE 13
Effect of tested products on blood cancers.

| | Tumor size related to |
|---|---|
| Group | untreated control |
| Untreated control | 100% |
| CAR-T control | 47 ± 13% |
| CAR-Tn | 0* |
| CAR-Tb | 0* |

*p < 0.05 compared to CAR-T control

These data surprisingly demonstrate that treated CAR-T cells exhibit a high level of anticancer activity.

We next evaluated how the production of CAR-T cells from treated cells would affect their activity.

For that we T-cells which were treated from 3 to 12 cycles of DNase and RNase at 40 μg/ml were further used for the production of CD19 CAR-T as previously described (Wang X, Rivière I. Clinical manufacturing of CAR T cells: foundation of a promising therapy. Molecular Therapy-Oncolytics. 2016 Jan. 1; 3).

After that the resulted cells were used to treat female NOD SCID (CB17-Prkdescid/NcrCrl) mice on day 8 after subcutaneously implantation of 7.0 log 10 Raji cells (Table 14).

TABLE 14
The utilization of treated cells in the generation of CAR-T cells.

| | Tumor size related to |
|---|---|
| Group | untreated control |
| Untreated control | 100% |
| CAR-T control | 44 ± 11% |
| CAR-T made from treated WBC | 0* |

*p < 0.05 compared to CAR-T control

These data surprisingly demonstrate that the use of treated cells for the production of CAR-T cells results in the formation of cells with a high level of anticancer activity.

Additionally, we investigated the potential of treated tumor-infiltrating lymphocytes (TILs) from pancreatic adenocarcinoma. TILs were expanded and either left untreated (TIL-C) or treated with DNase and RNase at 1 μg/mL (TIL-T1, TIL-T2), stimulated with anti-CD3, and co-cultured with irradiated PBMCs. The efficacy of TILs was assessed by IFN-γ levels after co-cultivation with a pancreatic cancer cell line.

This study evaluates the impact of nuclease treatment on CAR-T cells and TILs against cancer, with findings suggesting enhanced anticancer activity (Table 15).

TABLE 15
Effect of Cell Treatment on Their Anticancer Responses.
The data clearly show that treatment of immune cells resulted
in a significantly higher anticancer response.

| TIL-C | TIL-T1 | TIL-T2 |
|---|---|---|
| 140 ± 18 | 376 ± 112* | 521 ± 101* |

*p < 0.05

### Example 6. The Use of Tuned Cells for Cancer Prophylaxis and Post-Surgical Treatment

A 58-year-old patient was diagnosed with a single colorectal polyp, accompanied by an elevation of her carcinoembryonic antigen (CEA) levels to 22.1 ng/ml, significantly higher than the normal CEA values (<4.3 ng/ml). Following laparoscopic removal of the polyp, the patient underwent therapy involving daily intravenous injections of SL4-T cells for 7 days. After the therapy concluded, the CEA level decreased to 2.5 ng/ml. The patient's CEA levels were monitored every six months through blood tests. Twelve months post-therapy, the CEA levels remained within the normal range.

The data received indicate that the use of tuned SL4-T cells can be effective for the prophylaxis of cancer as well as in managing patients post-surgically after the removal of neoplasms.

### Example 7. The Use of Treated Cells in Combination with Other Anticancer Therapies

A 7-year-old child was diagnosed with bilateral neuroblastoma with involvement of both right and left adrenal glands), multiple skeletal bone lesions, liver involvement, bone marrow involvement. Stage IV, T3N×Mx, that showed poor response to standard chemotherapy regimens (Temozolomide, 200 mg per os and Irinotecan, 50 mg, i/v).

SL4-T could potentiate the effects of chemotherapy and mitigate its side effects. Administered at a dose of 10\hat{ }8 cells/ml daily i/v for the first four weeks, SL4-T was closely monitored for its impact on the child's health and ameliorating the severity of chemotherapy.

Over the course of 2 months, remarkable improvements were observed. The size of the neuroblastoma tumor reduced by 60%, a significant reduction that was corroborated by MRI scans. Furthermore, the levels of tumor markers in the blood decreased by 150% from their baseline levels, indicating a substantial decline in the presence of cancerous cells (Table 1).

The side effects profile before and after the introduction of SL4-T illustrated a dramatic change in the child's experience of chemotherapy. Nausea and vomiting, previously reported at a frequency of 90%, dropped to 30%. Other severe side effects such as neutropenia, skin lesions, and fatigue saw similar declines in frequency, making the chemotherapy process considerably more tolerable for the young patient. Notably, even the more common side effects like hair loss, mucositis, and anemia were significantly reduced, further evidence of the positive impact of SL4-T on mitigating the negative aspects of cancer treatment (Table 16).

TABLE 16
Neuroblastoma size and tumor markers levels reduction.

| | | | |
|---|---|---|---|
| | | 1 day before 1^{st} SL4-T administration, | 30 days after |
| | 30 days before 1^{st} SL4-T | after 2 courses | SL4-T |
| Parameter | administration | Temozolomide + Irinotekan | treatment |

Tumor control check

| | | | |
|---|---|---|---|
| S6, liver | 45 × 28 | 52 × 33 | 35 × 22 |

Tumor markers

| | | | |
|---|---|---|---|
| NSE [ng/ml] | 680 | 740 | 21.8 |
| Ferritin | 240.3 | 893.8 | 151.1 |
| [μg/ml] | | | |

TABLE 17
Side effects during chimiotherapy + SL4-T.

| | Temozolomide + | Temozolomide + |
|---|---|---|
| Side Effect | Irinotekan | Irinotekan + SL4-T |
| Nausea and Vomiting | 4 | 0 |
| Neutropenia | 3 | 0 |
| Skin Lesions | 1 | 0 |
| Fatigue | 4 | 1 |
| Loss of Appetite | 4 | 0 |
| Diarrhea | 3 | 0 |
| Nasal Congestion | 2 | 0 |
| Headaches | 3 | 1 |
| Abdominal Pain | 2 | 0 |
| Weight Loss | 2 | 0 |
| Anemia | 3 | 0 |
| Infections | 2 | 0 |
| Hair Loss | 3 | 0 |
| Mucositis | 2 | 0 |

Scoring system:
0-None;
1-Mild;
2-Moderate;
3-Severe;
4-Very severe

### Example 8. The Effect of Antimicrobial and Anticancer Products Produced by Treated Cells

We analyzed the activity of products and supernatants from SL4-C and SL4-T, which are referred to as P_4C and P_4T, respectively. Also, some cells were treated with anti-DNA and anti-RNA antibodies SL4-Ta Supernatants from SL4-C and SL4-T were collected, and protein levels were equalized between the samples. This was done by first determining the protein amount using the Bradford assay, followed by quantification with Coomassie brilliant blue dye binding.

TABLE 17
Antibacterial activity of products obtained
from SL-4C and SL-4T against bacteria.

| | Maximum dilution of the Products | | |
|---|---|---|---|
| | sufficient to achieve MIC | | |
| Tested strains | P_4C | P_4T | P_4Ta |
| Staphyloccous aureus | 1/2 | 1/8 | 1/8 |
| ATCC 29213 | | | |
| Staphyloccous aureus | na | 1/8 | 1/4 |
| VT 209R | | | |
| Staphylococcus aureus | na | 1/8 | 1/8 |
| MRSA VT 4490 | | | |
| Bacillus pumilus | 1/2 | 1/4 | 1/2 |
| VT 1278 | | | |
| Enterococcus faecalis | na | 1/8 | 1/4 |
| ATCC 29212 | | | |
| Pseudomonas aeruginosa | na | 1/4 | 1/4 |
| ATCC 27853 | | | |
| Escherichia coli | na | 1/8 | 1/8 |
| ATCC 25922 | | | |
| Klebsiella pneumoniae | na | 1/4 | 1/4 |
| VT 1698 | | | |
| Candida albicans | na | 1/4 | 1/2 |
| ATCC 90028 | | | |
| Aspergillus niger | na | 1/8 | 1/8 |
| VT 1591 | | | |
| Mycobacterium smegmatis | na | 1/4 | 1/4 |
| ATCC 607 | | | |

na - not active

Across all microbial strains, P_4T/Ta consistently demonstrated superior inhibitory effects.

### Activity of Products Secreted by Cells of Various Organisms

We analyzed the products secreted by white blood cells against microorganisms using multidrug resistant strain of Pseudomonas aeruginosa MR45

Prestimulation was done by adding bacterial LPS to WBC for 45 min.

TABLE 18
Antimicrobial effect of products against P. aeruginosa.

| | | Maximum dilution of the |
|---|---|---|
| | | Product sufficient to achieve |
| N | Source of Product | MIC |
| 1 | hu SL-4C -human WBCs | na |
| 2 | hu SL-4T - human WBCs | 1/2 |
| 3 | ps_hu SL-4C- WBCs pre-stimulated with LPS for | 1/4 |
| | human cells | |
| 4 | ps_hu SL-4T - WBCs pre-stimulated with LPS for | 1/64 |
| | human cells | |
| 5 | eqSL-4C-horse WBCs | 1/4 |
| 6 | eqSL-4T - horse WBCs | 1/8 |
| 7 | ps_eqSL-4C- WBCs pre-stimulated with LPS from | 1/8 |
| | horse | |
| 8 | ps_eqSL-4T - WBCs pre-stimulated with LPS from | 1/256 |
| | horse | |
| 9 | cowSL-4C -cow WBCs | na |
| 10 | cowSL-4T -cow WBCs | 1/2 |
| 11 | ps_cowSL-4C- WBCs pre-stimulated with LPS from | 1/4 |
| | cow | |
| 12 | ps_cowSL-4T - WBCs pre-stimulated with LPS from | 1/32 |
| | cow | |
| 13 | pigSL-4C-pig WBCs | na |
| 14 | pigSL-4T -pig WBCs | 1/2 |
| 15 | ps_pigSL-4C- WBCs pre-stimulated with LPS from | 1/4 |
| | pig | |
| 16 | ps_pigSL-4C- WBCs pre-stimulated with LPS from | 1/16 |
| | pig | |

Data received demonstrate that the treated cells of different organisms following the activation produce highly active Products.

### Production of Products Using Different Subsets of Cells

The study presented showcases the production and efficacy of Products, utilizing different activators across various cellular fractions. The main aim was to identify the optimal combinations that offer the highest antimicrobial efficacy, using the test strain Aspergillus niger.

Granulocyte and agranulocyte fractions were obtained from buffy coats using gradient centrifugation at densities of 1.077 and 1.119.

SL-4C/SL-4T—the whole subset of all WBC and platelets.

TABLE 17
Antimicrobial effect of products against Aspergillus spp

| | | Maximum dilution |
|---|---|---|
| | | of the Product |
| | | sufficient to |
| | source of Product | achieve MIC |
| 1 | The whole subset of all WBC and platelets | na |
| 2 | The whole subset of all WBC and platelets | 1/4 |
| 3 | granulocytic fraction | na |
| 4 | granulocytic fraction treated with nuclease treatment | 1/2 |
| 5 | agranulocytic fraction | na |
| 6 | agranulocytic fraction treated with nuclease | 1/2 |
| | treatment | |
| 7 | granulocytic fraction + platelets | na |
| 8 | g^{x} + platelets | 1/2 |
| 9 | granulocytic fraction + platelets treated with | na |
| | nuclease treatment | |
| 10 | granulocytic fraction treated with nuclease treatment + | 1/4 |
| | platelets treated with nuclease treatment | |
| 11 | agranulocytic fraction + platelets | na |
| 12 | agranulocytic fraction treated with nuclease | 1/2 |
| | treatment + platelets | |
| 13 | agranulocytic fraction + platelets treated with | 1/2 |
| | nuclease treatment | |
| 14 | agranulocytic fraction treated with nuclease | 1/4 |
| | treatment + platelets treated with nuclease treatment | |

We next analyzed the effect of bacterial stimulators on SL-4C and SL-4T, where we showed that the pretreating of the SL-4T with Klesiella spp significantly increased their antimicrobial activity. We marked prestimulated cells as “ps”.

TABLE 18
Inhibition of biomass S. aureus biofilms (24 h and 48 h- old)

| | | P_4C | P_4T | psP_4C | psP_4T | P_4C | P_4T | psP_4C | psP_4T |
|---|---|---|---|---|---|---|---|---|---|
| Biofilm | age of the | 24 h | 24 h | 24 h | 24 h | 48 h | 48 h | 48 h | 48 h |
| | formed | | | | | | | | |
| | biofilm | | | | | | | | |

| | |
|---|---|
| Tested | S. aureus VT 104 |
| strain | |

| | | | | | | | | |
|---|---|---|---|---|---|---|---|---|
| Biomass inhibition, | −23 ± 3.9 | −43 ± 7.0 | −25 ± 6.9 | −89 ± 5.9 | −16 ± 4.5 | −67 ± 8.4 | −34 ± 2.1 | −92 ± 5.9 |
| % | | — | | | | | | |
| Log₁₀ CFU/ml | 8.61 | 3.51 | 6.90 | 2.06 | 8.78 | 2.65 | 5.82 | 1.38 |

Data received in Table 18 highlights that the antimicrobial activity of Products can be increased in treated cells following their prestimulation.

Efficacy of Products from Treated Cells on Cancer Cell Viability

The study aimed to evaluate the effect of various Products on the viability of the H1299 and T98G cancer cell lines (Results set forth in Table below).

TABLE 19

| | | The percentage | The percentage |
|---|---|---|---|
| | Products | of viable | of viable |
| Products | dilution | NCL-H1299 cells, % | T98G cells, % |
| Control | | 99.8 ± 4.2 | 97.2% |
| Ps name | | | |
| P_4C | 1:1 | 82.4 ± 8.8 | 58.7 ± 4.3 |
| | 1:10 | 101.8 ± 11.7 | 101.6 ± 5.0 |
| | 1:100 | 101.5 ± 11.4 | 99.1 ± 7.6 |
| P_4T | 1:1 | 14.5 ± 1.1* | 22.7 ± 4.8* |
| | 1:10 | 14.6 ± 2.6* | 24.2 ± 2.5* |
| | 1:100 | 42.5 ± 5.6* | 23.9 ± 4.1* |
| psP_4C | 1:1 | 14.4 ± 3.5 | 52.3 ± 2.3 |
| | 1:10 | 44.2 ± 6.2 | 100.6 ± 7.7 |
| | 1:100 | 99.6 ± 7.4 | 99.1 ± 9.2 |
| psP_4T | 1:1 | 14.2 ± 2.1 | 3.8 ± 2.7** |
| | 1:10 | 14.6 ± 2.7** | 12.1 ± 2.9** |
| | 1:100 | 15.1 ± 3.9** | 14.0 ± 3.4** |

*p < 0.05 compared to the relevant control;
p < 0.01

### Effect of Products Produced by Treated Cell for the Treatment of Infections

The provided data illustrated the effect of treated cells on sepsis by tracking body temperature variations in murine subjects following sepsis induction. These variations were measured at two distinct time intervals: 40 hours and 48 hours.

A mouse model of sepsis using fecal suspension intraperitoneal injection was used as described by Tsuchida et al (see, Tsuchida, T., Wada, T., Mizugaki, A., Oda, Y., Kayano, K., Yamakawa, K., & Tanaka, S. (2022). Protocol for a Sepsis Model Utilizing Fecal Suspension in Mice: Fecal Suspension Intraperitoneal Injection Model. Frontiers in Medicine, 9.).

In a disclosed system utilizing murine models' post-sepsis induction, bacteremia levels were quantitatively evaluated by measuring blood culture concentrations, presented in log₁₀ Colony Forming Units per milliliter (CFU/ml). The resultant data, recorded 20 hours post-sepsis induction, is articulated in the Table below:

TABLE 20

| | Blood culture [log10 | Median survival | |
|---|---|---|---|
| Mice group | CFU/ml] | (Time after sepsis induction, h) | |
| healthy | 0 | 0 | |
| saline | 7 ± 0 | 65 | h |
| P_4C | 6.1 ± 0.2 | 78 | h |
| P_4T | 3.3 ± 0.1 | >120 | h |

This quantification suggests an enhanced efficacy of the AMP_4T formulation in comparison to its AMP_4C counterpart and the saline control, with respect to the mitigation of bacteremia post-sepsis induction.

### Physical Properties of Products Produced by SL-4T Cells

Table Title: Assessment of the antibacterial activity of P following heat exposure for 30 minutes. (Table below)

Maximum dilution of the AMP sufficient to achieve MIC

| | P_4C | | | P_4T | | |
|---|---|---|---|---|---|---|
| Temperature | RT | 70° C. | 90° C. | RT | 70°C | 90°C |
| S. aureus VT-95 | ½ | 1/2 | ½ | ⅛ | ⅛ | ⅛ |
| B. pumilus VT 18 | ½ | 1/2 | na | ¼ | ¼ | ¼ |
| E. coli VT-57 | ½ | ½ | na | ⅛ | ⅛ | ⅛ |

RT Room temperature;
na not active

TABLE 21
Antibacterial activity of Products against bacteria after trypsin exposure
Maximum dilution of the Products sufficient to achieve MIC

| | Trypsin | | | | | | | | | | Proteinase K | | | | | | | | | |
|---|---|---|---|---|---|---|---|---|---|---|---|---|---|---|---|---|---|---|---|---|
| | P_4C | | | | | P_4T | | | | | P_4C | | | | | P_4T | | | | |
| Incubation | Con- | 30 | 2 | 15 | 30 | Con- | 30 | 2 | 15 | 30 | Con- | 30 | 2 | 15 | 30 | Con- | 30 | 2 | 15 | 30 |
| time | trol | sec | min | min | min | trol | sec | min | min | min | trol | sec | min | min | min | trol | sec | min | min | min |
| S. aureus | ½ | ½ | na | na | na | ⅛ | ⅛ | ¼ | ¼ | ½ | ½ | na | na | na | na | ⅛ | ⅛ | ½ | ½ | ½ |
| VT55 | | | | | | | | | | | | | | | | | | | | |

na not active

### Example 9. Effect of Different Activators on Treated Cells to Produce More Products

We treated SL4-T cells with various activators to generate biologically active products, using SL4-C products as a control. Both SL4-C and SL4-T, prepared as previously described with DNase, RNase, or antibody treatments, were subjected to activators, altering their secretions' properties. We evaluated the anticancer and antimicrobial effects of these products on various cancer cell lines and microbial isolates. Cancer cells were cultured in RPMI 1640 with 10% FBS to full confluency for 48-72 hours at 37° C. and 5% CO₂. MICs were determined using the broth microdilution method, following CLSI guidelines with modifications. Bacterial and fungal inoculums were standardized for testing, with outcomes detailed in

TABLE 22
Concentration of products produced by different cells
resulting in reducing viability of 50% cells.

| | | Antimicrobial activity |
|---|---|---|
| | Anticancer activity | A. niger/C. auris/ |
| | A549/NCL-H1299/ | S. aureus (VRSA)/ |
| | CT26/CW1474/ | B. cepacia/P. aeruginosa/ |
| Cells/Activator | SNU-5/PANC02 | A. baumannii |
| SL4-C/Alive Klebsiella pneumonia | n/a | n/a |
| SL4-C/Dead Klebsiella pneumonia | n/a | n/a |
| SL4-C/LPS | n/a | n/a |
| SL4-C/Carboxymethylcellulose pneumonia | n/a | n/a |
| SL4-C/Bacterial nucleic acids | n/a | n/a |
| SL4-C/NCL-H1299 (tumor cell line) | n/a | n/a |
| SL4-C/fibroblasts (non-tumor cell line) | n/a | n/a |
| SL4-C/T98G (tumor cell line) | 1:1 | 1:1 |
| SL4-C/Aspergillus spp | n/a | n/a |
| SL4-C/S. aureus | n/a | n/a |
| SL4-C/E. coli | n/a | n/a |
| SL4-C/P. aeruginosa | n/a | n/a |
| SL4-T/Alive Klebsiella pneumonia | >1:100* | >1:100* |
| SL4-T/Dead Klebsiella pneumonia | >1:100* | >1:100* |
| SL4-T/LPS | >1:100* | >1:100* |
| SL4-T/Carboxymethylcellulose pneumonia | >1:100* | >1:100* |
| SL4-T/Bacterial nucleic acids | >1:100* | >1:100* |
| SL4-T/NCL-H1299 (tumor cell line) | >1:100* | >1:100* |
| SL4-T/fibroblasts (non-tumor cell line) | >1:100* | >1:100* |
| SL4-T/T98G (tumor cell line) | >1:100* | >1:100* |
| SL4-T/Aspergillus spp | >1:100* | >1:100* |
| SL4-T/S. aureus | >1:100* | >1:100* |
| SL4-T/E. coli | >1:100* | >1:100* |
| SL4-T/P. aeruginosa | >1:100* | >1:100* |

n/a not active;
*p < 0.05

Data received indicate that immune cells following treatment and subsequent activation started to produce more potent products active against different pathogenic gram-positive, gram negative bacteria and fungi.

### Example 10. In Vitro Antimicrobial Susceptibility Testing

The minimum inhibitory concentrations (MICs) for the tested cells were determined using the broth microdilution method, following the Clinical and Laboratory Standards Institute (CLSI) guidelines with minor modifications. For bacterial testing, a standard bacterial inoculum of 5×10\hat{ }5 colony-forming units (CFU)/mL was employed.

In fungal testing, the standard inoculum size for yeast was set at 2.5×10\hat{ }3 CFU/mL, while for molds, it was 5×10\hat{ }4 CFU/mL. The minimum fungicidal concentration (MFC) was defined as the lowest concentration of the antimicrobial agent that completely inhibited visible fungal growth after 48 hours at 37° C. Microbial isolates were categorized as susceptible, intermediate, or resistant, based on susceptibility breakpoints for antifungals according to both CLSI and the European Committee on Antimicrobial Susceptibility Testing (EUCAST) criteria. Serial twofold dilutions of the antimicrobials were prepared in RPMI 1640 medium. The MIC was determined as the lowest concentration of cells that completely inhibited visible growth of the bacteria or fungi. All experiments were conducted in triplicate.

TABLE 23

| Probe | Name | Control | Treated |
|---|---|---|---|
| Granulocytes | GR | GR-C | GT-T |
| Agranulocytes | AGR | AGR-C | AGR-T |
| Granulocytes + platelets | SL1 | SL1-C | SL1-T |
| Agranulocytes + platelets | SL2 | SL2-C | SL2-T |
| Granulocytes + | SL21 | SL21-C | SL21-T |
| Agranulocytes + platelets | | | |
| White blood cells | SL3 | SL3-C | SL3-T |
| White blood cells + platelets | SL4 | SL4-C | SL4-T |

### Results

In vitro antimicrobial susceptibility testing of SL4 is presented in Table 24 below.

TABLE 24

| | Number | | | | |
|---|---|---|---|---|---|
| | of tested | Dilution of cells to reach MIC | | | |
| Tested strains | trains | SL4-C | SL4-T | SL21-C | SL21-T |
| S. aureus MRSA/VRSA | 20 | 1.0-0.1 | 0.0001* | 1.0-0.1 | 0.0001* |
| Bacillus pumilus | 10 | 1:1 | 0.0001-0.00001* | 1:1 | 0.0001* |
| Enterococcus faecalis | 20 | 1:1 | 0.00001* | 1:1 | 0.00001* |
| Pseudomonas | 20 | >1:1 | 0.01-0.001* | >1:1 | 0.01* |
| aeruginosa | | | | | |
| Burcholderia | 10 | 1.0-0.1 | 0.00001* | 1.0-0.1 | 0.00001* |
| cenocepacia | | | | | |
| Acinetobacter baumannii | 20 | 1.0-0.1 | 0.00001* | 1.0-0.1 | 0.00001* |
| Escherichia coli | 20 | >1:1 | 0.001-0.0001* | >1:1 | 0.001* |
| Serratia marcescens | 10 | 1.0 | 0.0001* | 1.0 | 0.0001* |
| Citrobacter freundii | 10 | 1.0-0.1 | 0.0001* | 1.0-0.1 | 0.0001* |
| Aeromonas caviae | 10 | 1.0-0.1 | 0.01-0.0001* | 1.0-0.1 | 0.0001* |
| Treponema palidum | 10 | 1.0 | 0.00001* | 1.0 | 0.0001* |
| Neisseria gonorrhoeae | 10 | 1.0-0.1 | 0.01-0.0001* | 1.0-0.1 | 0.001* |
| Treponema denticola | 10 | 1.0 | 0.00001* | 1.0 | 0.001* |
| Prevotella | 10 | 1.0-0.1 | 0.00001* | 1.0-0.1 | 0.00001* |
| melaninogenica | | | | | |
| F. nucleatum | 10 | 1.0-0.1 | 0.0001* | 1.0-0.1 | 0.0001* |
| Klebsiella pneumoniae | 20 | 1.0-0.1 | 0.00001* | 1.0-0.1 | 0.0001* |
| Candida glabrata | 20 | >1:1 | 0.1-0.01* | >1:1 | 0.1-0.01* |
| Candida tropicalis | 20 | 1.0 | 0.00001* | 1.0 | 0.0001* |
| Candida albicans | 20 | 1.0 | 0.00001* | 1.0 | 0.00001* |
| Aspergillus fumigatus | 20 | 1:1 | 0.001-0.0001* | 1:1 | 0.001* |
| Aspergillus niger | 20 | >1:1 | 0.001-0.0001* | >1:1 | 0.0001* |
| Cryptococcus | 10 | 1.0-0.1 | 0.001-0.0001* | 1.0-0.1 | 0.0001* |
| neoformans | | | | | |
| Mycobacterium | 10 | >1:1 | 0.1-0.01* | >1:1 | 0.1-0.01* |
| smegmatis | | | | | |

*p < 0.05

In vitro antimicrobial susceptibility testing of different tested products is presented in the Table below.

TABLE 25

| | | | | AGR- | | | | | | AGR- | | |
|---|---|---|---|---|---|---|---|---|---|---|---|---|
| | SL4-C | AGR-C | GR-C | C+GR-C | SL2-C | SL1-C | SL4-T | AGR-T | GR-T | T+GR-T | SL2-T | SL1-T |
| S. aureus | 0.1 | >1 | >1 | 1 | 1 | 1:10 | 0.00001 | 0.1 | 0.001 | 0.00001 | 0.001 | 0.00001 |
| MRSA VT | | | | | | | | | | | | |
| 4490 | | | | | | | | | | | | |
| P. aeruginosa | >1 | >1 | >1 | 1 | 1 | 1 | 0.01 | 1 | 1 | 0.01 | 1 | 0.01 |
| MR45 | | | | | | | | | | | | |
| K. pneumoniae | >1 | >1 | >1 | >1 | >1 | >1 | 0.1 | 1 | 1 | 0.1 | 1 | 0.1 |
| VT 1698 | | | | | | | | | | | | |
| A. niger | >1 | >1 | >1 | >1 | >1 | >1 | 0.001 | 0.01 | 1 | 0.001 | 1 | 0.001 |
| VT 1591 | | | | | | | | | | | | |

The data received indicate that treatment enhanced the cells antimicrobial activity against a wide range of Gram-positive and Gram-negative bacteria, both aerobic and anaerobic, as well as against various fungi.

### Example 11. Antibiofilm Activity of Treated Cells

Treated cells efficacy was evaluated in a biofilm model using a 96-well plate with 200 μL of 5×10\hat{ }5 CFU/mL bacterial suspension in LB broth, incubated at 37° C. for 24 hours to form biofilms. After incubation, biofilms were washed with PBS, exposed to RPMI 1640 with tested cells for 24 hours, and then assessed by OD600 measurements, with all procedures done in triplicate.

White blood cells were modified with DNase and RNase or with specific anti-DNA and anti-RNA antibodies, produced by immunizing rabbits with nucleic acids from different gram positive and negative bacteria and Freund's adjuvant. Additionally, human bone marrow CD34+ stem cells and fibroblasts, the latter isolated from juvenile foreskin and cultured through several passages, were also treated with DNase and RNase treatments for comparison. The results of biofilm's biomass quantification after the treatment with different cells are presented in Table 26 For P. aeruginosa biofilms we used treated cells the low dose of 10*3 cells/mL.

TABLE 26
The effect of cells on the inhibition of the biofilm biomass.

| | Biofilm biomass inhibition [%] | | |
|---|---|---|---|
| | S. aureus | C. albicans | P. aeruginosa |
| Probe | VT-209 | MRS-8 | MR-45 |
| Granulocytes (untreated) | 4.6 ± 3.9 | 2.2 ± 1.4 | — |
| Agranulocytes (untreated) | 5.2 ± 2.7 | 1.6 ± 2.0 | — |
| Granulocytes + platelets (untreated) | 4.1 ± 3.2 | 3.9 ± 2.1 | — |
| Agranulocytes + platelets (untreated) | 4.6 ± 3.0 | 5.0 ± 2.6 | — |
| Granulocytes + Agranulocytes + platelets | 2.2 ± 1.8 | 2.5 ± 2.2 | — |
| (untreated) | | | |
| White blood cells (untreated) | 4.8 ± 2.7 | 4.3 ± 0.8 | — |
| White blood cells + platelets (untreated) | 6.2 ± 2.1 | 3.7 ± 1.2 | — |
| Granulocytes (T) | 64.4 ± 9.4* | 71.5 ± 8.5* | — |
| Agranulocytes (T) | 69.5 ± 6.1* | 67.3 ± 7.2* | — |
| Granulocytes + platelets (T) | 86.0 ± 9.5* | 79.7 ± 5.6* | — |
| Agranulocytes + platelets (treated with | 87.2 ± 10.1* | 83.2 ± 9.3* | — |
| nucleases) | | | — |
| Granulocytes + Agranulocytes + platelets | 93.6 ± 5.1* | 92.7 ± 6.3* | — |
| (T) | | | |
| White blood cells(T) | 42.5 ± 6.3* | 42.7 ± 6.9* | 45.4 ± 7.1* |
| White blood cells + platelets (T) | 94.2 ± 5.6* | 93.9 ± 5.2* | — |
| Granulocytes (Ta) | 75.3 ± 8.0* | 74.6 ± 9.5* | — |
| Agranulocytes (Ta) | 81.7 ± 5.2* | 78.4 ± 8.8* | — |
| Granulocytes + platelets (Ta) | 82.9 ± 7.3* | 85.2 ± 9.6* | — |
| Agranulocytes + platelets (Ta) | 85.5 ± 7.8* | 82.9 ± 6.9* | — |
| Granulocytes + Agranulocytes + platelets | 91.4 ± 5.7* | 92.0 ± 7.2* | — |
| (Ta) | | | |
| White blood cells(Ta) | 72.3 ± 6.3* | 71.2 ± 7.4* | — |
| White blood cells + platelets (Ta) | 91.9 ± 8.1* | 91.5 ± 8.0* | — |
| White blood cells + platelets + human bone | — | — | 68.3 ± 9.5* |
| marrow CD34 + stem cells (T) | | | |
| White blood cells + platelets + fibroblasts | — | — | 52.2 ± 10.8* |
| (T) | | | |

*p < 0.05;
C—untreated;
T—treated with nucleases,
Ta—treated with anti-DNA/RNA antibodies

### Example 12

The activity of treated immune cells against persisters was analyzed. In order to obtain persisters, E. coli ATCC 25922 was inoculated in LB broth supplemented with ampicillin (150 μg/mL), and incubated at 37° C. for 24 h. After that cells were collected and treated with SL4-C or SL-4T for 10 minutes. Subsequently, bacteria were inoculated again on LB agar and the number of viable counts was evaluated.

TABLE 27
Effect of treated cells on persisters.

| Untreated control | SL4-C | SL4-T |
|---|---|---|
| 0.000767 ± 0.000233 | 0.00062 ± 0.000432 | 0.00000133 ± 0.00000047* |

*p < 0.05

### Example 13. Antimicrobial Activity Using Chicken Embryo Sepsis Model and Evaluation of the Safety of Allogeneic Treated Cells

Bacteria Preparation: We used Staphylococcus aureus VT-232, Klebsiella pneumonia VT-678, and Pseudomonas aeruginosa VT-548 strains. The bacterial cells were cultured on 5% blood agar, then in Columbia broth to OD 0.50 at 540 nm, centrifuged, and resuspended in 0.9% NaCl.

Chicken Embryo Inoculation: Used pathogen-free eggs, incubated at 37° C. and 65% humidity.

At 12 days, embryos were intravenously inoculated with 0.1 ml bacterial suspension. Viability checked by candling post 5 hours and daily for 4 days.

Injection of Testing Cells: Administered 0.1 mL of SL4-C, SL4-T, or saline (control) 3 hours after bacterial inoculation.

Bacterial Strain Distribution: Collected and homogenized samples from blood, liver, and brain to determine CFU/ml.

Results: Control group showed 0% mortality, confirming non-toxicity of agents.

SL4-T treatment significantly reduced mortality in embryos infected with SA, KP, and PA strains, demonstrating its protective effectiveness against bacterial infections.

TABLE 28
The effect of treated cells on the protection from
septic related mortality.

| | Mi- | Agent for | Mortality, % | | | |
|---|---|---|---|---|---|---|
| Groups | crobe | treatment | after 24 h | after 48 h | after 72 h | after 96 h |
| Control | Saline | Saline | 0% | 0% | 0% | 0% |
| SL4-C | Saline | SL4-C | 0% | 0% | 0% | 0% |
| SL4-T | Saline | SL4-T | 0% | 0% | 0% | 0% |
| SA #1 | SA | Saline | 73.7% | 84.2% | 94.7% | 100% |
| SA #2 | SA | SL4-C | 55.5% | 83.3% | 88.9% | 100% |
| SA #3 | SA | SL4-T | 10.5%* | 15.8%* | 26.3%* | 26.3%* |
| KP #1 | KP | Saline | 73.7% | 78.9% | 100% | 100% |
| KP #2 | KP | SL4-C | 65% | 65% | 80% | 95% |
| KP #3 | KF | SL4-T | 10.5%* | 26.3%* | 31.6%* | 31.6%* |
| PA #1 | PA | Saline | 84.2% | 100% | 100% | 100% |
| PA #2 | PA | SL4-C | 65% | 85% | 95% | 95% |
| PA #3 | PA | SL4-T | 5.2%* | 10.5%* | 10.5%* | 26.3%* |

*p < 0.05

### Example 14. Effect of Treated Cells on Mouse Sepsis Model

### Sepsis Model Utilizing Fecal Suspension

A total of 8,000 mg of mouse feces were collected and resuspended in 55 mL of 0.9% saline solution. During the preparation phase, the feces were manually macerated using a tea strainer until thoroughly soaked in the saline. The macerated feces were then filtered through the same strainer with the aid of a grinding rod, processing the material until it reached a non-gritty consistency. For further refinement, the fecal suspension was passed through a 70 μm filter. The suspension was thoroughly mixed, and 1 mL was intraperitoneally injected using a 25-gauge needle into the mouse's intraperitoneal cavity.

The mice were treated with saline, SL4-C, or SL4-T 6 hours after the induction of sepsis.

Bacterial Load 40h and 46h after Sepsis Induction

The number of colony-forming units (CFU) was evaluated by plating the blood on LB petri dishes (Table 29).

TABLE 29
Blood culture [log10 CFU/ml].

| | Time after sepsis induction, h |
|---|---|
| Mice group | 20 |
| Healthy | 0 |
| Saline | 4.7 ± 0.2* |
| SL4-C | 4.2 ± 0.2* |
| SL4-T | 1.3 ± 0.3 |

*p < 0.05 compared with SL4-T

Next, the median survival time of different mice groups post-sepsis induction were analyzed (Table 3).

TABLE 30
Median survival.

| Mice group | Time after sepsis induction, h | |
|---|---|---|
| Healthy | 0 | |
| Saline | 65 | h* |
| SL4-C | 78 | h* |
| SL4-T | >120 | h |

*p < 0.05 compared with SL4-T

### Example 15. The Effect of Treated Cells on Enveloped and Non-Enveloped Viruses

The treated SL4-T were obtained as previously discussed following the repeated treatment with nucleases or ant-DNA or anti-RNA antibodies.

### In Vitro Log Reduction Virucidal Assay

The cell cultures were grown in tissue culture media (Eagle's Minimum Essential Medium (MEM) supplemented with 10% fetal bovine serum (all Sigma, MO). For the log reduction virucidal assay from 106 plaque-forming units per milliliter (PFU/mL) of viruses were used. 0.1 ml of each testing cells were added to 0.9 ml of each of viruses tested, thus the final concentrations were diluted in 10 times. The viral mixtures were incubated at 37 C for 2h, and next 0.01 ml aliquots were taken and dissolved in 990 ml of the appropriate tissue culture. Next, the viral titers were Log 10 converted and for each tray Log 10 reductions compared to negative control (vehicle) were calculated. The results were plotted as Log 10 at each time bin. The reduction in viral titer of 3 Log 10 (99.9%) were considered to be virucidal.

Data received showed antiviral activity of treated cells against enveloped and non-enveloped viruses.

### Example 16. Efficacy of Inhaled Treated Immune Cells for the Treatment of Respiratory Infections

To study the efficacy of treated cells, a single inhaled drug was evaluated in mouse model of pneumonia. To develop pneumonia, adult C57BL/6 mice were used. Animals were randomized into three groups of eight, which were used to measure overall survival following the treatment with saline, SL4-C or SL4-T. Mice were then anesthetized with 2% isoflurane, and nasally instilled with P. aeruginosa suspension. Briefly, nares were blocked, and mice aspirated 50 μL P. aeruginosa VT57 into the lungs while being held vertically for 60 s. Mice received a total dose of 8.5 log 10 cFU/mouse. Overall survival was assessed over 5 days.

After 8h post infection, mice were placed in a chamber of 40 dm³ and using a connected PARI BABY® N compressor and PARI LL Nebuliser saline, SL4-C or SL4-T were dispersed in the camera. The inhalation dose was adjusted to the length of time the animals were being placed in the experimental chamber. The period of time during which the animals were exposed to the inhalation was determined by the formula:

$$Length of inhalation = Dose ( cells / kg ) / A \times B ,$$

A—the maximum possible concentration of the tested compound in the aerosol (in the chamber), was based on the technical characteristics of the nebulizer, determining the rate of aerosol formation;
B—a minute ventilation of mice-2.67 L/kg body weight of animals;
Time of inhalation of a therapeutic dose was 8.3 min.

The results are shown in table 31.

TABLE 31
Median survival following treatment with different tested therapies.

| | Time after lung infection (pneumonia) | |
|---|---|---|
| Mice group | induction, hours (h) | |
| Saline | 18 | h* |
| SL4-C | 22 | h* |
| SL4-T | >120 | h |

*p < 0.05 compared with SL4-T

### Example 17. The Use of Treated Cells for the Treatment of Chronic Infections

The research aimed to assess treated cells' effectiveness in treating delayed wound healing in diabetic ulcers with polymicrobial biofilms in a mouse model. Using clinical isolates S. aureus MRSA VTR71, P. aeruginosa VR-465, and E. faecalis VT-23, bacteria were cultured on Mueller-Hinton agar, followed by broth subcultures. MICs were determined by CLSI standards. The study involved 16 diabetic female mice (db/db; BKS.Cg-Dock7m+/+Leprdb/J), 12 weeks old, anesthetized and subjected to a full-thickness wound. A mixed biofilm inoculum (8.15 log 10 CFU/ml) was applied to each wound after 3 days of initial bandaging.

Group 1: White blood cells+platelets (untreated); Group 2: White blood cells+platelets+fibroblasts (untreated); Group 3: White blood cells+platelets (treated); Group 4: White blood cells+platelets+fibroblasts (treated).

The percentage of wound closure was determined using the equation: (Ad0−Adx)/Ad0×100%, where Ad0 is the wound area on day 0 and Adx is the area of the wound on the observation day.

TABLE 32
Effect of tested cells on polymicrobial biofilm dynamics within chronic wounds.

| | | | | | Wound closure |
|---|---|---|---|---|---|
| | | | | | on day 10 |
| | Day of | | | | (% of original |
| Group | therapy | P. aeruginosa | S. aureus | E. faecalis | wound size) |
| 1 | 0 | 7.5log10 ± 1.3 | 7.1log10 ± 1.3 | 7.2log10 ± 1.7 | |
| | 10 | 6.8log10 ± 1.2 | 7.7log10 ± 0.7 | 8.2log10 ± 0.8 | 104 ± 8 |
| 2 | 0 | 7.5log10 ± 1.3 | 7.1log10 ± 1.3 | 7.2log10 ± 1.7 | |
| | 10 | 7.1log10 ± 0.8 | 7.6log10 ± 1.2 | 7.9log10 ± 1.1 | 102 ± 18 |
| 3 | 0 | 7.5log10 ± 1.3 | 7.1log10 ± 1.3 | 7.2log10 ± 1.7 | |
| | 10 | 1.4log10 ± 0.2* | 0* | 0* | 38 ± 6* |
| 4 | 0 | 7.5log10 ± 1.3 | 7.1log10 ± 1.3 | 7.2log10 ± 1.7 | |
| | 10 | 0* | 0* | 0* | 11 ± 5* |

*p < 0.05

### Example 18. The Impact of Immune Cell Treatment on the Transcriptome Activity in Antimicrobial Pathways of Immune Cells

SL4-C and SL4-T were obtained as previously described. To isolate RNA from cells, the cell suspension were washed thrice in PBS, pH 7.2 (Sigma) and centrifuged each time at 4000×g for 20 min (Microfuge 20R, Beckman Coulter) followed by resuspension in PBS.

RNA was purified using the RNeasy Mini Kit (Qiagen), according to the manufacturer's protocol. The concentration and quality of RNA based on absorbance at 230, 260, and 280 nm was determined with the NanoDrop OneC spectrophotometer (Thermo Fisher Scientific).

Transcriptome sequencing (RNA-Seq) libraries were prepared using an Illumina TruSeq Stranded Total RNA Library Prep kit. RNA was ribo-depleted with the Epicenter Ribo-Zero magnetic gold kit (catalog No. RZE1224), according to the manufacturer's recommendations. The libraries were pooled equimolarly and sequenced in an Illumina NextSeq 500 (Illumona, San Diego, CA, USA) platform with paired 150-nucleotide reads (130 MM reads max).

Sequencing reads were mapped corresponding to the reference genome of human immune cells, and expression levels were estimated using Geneious 11.1.5. Transcripts with an adjusted p value of <0.05 and log 2 fold change value of +0.5 were considered for significant differential expression. PCA, volcano plots were generated using the ggplot2 package in R, and the Venn diagram was obtained using BioVenn. Differentially expressed genes (DEGs) were identified as genes with a two-fold change (log 2 fold-change >0.5 or <−0.5) and false discovery rate <0.05. The results are set forth in the Table below.

TABLE 33
DEGs within selected pathways

| Immune cell signaling | Immune cell migration | Cell membrane transport |
|---|---|---|
| BCR signaling | Focal adhesion pathway | Phagocytosis |
| Chemokine signaling | Leukocyte transedothelial | Endocytosis |
| | migration | |
| Cytokine-cytokine receptor interaction | Regulation of actin | |
| | cytoskeleton | |
| MAPK signaling pathway | Membrane invagination | |
| PPAR signaling pathway | | |
| TGFb signaling | | |
| Hematopoietic cell lineage | | |
| Integrin-mediated signaling pathway | | |

### Example 19. Effect of Treated Cells on Chronic Inflammatory Diseases

We investigated the effectiveness of treated cells in a model of interstitial cystitis, a disease characterized by mast cell abnormalities and alterations in the p38/NF-kB pathway. A chemically induced rat model of interstitial cystitis was used. Adult female Wistar rats (180-230 g) were anesthetized with isoflurane. Their bladders were catheterized, the urine was removed, and 200 μl of 0.1 N HCl was instilled for 4 minutes. This process was repeated after one week. Forty-eight hours following the second HCl instillation, rats under isoflurane anesthesia received intravesical instillations of either SL4-C or SL4-T (10\hat{ }6 cells in 50 μl of PBS) or SL4-C or SL4-T (10\hat{ }6 cells in 10 μl of PBS), injected into both the posterior and anterior bladder walls. This procedure was repeated every 72 hours, three times in total. The levels of TNF-a and histamine in urine were analyzed using the ELISA method 7 and 14 days after the last therapy with SL4-C/SL4-T (Table 34).

TABLE 34
Severity of Interstitial Cystitis.

| | | SL4-C | SL4-C | SL4-T | SL4-T |
|---|---|---|---|---|---|
| Control | Sham | (instilled) | (injected) | (instilled) | (injected) |
| 100 ± 0 | 1040 ± 107 | 940 ± 135 | 860 ± 116 | 180 ± 93* | 150 ± 54* |

*p < 0.05 compared to SL4-C

### Example 20. The Effect of Treated Cells on Intracellular Parasites

We studied the effect of SL4-C and SL4-T on the malaria-related protozoan Plasmodium falciparum. Both SL4-C and SL4-T were obtained as described previously.

P. falciparum was cultured in human type O blood and ATCC Medium 2196, following the manufacturer's instructions, with the goal of maintaining parasitemia at 3-5% in the red blood cell culture. The samples were incubated with various dilutions of SL4-C and SL4-T for 72 hours. Subsequently, the quantity of histidine-rich protein 2 (HRP2) produced by P. falciparum was measured using an HRP2 ELISA, according to the manufacturer's guidelines. The individual inhibitory concentrations 50% (IC50) and 90% (IC90) were calculated. The results are presented in Table 35.

TABLE 35
Antiparasitic Activity Against Malaria of SL4-C
and SL4-T Depending on Their Dilution.

| Probe | IC50 | IC90 |
|---|---|---|
| SL4-C | >1 | >1 |
| SL4-T | 0.001-0.0001* | 0.01-0.001* |

*p < 0.05

### Example 21. The Use of Treated Cells to Increase Productivity of Mammalian Cell Lines

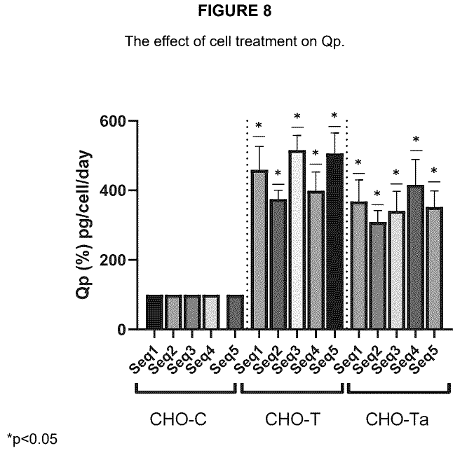

We utilized Chinese hamster ovary (CHO) cells expressing monoclonal antibodies Seq 1-5, with treatments as follows: control (CHO-C), DNase and RNase-treated (CHO-T), and anti-DNA/RNA antibody-treated (CHO-Ta). Maintenance was in shake flasks before scaling to 5 L bioreactors (Eppendorf), with a daily vessel volume exchange. See FIG. 8 for the impact of treatment on Qp.

We assessed CHO-T cell response to reduced nutrient medium volumes, examining viability and growth when daily exchange decreased from 1 vessel volume to 0.5 or 0.25. Analysis was conducted on day 10 (Table 1).

TABLE 35
Viability of cells depending on volume of media used.

Viability (%)

| Vessel volume | CHO-C | CHO-T |
|---|---|---|
| 0.5 | 51 | 95* |
| 0.25 | 29 | 94* |

*p < 0.05

SEQ ID NO. 1
Siltuximab
(Heavy chain)
EVQLVESGGK LLKPGGSLKL SCAASGFTFS SFAMSWFRQS PEKRLEWVAE ISSGGSYTYY
PDTVTGRFTI SRDNAKNTLY LEMSSLRSED TAMYYCARGL WGYYALDYWG QGTSVTVSSA
STKGPSVFPL APSSKSTSGG TAALGCLVKD YFPEPVTVSW NSGALTSGVH TFPAVLQSSG
LYSLSSVVTV PSSSLGTQTY ICNVNHKPSN TKVDKKVEPK SCDKTHTCPP CPAPELLGGP
SVFLFPPKPK DTLMISRTPE VTCVVVDVSH EDPEVKFNWY VDGVEVHNAK TKPREEQYNS
TYRVVSVLTV LHQDWLNGKE YKCKVSNKAL PAPIEKTISK AKGQPREPQV YTLPPSRDEL
TKNQVSLTCL VKGFYPSDIA VEWESNGQPE NNYKTTPPVL DSDGSFFLYS KLTVDKSRWQ
QGNVFSCSVM HEALHNHYTQ KSLSLSPGK
(Light chain)
QIVLIQSPAI MSASPGEKVT MTCSASSSVS YMYWYQQKPG SSPRLLIYDT SNLASGVPVR
FSGSGSGTSY SLTISRMEAE DAATYYCQQW SGYPYTFGGG TKLEIKRTVA APSVFIFPPS
DEQLKSGTAS VVCLLNNFYP REAKVQWKVD NALQSGNSQE SVTEQDSKDS TYSLSSTLTL
SKADYEKHKV YACEVTHQGL SSPVTKSFNR GEC
(Disulfide bridge: H22-H96, H146-H202, H263-H323, H369-H427, H228-H′228,
H231-H′231, L23-L87, L133-L193, H222-L213)
SEQ ID NO. 2
Atezolizumab
(Heavy chain)
EVQLVESGGG LVQPGGSLRL SCAASGFTFS DSWIHWVRQA PGKGLEWVAW ISPYGGSTYY
ADSVKGRFTI SADTSKNTAY LQMNSLRAED TAVYYCARRH WPGGFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKKVEPKS CDKTHTCPPC PAPELLGGPS
VFLFPPKPKD TLMISRTPEV TCVVVDVSHE DPEVKFNWYV DGVEVHNAKT KPREEQYAST
YRVVSVLTVL HQDWLNGKEY KCKVSNKALP APIEKTISKA KGQPREPQVY TLPPSREEMT
KNQVSLTCLV KGFYPSDIAV EWESNGQPEN NYKTTPPVLD SDGSFFLYSK LTVDKSRWQQ
GNVFSCSVMH EALHNHYTQK SLSLSPGK
(Light chain)
DIQMTQSPSS LSASVGDRVT ITCRASQDVS TAVAWYQQKP GKAPKLLIYS ASFLYSGVPS
RFSGSGSGTD FTLTISSLQP EDFATYYCQQ YLYHPATFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
(Disulfide bridge: H22-H96, H145-H201, H221-L214, H227-H′227, H230-H′230,
H262-H322, H368-H426, H′22-H′96, H′145-H′201, H′221-L′214, H′262-H′322,
H′368-H′426, L23-L88, L134-L194, L′23-L′88, L′138-L′194)
SEQ ID NO. 3
Erenumab [symbol]
(Heavy chain)
QVQLVESGGG VVQPGRSLRL SCAASGFTFS SFGMHWVRQA PGKGLEWVAV ISFDGSIKYS
VDSVKGRFTI SRDNSKNTLF LQMNSLRAED TAVYYCARDR LNYYDSSGYY HYKYYGMAVW
GQGTTVTVSS ASTKGPSVFP LAPCSRSTSE STAALGCLVK DYFPEPVTVS WNSGALTSGV
HTFPAVLQSS GLYSLSSVVT VPSSNFGTQT YTCNVDHKPS NTKVDKTVER KCCVECPPCP
APPVAGPSVF LFPPKPKDTL MISRTPEVTC VVVDVSHEDP EVQFNWYVDG VEVHNAKTKP
REEQFNSTFR VVSVLTVVHQ DWLNGKEYKC KVSNKGLPAP IEKTISKTKG QPREPQVYTL
PPSREEMTKN QVSLTCLVKG FYPSDIAVEW ESNGQPENNY KTTPPMLDSD GSFFLYSKLT
VDKSRWQQGN VFSCSVMHEA LHNHYTQKSL SLSPGK
(Light chain)
QSVLTQPPSV SAAPGQKVTI SCSGSSSNIG NNYVSWYQQL PGTAPKLLIY DNNKRPSGIP
DRFSGSKSGT STTLGITGLQ TGDEADYYCG TWDSRLSAVV FGGGTKLTVL GQPKANPTVT
LFPPSSEELQ ANKATLVCLI SDFYPGAVTV AWKADGSPVK AGVETTKPSK QSNNKYAASS
YLSLTPEQWK SHRSYSCQVT HEGSTVEKTV APTECS
(Disulfide bridge: H22-H96, H144-L215, H157-H213, H232-H′232, H233-H′233,
H236-H′236, H239-H′239, H270-H330, H376-H434, H′22-H′96, H′144-L′215,
H′157-H′213, H′270-H′330, H′376-H′434, L22-L89, L138-L197, L′22-L′89,
L′138-L′197)
SEQ ID NO. 4
Cemiplimab
(Heavy chain)
EVQLLESGGV LVQPGGSLRL SCAASGFTFS NFGMTWVRQA PGKGLEWVSG ISGGGRDTYF
ADSVKGRFTI SRDNSKNTLY LQMNSLKGED TAVYYCVKWG NIYFDYWGQG TLVTVSSAST
KGPSVFPLAP CSRSTSESTA ALGCLVKDYF PEPVTVSWNS GALTSGVHTF PAVLQSSGLY
SLSSVVTVPS SSLGTKTYTC NVDHKPSNTK VDKRVESKYG PPCPPCPAPE FLGGPSVFLF
PPKPKDTLMI SRTPEVTCVV VDVSQEDPEV QFNWYVDGVE VHNAKTKPRE EQFNSTYRVV
SVLTVLHQDW LNGKEYKCKV SNKGLPSSIE KTISKAKGQP REPQVYTLPP SQEEMTKNQV
SLTCLVKGFY PSDIAVEWES NGQPENNYKT TPPVLDSDGS FFLYSRLTVD KSRWQEGNVF
SCSVMHEALH NHYTQKSLSL SLGK
(Light chain)
DIQMTQSPSS LSASVGDSIT ITCRASLSIN TFLNWYQQKP GKAPNLLIYA ASSLHGGVPS
RFSGSGSGTD FTLTIRTLQP EDFATYYCQQ SSNTPFTFGP GTVVDFRRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
(Disulfide bridge: H22-H96, H131-L214, H144-H200, H223-H′223, H226-H′226,
H258-H318, H364-H422, H′22-H′96, H′131-L′214, H′114-H′200, H′258-H′318,
H′364-H′422, L23-L88, L134-L194, L′23-L′88, L′134-L′194)
SEQ ID NO. 5
Isatuximab
(A chain)
QVQLVQSGAE VAKPGTSVKL SCKASGYTFT DYWMQWVKQR PGQGLEWIGT IYPGDGDTGY
AQKFQGKATL TADKSSKTVY MHLSSLASED SAVYYCARGD YYGSNSLDYW GQGTSVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKFNW YVDGVEVHNA KTKPREEQYN
STYRVVSVLT VLHQDWLNGK EYKCKVSNKA LPAPIEKTIS KAKGQPREPQ VYTLPPSRDE
LTKNQVSLTC LVKGFYPSDI AVEWESNGQP ENNYKTTPPV LDSDGSFFLY SKLTVDKSRW
QQGNVFSCSV MHEALHNHYT QKSLSLSPGK
(B chain)
QVQLVQSGAE VAKPGTSVKL SCKASGYTFT DYWMQWVKQR PGQGLEWIGT IYPGDGDTGY
AQKFQGKATL TADKSSKTVY MHLSSLASED SAVYYCARGD YYGSNSLDYW GQGTSVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKFNW YVDGVEVHNA KTKPREEQYN
STYRVVSVLT VLHQDWLNGK EYKCKVSNKA LPAPIEKTIS KAKGQPREPQ VYTLPPSRDE
LTKNQVSLTC LVKGFYPSDI AVEWESNGQP ENNYKTTPPV LDSDGSFFLY SKLTVDKSRW
QQGNVFSCSV MHEALHNHYT QKSLSLSPGK
(C chain)
DIVMTQSHLS MSTSLGDPVS ITCKASQDVS TVVAWYQQKP GQSPRRLIYS ASYRYIGVPD
RFTGSGAGTD FTFTISSVQA EDLAVYYCQQ HYSPPYTFGG GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
(D chain)
DIVMTQSHLS MSTSLGDPVS ITCKASQDVS TVVAWYQQKP GQSPRRLIYS ASYRYIGVPD
RFTGSGAGTD FTFTISSVQA EDLAVYYCQQ HYSPPYTFGG GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
(Disulfide bridge: A22-A96, A147-A203, A223-C214, A229-B229, A232-B232,
A264-A324, A370-A428, B22-B96, B147-B203, B223-D214, B264-B324, B370-B428,
C23-C88, C134-C194, D23-D88, D134-D194)

### Example 22. The Use of Treatment Cells for the AAV Production and Cell Expansion Systems

HEK 293T cells from ATCC were treated with DNase and RNase at varying concentrations for 1 min to 24h, resulting in: 293T-T1 (DNase/RNase treated), 293T-T2 (DNase/RNase cultured), and 293T-Tr (RNase cultured). Control cells (293T-C) received PBS instead. Upon reaching 70% confluency, cells in serum-free medium were transfected with pHLP-AAV5 (AAP5+) and pCMV1-AAV5cmVP3 using polyethyleneimine (DNA/polyethyleneimine ratio of 1:2) and harvested at 120 hours for viral particle recovery. The pHLP-AAV5 plasmid, a standard helper for AAV5 vector production, expresses AAV2 Rep and AAV5 proteins. Cells and medium were purified 120 hours post-transfection via cesium chloride and density gradient centrifugation, with results in Table 36 below.

TABLE 36
AAV yield (%) ± SD

| 293-C | 293-T1 | 293-T2 | 293-Tr |
|---|---|---|---|
| 100 ± 0 | 496 ± 15.6* | 391 ± 52.7* | 228 ± 47.7* |

*p < 0.05

### Example 23. Treatment of Yeast Expression System for the Production of Recombinant Proteins

Insulin producing Komagataella phaffii (Pichia pastoris) was obtained by transformation of Komagataella phaffii (Pichia pastoris) SuperMan5 strain with yeast vector pPICZ-α cloned with insulin DNA (Sequence 6). Fungi from a single colony underwent treatment with cycles of DNase and RNase treatment (—T), RNase presence (—Tr), or PBS as a control (—C). Inoculated with 25 mL MGYH in a 250 mL flask, they were grown at +30° C. and 300 rpm until OD600=4. After centrifuging at 1,500 g for 10 min, the cell pellet was resuspended in 100 mL MMH medium to OD600 of 0.9-1.0, transferred to a 1-liter flask, and induced with methanol. Recombinant protein levels in the supernatant are set forth in the Table below. The use of treatment cells to increase production of hormones.

TABLE 37

| | Insulin (%) | |
|---|---|---|
| K. phaffii-C | K. phaffii-T | K. phaffii-Tr |
| 100 | 457 ± 28* | 244 ± 30* |

*p < 0.05

Data received indicate that the use of treatment cells increased the production of the recombinant proteins.

SEQ ID NO. 6
AGCCCTCCAGGACAGGCTGCATCAGAAGAGGCCATCAAGCAGGTCTGTT
CCAAGGGCCTTTGCGTCAGGTGGGCTCAGGATTCCAGGGTGGCTGGACC
CCAGGCCCCAGCTCTGCAGCAGGGAGGACGTGGCTGGGCTCGTGAAGCA
TGTGGGGGTGAGCCCAGGGGCCCCAAGGCAGGGCACCTGGCCTTCAGCC
TGCCTCAGCCCTGCCTGTCTCCCAGATCACTGTCCTTCTGCCATGGCCC
TGTGGATGCGCCTCCTGCCCCTGCTGGCGCTGCTGGCCCTCTGGGGACC
TGACCCAGCCGCAGCCTTTGTGAACCAACACCTGTGCGGCTCACACCTG
GTGGAAGCTCTCTACCTAGTGTGCGGGGAACGAGGCTTCTTCTACACAC
CCAAGACCCGCCGGGAGGCAGAGGACCTGCAGGGTGAGCCAACTGCCCA
TTGCTGCCCCTGGCCGCCCCCAGCCACCCCCTGCTCCTGGCGCTCCCAC
CCAGCATGGGCAGAAGGGGGCAGGAGGCTGCCACCCAGCAGGGGGTCAG
GTGCACTTTTTTAAAAAGAAGTTCTCTTGGTCACGTCCTAAAAGTGACC
AGCTCCCTGTGGCCCAGTCAGAATCTCAGCCTGAGGACGGTGTTGGCTT
CGGCAGCCCCGAGATACATCAGAGGGTGGGCACGCTCCTCCCTCCACTC
GCCCCTCAAACAAATGCCCCGCAGCCCATTTCTCCACCCTCATTTGATG
ACCGCAGATTCAAGTGTTTTGTTAAGTAAAGTCCTGGGTGACCTGGGGT
CACAGGGTGCCCCACGCTGCCTGCCTCTGGGCGAACACCCCATCACGCC
CGGAGGAGGGCGTGGCTGCCTGCCTGAGTGGGCCAGACCCCTGTCGCCA
GGCCTCACGGCAGCTCCATAGTCAGGAGATGGGGAAGATGCTGGGGACA
GGCCCTGGGGAGAAGTACTGGGATCACCTGTTCAGGCTCCCACTGTGAC
GCTGCCCCGGGGCGGGGGAAGGAGGTGGGACATGTGGGCGTTGGGGCCT
GTAGGTCCACACCCAGTGTGGGTGACCCTCCCTCTAACCTGGGTCCAGC
CCGGCTGGAGATGGGTGGGAGTGCGACCTAGGGCTGGCGGGCAGGCGGG
CACTGTGTCTCCCTGACTGTGTCCTCCTGTGTCCCTCTGCCTCGCCGCT
GTTCCGGAACCTGCTCTGCGCGGCACGTCCTGGCAGTGGGGCAGGTGGA
GCTGGGGGGGGCCCTGGTGCAGGCAGCCTGCAGCCCTTGGCCCTGGAGG
GGTCCCTGCAGAAGCGTGGCATTGTGGAACAATGCTGTACCAGCATCTG
CTCCCTCTACCAGCTGGAGAACTACTGCAACTAGACGCAGCCCGCAGGC
AGCCCCACACCCGCCGCCTCCTGCACCGAGAGAGATGGAATAAAGCCCT
TGAACCAGC

### Example 24: The Use of Treated Cells for the Production of Bioactive Compounds and Antibiotics

Penicillium spp. was incubated for 10 days at +25-27 C on a Sabouraud medium.

Fungi were treated with multiple rounds of DNase and RNase (“-T”) or with PBS (“—C”) as previously described or grown on agar supplemented with RNase 1 (—R) (FIG. 8).

The volume of exudate was analyzed after 4 days of culturing. Also, the properties of guttation product produced by treated cells was evaluated as the Minimal Inhibitory Concentration studied as the minimal dilution of guttation product capable to completely inhibit growth of bacteria and fungi. The treated cells produce more products and these products possess unique characteristics not identical to one seen on the original non-treated cells, sharing the similarity with evolutionary old ancestors.

### Example 25. Use of Treated Cells for Recombinant Protein Production in Bacteria

An E. coli clone producing human IL-2 was used, treated with DNase and RNase for treatment (“—T”) or PBS as control (“—C”), and grown in ampicillin. Recombinant protein was induced with 1 mM IPTG, cells were lysed, and centrifuged at 10,000 g for 20 minutes at +4° C. to isolate IL-2 inclusion bodies. The yield from E. coli-T was 6.15 fold higher (p<0.05) compared with E. coli-C.

The data received indicate that treated bacteria produce significantly higher amounts of recombinant proteins.

### Example 26. The Use of Food Treated Cells in Food and Beverage Production

We studied how treated strains of Lactobacillus delbrueckii (LD), Lactobacillus plantarum (LP), Streptococcus thermophilus (ST), and Bifidobacterium breve (BB) could improve the properties of milk products, particularly yogurt. The bacterial cultures were either treated with multiple rounds of DNase and RNase, marked as “-T” (treated), or left untreated, marked as “—C” (control).

The cow milk was pasteurized, cooled to 106 F degrees, and inoculated with the bacterial starting cultures. The survival was assessed after 28 days of storage at +4° C. (Table 38).

TABLE 38
Survival of LP in Various Yogurt Compositions.
Viability compared to the initial concentration set at 100%

| LD-C | LD-T | +ST-C | ST-T | LD-C | LD-C | BB-C | BB-T |
|---|---|---|---|---|---|---|---|
| 0.09% | 21%* | 9%* | 59%* | 0.04% | 41%** | 4% | 81%** |

*p < 0.05 -T compared to -C

Treated cells were able to survive digestion under conditions of human gastric stress. In low pH (pH 3) environments LP-C dropped by 3.4 log 10 cells/ml and LP-T by 0.2 log 10 cells/ml (p<0.05).

Subsequently, we analyzed the impact of treatment Lactobacillus rhamnosus (LR) by subjecting it to multiple treatments with benzonase, thereby generating LR-T, on cheese properties and fermentation speed. Volatile compounds in milk fermented with LR-T were identified after 120 hours at +37° C., using solid-phase extraction mass spectrometry (Agilent Technologies) (Table 39). Significant shifts were observed in some of these compounds, as detailed in Table 39.

TABLE 39
Concentrations and Significant Shifts in Volatile Compounds Produced by LR-T.
Increase in concentration difference between LP-T and LP-C*

| 1 | 2 | 3 | 4 | 5 | 6 | 7 | 8 | 9 | 10 | 111 | 12 | 13 |
|---|---|---|---|---|---|---|---|---|---|---|---|---|
| 5.9 | 2 | 2.2 | 2.8 | 2.9 | 2.2 | 2.7 | 12 | 3 | 3.9 | 1.8 | 6.8 | 2.1 |

*All p < 0.05
**1- acetic acid; 2-2-methyl-propanoic acid; 3-butanoic acid; 4 -; 2-methyl-hexanoic acid; 5- hexanoic acid; 6- octanoic acid; 7-2-octanol; 8 - 3-methyl-3-buten-1-ol; 9 -3-methyl-2-buten-1-ol; 10- octanoic acid ethyl ester; 11- decanoic acid ethyl ester; 12 - acetoin; 13-2-nonanone.

Also, the minimal fermentation time was reduced by 72% (p<0.05) by using treated starting culture.

Evaluated brewing performance of Saccharomyces cerevisiae VT-009 (SC) as a starter culture. Growth of control (SC-C) and treated (SC-T) strains was tested on YPD Medium with 0-20% ethanol, measuring optical density at OD600 (Table 40 below).

TABLE 40

| | Cell growth at 24 hours under varying | | | | |
|---|---|---|---|---|---|
| SC | ethanol (v/v) concentrations (A600) | | | | |
| strain | 0% | 8% | 10% | 15% | 20% |
| SC-C | 1.8 | 0.8 | 0.3 | 0.2 | 0.2 |
| SC-T | 1.7 | 1.7* | 1.5* | 1.3* | 1.1* |

*p < 0.05

The results demonstrated that SC-T strains are ethanol-tolerant, making them suitable for high-gravity brewing and the production of alcohol products with novel properties.

### Example 27. The Use of Treated Organisms in Remediation, Inactivation of Xenobiotics Including Heavy Metals

We assessed the bioaccumulation potential of treated cells for heavy metal remediation using Escherichia coli (EC) VT-17 and Bacillus subtilis (BS), treated with DNase and RNase, or antibodies against DNA/RNA. Treated cells were designated as “-T1” (EC-T1), “—Ta” (EC-Ta), and “-Tr” (EC-Tr), while control cells were treated with PBS (“—C” EC-C). We tested heavy metal concentrations: Cd (up to 50 mg/L), Cr (up to 40 mg/L), Pb (up to 100 mg/L). Specifically, we evaluated treated E. coli's ability to remediate mercury by inoculating EC-C and EC-T in LB broth with varying Hg2+ concentrations, incubating for 24 hours at +37° C. with shaking, then measuring bacterial growth and residual Hg2+ spectrophotometrically (Table below). Bioabsorption capacity of treated cells

TABLE 41

| | Residual Hg2+ | | | | |
|---|---|---|---|---|---|
| | concentration (mg/L) | | Bioabsorption (%) | | |
| | following Hg2+ | | Cd 50 | Cr 40 | Pb 100 |
| Probe | 5.00 mg/L exposure | Probe | mg/L | mg/L | mg/L |
| EC-C | 4.8 | BS-C | 0%* | 0%* | 0%* |
| EC-T | Below detection limit* | BS-T | 88%* | 94%* | 91%* |
| EC-Ta | Below detection limit* | — | — | — | — |
| EC-Tr | Below detection limit* | — | — | — | — |

*p < 0.05

### Example 28. The Use of Treated Cells in the Inactivation and Remediation of Xenobiotics

We assessed the efficacy of DNase and RNase-multiple times-treated Bacillus licheniformis (VT-260, BL-T) versus PBS-treated control (BL-C) for atrazine remediation. Cultures (1×10\hat{ }5 cells/mL) in M9 or LB broth with 50 mg/L atrazine were incubated at 30° C., shaken at 125 r/min for 96 hours, and atrazine levels were measured bi-hourly by HPLC-MS/MS (Table 42).

TABLE 42
Degradation of Atrazine by Treated Bacteria.

Atrazine(mg/L)

| Probe | 48 h | 96 h |
|---|---|---|
| BL-C | 47 | 45 |
| BL-T | 12* | 0* |

*p < 0.05

### Example 29. Treatment of Bacterial Expression System of the Pyocyanine Product

Pseudomonas aeruginosa SUS-24 isolates were cultured in GRM broth with 100 μg/mL nuclease at 37° C. for 72 hours for pigment production. The pigment-rich culture was centrifuged (10000 rpm, 15 min), and the supernatant used as a crude extract. Pigment was extracted using chloroform (2:1 ratio to broth), forming a blue layer, then acidified with 0.1 N HCl (20% volume), turning pink after centrifugation. The pink layer was neutralized with Tris-Base, pH adjusted, and MIC evaluated as previously described.

Results on the produced compound on microbial growth are set forth in the Table 43 below.

TABLE 43

| | | |
|---|---|---|
| Dilution of the product | Control | DNase supplemented |

S. aureus SA 2098 (OD570)

| | | |
|---|---|---|
| 1/4 | 1.234 | 0.558* |

E. coli 47 (OD570)

| | | |
|---|---|---|
| 1/4 | 1.277 | 0.206* |

*p < 0.05

Data received indicate that products produced by the cells cultivated in the presence of nucleases possess higher level of biological activity.

### Example 30. Use of Treated Cells in Plant Bioproduction

To analyze the impact of treated cells on plant growth, we utilized Bacillus subtilis (BS) that had been subjected to multiple rounds of DNase and RNase treatment. These treated cells were then incorporated into the irrigation water at a concentration of 10\hat{ }7 CFUs per gram of soil.

The results are presented in Table 44 below.

TABLE 44
Growth Promotion of Solanum melongena
Using Treated Bacillus subtilis Cell.

| Probe | Stem height | Leaves per plant |
|---|---|---|
| BS-C | 13 ± 4 | 6 ± 2 |
| BS-T | 22 ± 2* | 11 ± 1* |

*p < 0.05

### Example 31. The Use of Treated Cells to Increase Productivity of Hybridomas

Mouse Anti-Human EGFR Hybridoma [mAb 219, IgG2a kappa] from Creative Diagnostics, developed from animals immunized with EGFR cDNA-transfected CHO cells and fused with NS-1 mouse myeloma cells, targets human EGFR and inhibits tumor cell growth. Hybridoma cells, untreated or DNase/RNase-treated (treated), were cultured at 37° C., 5% CO2, with 2-3×10\hat{ }5 cells/mL. Antibody levels were measured by ELISA, with cell count/viability via Acridine Orange. Treated cells showed increased numbers, leading to recalculated antibody production per cell, with control as 100% reference. Results in the Table 45 below.

TABLE 45
Antibody Production by Hybridoma Cells.

| | Mean of antibodies |
|---|---|
| Probe | production by a single cell |
| Control hybridoma | 100% |
| Treated hybridoma | 247% ± 38%* |

*p < 0.05

### Example 32. The Use of Treatment of Eukaryotic Multicellular Organisms to Increase Productivity of the Desired Product

We used commercially purchased eggs of Bombyx mori (BM), randomized them into two groups and treated one of them with multiple rounds of DNase and RNase to obtain treated eggs (BM-T) or left untreated (BM-C). Both BM-T and BM-C were maintained under the same conditions. We analyzed the weights of cocoons on the 3^{rd} day of their formation using a laboratory balances (Mettler Toledo). The data were statistically analyzed and presented in the Table 46 below.

TABLE 46
Cocoon characteristics of BM-C and BM-T.
Cocoon (g)

| BM-C | BM-T |
|---|---|
| 1.18 ± 0.15 | 1.99 ± 0.11* |

*p < 0.05

### Example 33: The Use of Cell Treatment to Regulate Cell Expansion and Differentiation

PBMCs were sourced from five healthy volunteers and T cells isolated via Ficoll centrifugation. Control T cells received PBS (T4-C); experimental ones were treated with DNase and RNase (T4-T). T cell activation/expansion used Miltenyi Biotec's kit with varying bead-to-cell ratios, removing beads on day 7.

Experiments assessed IL-2's impact on expansion at five concentrations (20-500 IU/mL) and seeding density's effect at five densities (6×10\hat{ }4 to 2.5×10\hat{ }6 cells/mL), both using a 1:2 bead-to-cell ratio. Additionally, the bead-to-cell ratio's impact was tested at ratios from 1:10 to 10:1, all with IL-2 at 50 IU/mL. T cell treatment notably enhanced expansion across setups.

TABLE 47
Impact of Cell Treatment on Expansion
with Various IL-2 Concentrations.

| IL concentration | Cumulative population doubling (±SD) by day 28 | |
|---|---|---|
| (IU/mL) | T4-C | T-4T |
| 20 | 4.3 ± 0.2 | 7.6 ± 0.3* |
| 500 | 9.1 ± 0.3 | 20.5 ± 1.4*** |

*p < 0.05
***p < 0.001

TABLE 48
Effect of Cell Treatment on Expansion
Depending on Various Cell Densities.

| Cell concentration | Cumulative population doubling (±SD) by day 28 | |
|---|---|---|
| (cell/mL) | T4-C | T-4T |
| 6 × 10\hat{ }4 | 6.3 ± 0.3 | 18.3 ± 0.5*** |
| 2.5 × 10\hat{ }6 | 5.2 ± 0.6 | 17.4 ± 1.4*** |

***p < 0.01

TABLE 49
Impact of Cell Treatment on Expansion
Depending on Various B:C Ratios.

| Cell concentration | Cumulative population doubling (±SD) by day 28 | |
|---|---|---|
| (cell/mL) | T4-C | T-4T |
| 1:10 | 6.9 ± 0.7 | 12.9 ± 1.2*** |
| 10:1 | 14.5 ± 0.6 | 28.1 ± 3.2*** |

***p < 0.01

### Example 34. Use of Cell Treatment in Baculovirus System

Spodoptera frugiperda 9 cell line (ATCC CRL-1711) was cultivated according to the instructions at 27° C. in Sf-900 II SFM medium in suspension. Control cells were left untreated and experimental were either treated with several rounds of DNase, RNase and their combinations or were cultivated in the presence of these nucleases from 1 to 30 g/mL.

The neuraminidase gene of influenza A virus was subcloned into a pIZ/V5 His (Invitrogen, Thermo Fisher Scientific, Inc., USA) and further transfected into Groups 1-7 of Sf9 cells using Cellfectin II (Invitrogen) according to the manufacturer instruction.

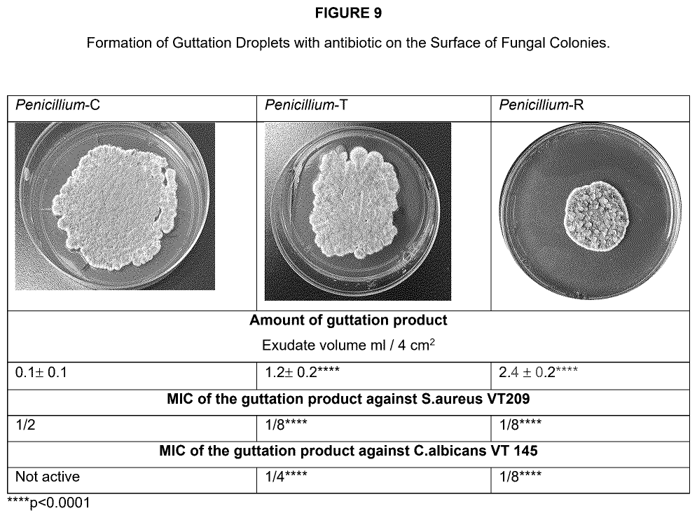

The results are shown in FIG. 9.

Data received indicate that treatment cell culture conditions was able to increase the yield of products synthesized in insect cells, including virus-like particles produced by the baculovirus expression system.

### Example 35. Use of Treated Cells to Generate Products with New Characteristics

To study effects of nucleases use on organoleptic properties of bread, Saccharomyces cerevisiae were pretreated with one (×1) or several times (Treated) with DNase I and RNase taken at 1 μg/mL, washed from nucleases and used in bread baking. The results are set forth in Table 49 below.

TABLE 49

| | | Crumb | | | Moisture |
|---|---|---|---|---|---|
| | Crust color | softness and | Flavor | | content and |
| Parameter | and texture | elasticity | profile | Aroma | shelf life |
| Control | 6.0 | 5.0 | 6.0 | 5.0 | 6.0 |
| X1 | 9.0 | 7.0 | 8.0 | 7.0 | 8.0 |
| Treated | 8.0 | 9.0 | 9.0 | 8.0 | 8.0 |

Bread obtained from “×1” and Treated fungi are richer, more uniform golden-brown crust with a crisper texture, enhanced softness and elasticity, pillowy texture. Notably, ×1 and treated bread exhibits a delicate hint of olive oil and rosemary. Additionally, an unexpected but pleasant slight sweetness is detected, reminiscent of honey. The aroma is markedly more complex and inviting. One is greeted with the rich scent of toasted almonds and a subtle hint of vanilla. This bouquet of aromas is enriched further by a slight citrus undertone.

Next, we treated tomato seeds to generate new tomatoes. To study effects of nucleases use on plants tomato seeds were pretreated with DNase I and RNase A once (×1) or with multiple times (Treated), washed from nucleases and sown in plastic trays and were transplanted with a single seedling in three liter capacity plastic pots filled with compost. The experiment was carried out in greenhouse with the medium temperature 22° C. and 34% humidity. We will assign scores to each organoleptic property of both control tomatoes (Control) and treated tomatoes (×1 and Treated), on a scale from 0 to 10, where 0 represents the lowest quality and 10 represents the highest quality.

Data are shown in Table 50 below.

TABLE 50

| | Color | | | | |
|---|---|---|---|---|---|
| | Intensity and | | Flavor | | Juici- |
| Parameter | Uniformity | Texture | Profile | Aroma | ness |
| Method | ColorFlex EZ | Pene- | Content of | GC-MS | Weight |
| | Tomato | trometer | sugars by | | of |
| | | 53505/FB | Bertrand's | | juice |
| | | | method | | |
| Control | 6.0 | 5.5 | 6.0 | 5.5 | 6.0 |
| x1 | 8.5 | 8.0 | 9.0 | 8.5 | 8.0 |
| Treated | 9.0 | 9.0 | 10.0 | 9.0 | 9.0 |

“×1” and Treated tomatoes exhibited a deeper and more uniform red color compared to the T group. The color intensity, measured by a spectrophotometer, showed a significant increase in the ×1 and Treated groups, indicating enhanced lycopene content.

The texture of ×1 and Treated tomatoes was significantly improved, with a firmer and more consistent flesh. This was quantified using a penetrometer, where ×1 and Treated tomatoes required higher force to penetrate, suggesting enhanced cell structure and firmness.

Sensory analysis revealed that Treated tomatoes had a more pronounced flavor profile. The most notable achievement was the development of a unique spicy flavor profile in the tomatoes. Tasting panels reported a mild to moderate heat level, reminiscent of the sensation provided by mild chili peppers.

The ×1 and Treated tomatoes in particular, emitted a stronger and more appealing tomato aroma, which was confirmed through gas chromatography-mass spectrometry analysis. Volatile compounds associated with the characteristic tomato aroma were found in higher concentrations in the treated groups. The tomatoes exhibited a distinctive aromatic profile with clear notes of rosemary and thyme.

X1 and Treated tomatoes were found to be juicier than their control counterparts. This was measured by the weight of juice extracted from an equal weight of tomatoes, with ×1 and Treated tomatoes producing an average of 10-20% more juice.

### Example 36. The Use Treated Cells for the Treatment of Inflammatory Diseases

Crohn's disease and ulcerative colitis are the major types of inflammatory bowel diseases (IBD). To model them, we used the 2,4,6-trinitrobenzene sulphonic acid (TNBS) model.

Briefly, C57BL/6J mice (both females and males) were anesthetized with xylazine and ketamine, and a TNBS solution was prepared in absolute ethanol to a final concentration of 2.5% TNBS in 50% ethanol. To induce IBD, the mice were administered 20 μl of 2.5% TNBS in 50% ethanol (5 μl/g body weight) rectally using a gavage needle. The disease activity index was calculated by analyzing stool consistency, rectal bleeding, and the percentage of weight loss during the observational period.

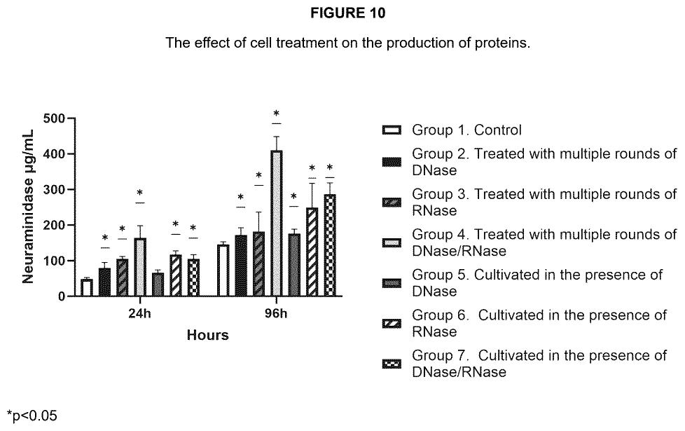

Mice were either left untreated or treated (control #1) with SL4-C (Mock-control #2) or SL4-T (experimental group). SL4-T cells were obtained as previously described. SL4-T and SL4-C were administered to the mice once a week at concentrations ranging from 10\hat{ }3 to 10\hat{ }7 cells per mouse, either intravenously (SL4-Civ; SL4-Tiv) or as a rectal instillation (SL4-Crec: SL4-Trec) (see FIG. 10 below).

Data received demonstrated that SL4-T can be used for the treatment of inflammatory bowel diseases and are effective through different routes of administration.

### Example 37: The Use of Treated Cells for the Treatment of Diseases Associated with Protein Misfolding

We investigated the clearance of misfolded, prionogenic proteins implicated in neurodegenerative and autoimmune diseases by comparing treated cells to controls. Cells were treated through treatments with DNase, RNase, or anti-DNA/RNA antibodies. Primary neuronal cultures were derived from rat embryo hippocampi.

Studies involved prion-like proteins (Tau, beta-amyloid, α-synuclein, SOD1, TDP-43, IAPP) linked to diseases like Alzheimer's and Parkinson's. Proteins, in monomeric form, were aggregated using heparin and incubated at 37° C., with aggregation tracked by Thioflavin T fluorescence and PMCA method. 10\hat{ }8 of the tested cells were tested with 100 nM of aggregated proteins to assess clearance efficiency, detailed in Table 51.

TABLE 51
Clearance of misfolded proteins with treated cells following 120 min contact time.

| | % misfolded protein uptake | | | | | |
|---|---|---|---|---|---|---|
| | | Beta- | Alpha- | | | |
| Probe | Tau | amyloid | synuclein | SOD1 | TDP43 | IAPP |
| Control | 2 ± 1 | 3 ± 1 | 3 ± 1 | 0 | 1 ± 1 | 4 ± 3 |
| Agranulocytes | 0 | 5 ± 4 | 4 ± 3 | 2 ± 1 | 6 ± 1 | 5 ± 1 |
| (untreated) | | | | | | |
| Agranulocytes + platelets | 3 ± 2 | 7 ± 6 | 0 | 0 | 4 ± 2 | 7 ± 3 |
| WBC + platelets | 6 ± 5 | 3 ± 2 | 0 | 5 ± 3 | 4 ± 2 | 3 ± 1 |
| (untreated) | | | | | | |
| Neural cells (untreated) | 3 ± 2 | 4 ± 1 | 5 ± 1 | 0 | 0 | 0 |
| AGR-T (treated) | 43 ± 12* | 42 ± 24* | 34 ± 19* | 41 ± 15* | 55 ± 17* | 40 ± 23* |
| SL2-T (treated) | 69 ± 24* | 73 ± 19* | 42 ± 18* | 55 ± 9* | 68 ± 13* | 56 ± 14* |
| SL4-T (treated) | 86 ± 23* | 91 ± 17* | 75 ± 16* | 89 ± 10* | 92 ± 13* | 68 ± 9* |
| SL4-Ta (treated with | 82 ± 12* | 85 ± 13* | 59 ± 10* | 74 ± 18* | 85 ± 22* | 61 ± 8* |
| antibodies) | | | | | | |
| Neural cells (treated) | 39 ± 7* | 49 ± 12* | 22 ± 7* | 35 ± 10* | 17 ± 9* | 31 ± 11* |

*p < 0.05

The data indicates that treated cells have a higher capacity to remove misfolded aggregates.

### Example 38. The Use of Treated Cells in Patients with Psoriasis

We examined the impact of treated SL4-T cells on psoriasis patients with baseline PASI scores >12. Using autologous cells, each patient served as their own control due to demographic diversity.

Group A: 1×10\hat{ }8 cells/patient i.v., bi-weekly; Group B: 1×10\hat{ }4 cells/patient i.v., every other day; Group C: 1×10\hat{ }8 cells/patient i.v., every other day; Group D: 1×10\hat{ }8 cells in 10 ml baby cream, topically every other day.

Patients were monitored for up to 12 months post-therapy, during which they experienced no exacerbations or new lesions, contrasting with their history of up to 2 exacerbations annually (Table 1).

TABLE 52
Comparison of the number of exacerbations
of psoriasis during 12 months follow-up.

| Treatment | 12 months prior to the use of | 12 months after the |
|---|---|---|
| Group | treated cells | use of SL4-T cells |
| A | 2 | 0 |
| B | >2 | 0 |
| C | 2 | 0 |
| D | >2 | 0 |

Data received indicate that the use of treated cells can be used for the successful management of psoriasis.

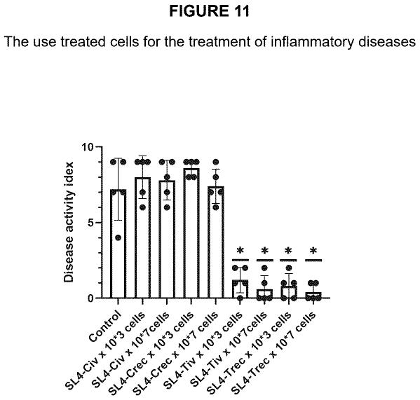

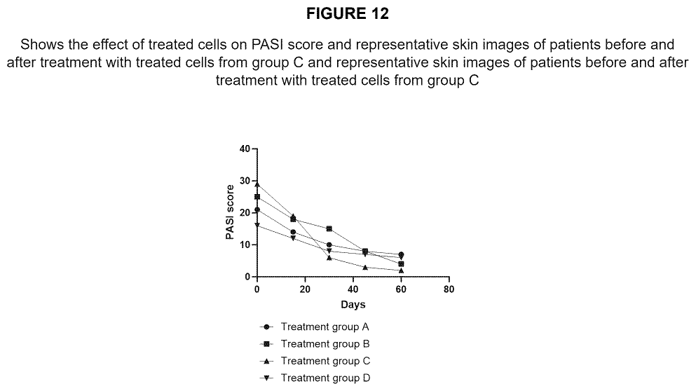

FIGS. 11 and 12 show the effect of treated cells on PASI score and representative skin images of patients before and after treatment with treated cells from group C and representative skin images of patients before and after treatment with treated cells from group C.

### Example 39. The Use of Treated Cells for the Treatment of Diabetes

To study diabetes modulation, C57BL/6 mice were fasted for 12 hours. The mice were then injected intraperitoneally with 60 mg/kg STZ in citric acid buffer (pH 4.5) for five days. Mice with fasting blood glucose over 300 mg/dl were deemed diabetic.

Mesenchymal stem cells (MST), induced pluripotent stem cells (iPSC), and primary fibroblasts (FB) were treated via multiple DNase, RNase, or anti-DNA/RNA antibody treatments, following standard procedures.

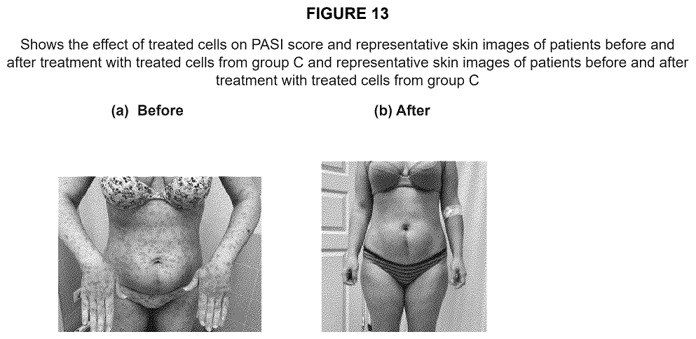

Mice groups were: untreated control (C), mesenchymal stem cells control (MST-C) and treated (MST-T), iPSC control (iPSC-C) and treated (iPSC-T), and fibroblasts control (FB-C) and treated (FB-T), all injected with 1×10\hat{ }6 cells in 300 μL of DMEM or RPMI 1640 into the hepatic portal vein. Blood glucose was monitored every five days (FIG. 13).

### Example 40. Effect of Treated Cells in Patients with Autoimmune Diseases Associated with the Elevated Levels of Autoantibodies

A 52-year-old woman presented with Hashimoto thyroiditis with the presence of thyroid peroxidase antibodies anti-TPO. The patient was referred for the treatment with allogeneic SL4-T cells obtained as previously described. SL4-T and MSC-T cells were given as i.v. injection at concentration 10*7 cells/injection once a week. The results are shown in table 1.

TABLE 53
Level of autoantibodies during the therapeutic course.

| Week post therapy | Anti-TPO |
|---|---|
| initiation | level |
| 0 | 196 |
| 6 | 41* |

*p < 0.05

Next, we analyzed the effect of autologous SL4-T for the treatment of diabetes (type 1 diabetes) in four patients with diabetes confirmed at least over 3 years prior to the initiation of the study. Allogeneic, treated SL4-T cells were obtained as previously described. Human mesenchymal stem cells were treated following eight cycles of treatment with DNase and RNase (MSC-T) each taken at concentration 50 mg/L. Cells at the concentration of 10*6 cells/injection were injected weekly for 3 months (Table 54below).

TABLE 54

| | | Baseline data before therapy | | | 3-month post therapy | | |
|---|---|---|---|---|---|---|---|
| Therapeutic | | ICA | IA2 | GAD | ICA | IA2 | GAD |
| agent | Patient | (JDF units) | (units/ml) | (units/ml) | (JDF units) | (units/ml) | (units/ml) |
| SL4-T | 1 | 14 | 125 | 165 | 0* | 5* | 25* |
| SL4-T | 2 | 18 | 150 | 275 | 3* | 0* | 35* |
| MSC-T | 3 | 22 | 100 | 150 | 1* | 10* | 10* |
| MSC-T | 4 | 11 | 105 | 110 | 0* | 0* | 10* |

*p < 0.05

ICA—islet cell antibodies; IA2—antibodies against islet antigen-related tyrosine phosphatase 2; GAD-glutamic acid-decarboxylase;

At 3-month follow-up after treatment, all patients presented positive dynamics for the therapy with reduced levels of autoantibodies which were significantly elevated prior to the initiation of the study (p value <0.05). Data received show the efficacy of therapy of the autoimmune diseases with autologous and allogeneic treated cells.

### Example 41. The Use of Treated Cells for the Treatment of Systemic Lupus Erythematosus

A total of five participants with refractory systemic lupus erythematosus (SLE) were treated with treated allogeneic or autologous SL4-T cells. Disease status was assessed by evaluating anti-nuclear antibodies (ANA) using indirect immunofluorescence. SL4-T cells were administered intravenously (i.v.) in doses ranging from 10\hat{ }5 to 10\hat{ }8 cells per injection, once a week (see Table 55).

TABLE 55
Dynamics of ANA in treated patients.

| Week | Patient 1* | Patient 2* | Patient 3** | Patient 4** | Patient 5*** |
|---|---|---|---|---|---|
| 0 | 1:1280 | 1:640 | >1:1280 | >1:1280 | 1:1280 |
| 1 | 1:640 | 1:640 | 1:640 | 1:320 | 1:640 |
| 2 | 1:160 | 1:80 | 1:160 | 1:80 | 1:320 |
| 4 | 1:80 | 1:40 | 1:160 | 1:40 | 1:40 |

*i.v. injected 10*5 cells/injection;
**i.v. injected 10*7 cells/injection;
***i.v. injected 10*8 cells/injection

### Example 42. The Effect of Treated Cells on Multiple Sclerosis

Three patients with a previous history of multiple sclerosis (MS) underwent treatment with treated SL4-T cells. In two patients, SL4-T cells were allogeneic, and in one patient, they were autologous. The administration of SL4-T cells was through intravenous (i.v.) injection, following various regimens ranging from twice a week to once a month. The therapy spanned 6 months, followed by a 6-month observation period. Expanded Disability Status Scale (EDSS) and annual number of relapses. Each patient acted as their own control, comparing data from the 12 months prior to the initiation of SL4-T therapy. The results of the therapy are presented in table 57.

TABLE 56
The use of SL4-T for the treatment of MS.

| | 12 months prior to the | | 12 months after the | |
|---|---|---|---|---|
| | initiation of SL4-T therapy | | initiation of SL4-T therapy | |
| | EDSS | Number of | EDSS | Number of |
| Patient | score | relapses | score | relapses |
| 1 | 6.5 | 2 | 1.5 | 0 |
| 2 | 5.5 | 1 | 1.0 | 0 |
| 3 | 6.5 | 1 | 1.5 | 0 |

### Example 43. The Effect of Treated Cells on Neurodegenerative Disorders

When exploring the utility of a new therapeutic agent designed for treating neurodegenerative diseases, such as Alzheimer's disease, Parkison's disease, myotrophic lateral sclerosis, motor neuron disease, Huntington's disease, spinal muscular atrophy, and spinocerebellar ataxia; or neurodevelopmental conditions such as autism spectrum disorders, it is essential to evaluate its impact on behavioral and cognitive parameters that are often compromised in such conditions. Here we studied the effects of treated SL4-T or MRC-C cells on various behavioral aspects in mice.

Mice were i.v. injected with various concentrations of SL4-T or SL-C or with MRC-C MRC-T cells varying from 10*3 to 10*7 cells per mouse.

### Locomotor and Exploratory Behavior

Home Cage Activity: Baseline locomotion measured by distance covered in five minutes in familiar settings, noting changes post-treatment.

Open Field Test/Anxiety-Like Behavior: Assesses locomotion and exploration by tracking movements and zone occupancy in a square arena for five minutes.

Elevated Zero Maze (EZM): Evaluates anxiety through exploration patterns between open and closed maze quadrants over five minutes.

Open Field Test (Anxiety Context): Analyzes anxiety by measuring time spent in central versus peripheral zones.

### Spatial Learning and Memory

Barnes Maze Test: Tests spatial memory by recording escape box search efficiency, including latency and errors, influenced by treatment.

Depression-Like Behavior/Forced Swim Test (FST): Measures despair by tracking immobility duration in water, observing changes due to therapeutic intervention.

Locomotor Activity (HOME CAGE CONDITION (NON-STRESSFULL) Examined across weeks, focusing on total and center distance traveled, revealing treatment impacts.

The results are shown in Table 57 below.

TABLE 57

| | | Experimental weeks | | | |
|---|---|---|---|---|---|
| | | | Week 1 | Week 3 | Week 5 |
| | | Week 0 | 1 week | 1 week | 1 week |
| Mice | | Before | after 1^{st} | after 2^{nd} | after 3^{rd} |
| groups | Test parameters | injection | injection | injection | injection |
| Control | Total distance traveled, | 507 ± 12.3 | 479.7 ± 5 | 477 ± 7.81* | 443.3 ± 8.5* |
| | cm (10 min) | | | | |
| | Center distance, cm | 139.7 ± 9.5 | 133.3 ± 9 | 132 ± 9.2 | 123 ± 5.6* |
| SL4-T | Total distance traveled, | 536.7 ± 17.5 | 511.7 ± 18.6* | 519.3 ± 13.58 | 553.3 ± 17.9 |
| | cm (10 min) | | | | |
| | Center distance, cm | 141.3 ± 11.5 | 147 ± 11.1* | 150.7 ± 13.7 | 153.7 ± 12.5 |
| MRC-T | Total distance traveled, | 518.3 ± 15.6 | 490.3 ± 3.5* | 491 ± 10.82 | 510 ± 11.1 |
| | cm (10 min) | | | | |
| | Center distance, cm | 139.7 ± 5.7 | 142.3 ± 4.9* | 145.7 ± 4* | 152 ± 10.4 |

*p < 0.05

Anxiety-like behavior (Analyzed via open field and set forth in Table 58 below).

TABLE 58

| | | Experimental weeks | |
|---|---|---|---|
| | | Week 0 | Week 5 |
| | | Before | 1 week after 3rd |
| Mice groups | Test parameters | injection | injection |
| Control | Distance traveled | 341 ± 18.5 | 268.7 ± 11.9* |
| | in 6 minutes, cm | | |
| SL4-T | Distance traveled | 338 ± 26.5 | 378.3 ± 56.5 |
| | in 6 minutes, cm | | |
| MRC-T | Distance traveled | 322.3 ± 12.7 | 293 ± 26.6 |
| | in 6 minutes, cm | | |

*p < 0.05

Spatial Learning and Memory (Assessed with Barnes Maze Test)

Through a rigorous experimental setup, the Barnes Maze test was conducted over four consecutive days in the fifth week, following the 3^{rd} injection, to evaluate and contrast the behavioral attributes of mice exposed to various formulations. The parameters evaluated were time spent in open arms and the number of transitions, both pivotal in portraying anxiety-like behaviors and memory functions in murine models. The results are set forth in Table 59 below.

TABLE 59

| | | Barnes maze day | |
|---|---|---|---|
| Mice groups | Test parameters | 1 day | 4 day |
| Control | Open arms, s | 212 ± 39 | 183.7 ± 30.3 |
| | Transitions, number | 55 ± 3.5 | 41 ± 4.4 |
| SL4-T | Open arms, s | 195.7 ± 30 | 270 ± 26.7 |
| | Transitions, number | 57 ± 1.7 | 27.7 ± 9.3 |
| MRC-T | Open arms, s | 180.7 ± 26.9 | 253.7 ± 37.2 |
| | Transitions, number | 54 ± 2 | 32 ± 6.2 |

Depressive-like behaviour (assessed by Forced swim test.)—set forth in Table 60 below.

TABLE 60

Open arms, s

| | Week 0 | Week 5 |
|---|---|---|
| | Before | 1 week after |
| Mice groups | injection | 3rd injection |
| Control | 45.5 ± 3.2 | 61.5 ± 3.2 |
| SL4-T | 48.5 ± 2.8 | 26.2 ± 5.2* |
| MRC-T | 47 ± 3 | 27.7 ± 5.5* |

*p < 0.05

Mice treated with SL4-T exhibited a remarkable rise in time spent in open arms from, indicating reduced anxiety-like behavior.

### Example 44. The Use of Treated Cells for the Treatment of Rheumatoid Arthritis

### Patient Details

53-old female. Diagnosis: Seronegative Rheumatoid Arthritis, ACPA-Positive (6.2), Late Stage, Erosive, Radiological Stage 3, Activity 3, Functional class 3.

### Pre-Treatment Status.

Symptoms: Pain and stiffness in the joints, Chronic fatigue, Prolonged morning stiffness, Symmetrical joint swelling, Reduced range of motion, Development of rheumatoid nodules, Persistent low-grade fever, Unintended weight loss, Elevated inflammatory markers. Prior Treatment: DMARDs (Disease-Modifying Antirheumatic Drugs): Methotrexate was administered to manage inflammation and modulate the autoimmune response. Corticosteroids: Prednisone was employed to manage acute flares and to potentially decelerate joint damage. Physical Therapy: Joint exercises and mobility training.

Despite these comprehensive interventions, the patient demonstrated no significant improvement, thereby necessitating exploration into alternative therapeutic approaches.

Treatment Plan SL4-T was administered intravenously once a week at the concentration of 10*7 cells/injection

### Treatment Course and Outcomes (2 Months)

Observations showed a gradual decrease in morning joint pain and stiffness, significant reduction in swelling of smaller joints (fingers and wrists), and enhanced joint motion and function. The patient experienced better sleep, less fatigue, and increased physical ability.

CT scans one-month post-treatment displayed improvements in both knees, with reduced bone marrow edema and osteochondral damage, and less synovial fluid and popliteal cyst size.

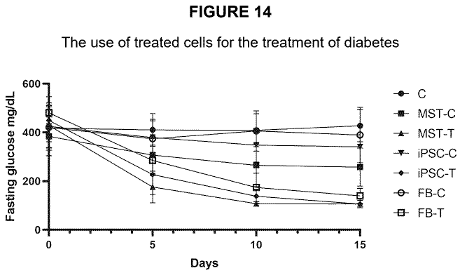

Overall, the patient noted a substantial improvement in life quality, symptom relief, and functionality. The results are presented in FIG. 14.

### Example 45: Use of SL4-T for the Therapy of Neurodegenerative Diseases

### History of Present Illness:

Mr. B, a 40-year-old IT specialist, began experiencing significant memory issues 18 months ago, initially attributing them to stress. His condition evolved into marked cognitive and motor impairments, including forgetfulness, difficulty focusing, and name recall challenges. He developed a shuffling gait and physical instability, reducing his mobility and independence, alongside involuntary twitching and occasional blurred vision. The patient had confusion and disorientation. Pronounced muscle weakness. Marked walking instability, needing support. Motor agitation and involuntary finger movements. Gait apraxia with a rightward lean and excessive foot dorsiflexion.

### Physical Examination Upon Admission:

Cognitive Status: Deteriorated; showed issues in immediate and short-term memory, difficulty concentrating, and slowed problem-solving abilities.

Motor Skills: Noticeable motor restlessness, impaired coordination, an unstable, shuffling gait.

Neurological Findings: Subtle dysarthria, nystagmus, and a positive Romberg sign.

### Laboratory Tests & Imaging:

Signs of atrophy in the cerebellum and changes in the basal ganglia. Normal metabolic blood panel, negative for common infectious and autoimmune markers. Normal pressure, no infectious or malignant cells.

### Initial Treatment Approach:

Donepezil (5 mg/day), Memantine (10 mg twice daily); Levodopa/Carbidopa (Sinemet); Pramipexole (0.125 mg three times a day); Baclofen (5 mg×3 times a day); Sertraline (50 mg/day); Quetiapine (25 mg at night, titrated cautiously). Supportive Therapy: Speech and Language Therapy;

### Nutritional Management

### Experimental Therapy Initiation:

Despite ongoing treatment, the patient's symptoms worsened. Due to limited conventional therapy response, an experimental SL4-T regimen was initiated with consent, ranging from weekly to daily injections of 10\hat{ }7 or 10\hat{ }8 cells.

### Outcome Post-SL4-T Therapy:

### 2 Months Post-Therapy:

Cognitive function significantly improved; FAB score increased from 5/18 to 11/18, and MMSE score from 9/30 to 19/30, indicating better cognitive flexibility, orientation, and speech.

Motor symptoms, including shuffling gait and twitching, showed marked improvement.

Quality of life improved with enhanced cognitive and motor functions, leading to greater independence and social interaction.

### SL4-T Therapy Effects:

Targeted multiple symptoms including cognitive, motor, and oculomotor disturbances by modulating affected neural networks. Exhibited neuroprotective properties to slow neurodegenerative disease progression by protecting neurons. Enhanced cognitive functions, improving memory and attention through neural circuit modulation.

Improved motor functions, aiding in the stabilization and potential improvement of extrapyramidal and cerebellar symptoms.

Managed oculomotor symptoms, supporting eye movement control essential for independence and quality of life.

### Example 46. The Use of Treated Cells for the Treatment of Autoimmune Reactions

### Experiment 1: Evaluating SL4-T Efficacy During Anaphylactic Shock in Mice

Species: BALB/c mice, 8-10 weeks, Under Specific Pathogen Free conditions, adhering to scientific research animal care guidelines Anaphylactic Shock was induced by ovalbumin, Intraperitoneal injection, 100 μg/mouse.

Experimental Groups: CN (Control Normal, control group of healthy mice); Control Group 1 (CG1): No treatment post-anaphylactic shock induction; Control Group 2 (CG2): epinephrine, 0.01 mg/kg, intraperitoneally; SL4-C Group: SL4-C administration; SL4-T Group: SL4-T administration. All treatments are administered intraperitoneally, 10 minutes post-reaction onset.

Symptomatic Evaluation Scale: 0: No symptoms; 1: Erythema, urticaria; 2: Increased motor activity; 3: Trembling, distress; 4: Spasms; 5: Loss of consciousness; 6: Death

TABLE 61
Average Symptom Scores (mean ± SD, n = 5).

| Time | | | | | |
|---|---|---|---|---|---|
| (min) | CN | CG1 | CG2 | SL4-C Group | SL4-T Group |
| 15 | 0 ± 0 | 2.2 ± 0.7* | 1.8 ± 0.2* | 2.4 ± 0.5* | 1.2 ± 0.2 |
| 30 | 0 ± 0 | 3.1 ± 0.3* | 1.6 ± 0.6* | 3.2 ± 0.3* | 1.0 ± 0.2 |
| 120 | 0 ± 0 | 5.0 ± 0.4* | 0.7 ± 0.2* | 5.0 ± 0.5* | 0.3 ± 0.3 |
| 180 | 0 ± 0 | 5.0 ± 0.4* | 0.5 ± 0.1* | 5.0 ± 0.2* | 0.2 ± 0.1 |

*p < 0.05 compared to healthy control

TABLE 62
Immunological Parameters (mean ± SD, n=5).

| | | | | SL4-C | SL4-T |
|---|---|---|---|---|---|
| Parameter | Healthy | CG1 | CG2 | Group | Group |
| IgE (ng/ml) | 23 ± 3 | 745 ± 154 | 656 ± 126 | 693 ± 136 | 356 ± 71* |
| IL-4 (pg/mL) | 16 ± 2 | 226 ± 43 | 213 ± 39 | 215 ± 47 | 112 ± 26* |
| IL-5 (pg/mL) | 11 ± 4 | 276 ± 52 | 234 ± 44 | 242 ± 42 | 126 ± 34* |
| IL-13(pg/mL) | 12 ± 4 | 205 ± 46 | 197 ± 32 | 197 ± 44 | 104 ± 22* |
| Histamine (nM) | 53 ± 8 | 2135 ± 408 | 1814 ± 354 | 1915 ± 389 | 1211 ± 212* |
| Prostaglandins | 112 ± 11 | 4241 ± 813 | 3743 ± 751 | 3825 ± 762 | 2422 ± 456* |
| (pg/mL) | | | | | |

*p < 0.05 compared to CG1, CG2, SL-C

### Experiment 2: Evaluating SL4-T Efficacy During Delayed-Type Hypersensitivity in Mice

Delayed-Type Hypersensitivity was induced by intradermal injection of Tuberculin PPD 50 μl

### Treatment Protocols

CG1: No treatment; CG2: Dexamethasone administration (5 mg/kg), intraperitoneally, every 24 hours; SL4-C Group: SL4-C therapy, intraperitoneally, every 24 h; SL4-T Group: SL4-T therapy, intraperitoneally, every 24 h

TABLE 63
Edema Tissue Thickness Changes.

| | | | | SL4-C | SL4-T |
|---|---|---|---|---|---|
| Time (hr) | CN | CG1 | CG2 | Group | Group |
| 24 | 0 ± 0 | 2 ± 0.4 | 1 ± 0.2 | 1.7 ± 0.3 | 0.9 ± 0.3* |
| 48 | 0 ± 0 | 7 ± 0.8 | 2 ± 0.1 | 4.2 ± 0.4 | 1.1 ± 0.2* |
| 72 | 0 ± 0 | 8 ± 0.7 | 5.2 ± 0.3 | 5.8 ± 0.4 | 1.3 ± 0.2* |

*p < 0.05 compared to CG1, CG2, SL-C

TABLE 64
Cytokine Levels (pg/ml).

| | | | | SL4-C | SL4-T |
|---|---|---|---|---|---|
| Cytokine | CN | CG1 | CG2 | Group | Group |
| IFN-γ | 23 ± 6 | 223 ± 52 | 87 ± 21 | 163 ± 41 | 77 ± 13* |
| IL-2 | 17 ± 5 | 212 ± 46 | 96 ± 24 | 157 ± 32 | 76 ± 19* |
| TNF-α | 22 ± 5 | 243 ± 62 | 118 ± 32 | 188 ± 42 | 82 ± 21* |

*p < 0.05 compared to CG1, CG2, SL-C

SL4-T administration significantly reduced edema, swelling, and inflammation in delayed-type hypersensitivity, suggesting a novel approach for treating allergic contact dermatitis.

### Example 47. Effect of Cell Treatment on Inflammasome Activation

The effect of treated SL4-T cells on the activation of the NLRP3 inflammasome, which is a key pathological process in a number of disorders such as focal segmental glomerulosclerosis, ankylosing spondylitis, gout, rheumatoid arthritis, type 2 diabetes, atherosclerosis, psoriasis, and non-alcoholic fatty liver disease, was tested in bone marrow-derived macrophages (BMDMs). BMDMs were placed in minimal media in 96-well plates at 10\hat{ }5 cells/well and were first primed with lipopolysaccharide (from E. coli). Then, SL4-T or SL4-C were added at a 1:1000 ratio, and finally, NLRP3 was stimulated with ATP. SL4-T inhibited the release of IL-1β in BMDMs and PBMCs, while SL4-C did not. Notably, the viability of BMDMs was not affected by the addition of SL4-T/C as shown in Table 65 below.

TABLE 65

| Untreated | SL4-C (1:1000) | SL4-T (1:1000) |
|---|---|---|
| 0 | 2096 ± 403 | 162 ± 109* |

*p < 0.05 of SL4-T compared with SL4-C

### Example 48. The Use of Treated Cells for the Transplantation to Relative and Non-Related Organisms

We analyzed how treatment of cells could increase their potential for being used for the regenerative medicine including tissue engineering and bioprinting.

For that we analyzed how the treatment of cells could change their genes including the expression and interaction with the antibodies of the MHC class I proteins as well as safe administration for HLA-non-matched subjects and non-relative organisms.

SL4-T has Altered Interaction with Anti-HLA and Anti-CD Antibodies and Altered Expression of CD and HLA Antigens

### Materials and Methods Summary:

SL4-T cells and SL4-C controls were prepared as previously described. White blood cell counts were done using a NucleoCounter NC-202. Cells were stained with various fluorescent dyes for analysis. T- and B-cell populations were quantified using a Cytomics FC500 flow cytometer, with data analyzed via CXP Cytometer 2.2 and Kaluza software. HLA typing and antibody screening were conducted using Miseq Omixon, LABScan 100 Flow analyzer, and Illumina MiSeq, following specific sequencing protocols.

### Results Summary:

SL4-C and SL4-T showed comparable viability (82%-85%). However, significant differences were noted in white blood cell interactions with anti-CD and anti-HLA antibodies between SL4-C and SL4-T, with a notable reduction in SL4-T's white blood cell count (p<0.05). This trend extended to T- and B-cell anti-CD antibody reactions, with marked variations in CD4+, CD45+, CD19+, CD56+, and CD8+ cell counts, demonstrating significant changes in binding capabilities.

TABLE 66
The absolute number of T-lymphocytes capable
of binding to anti-CD antibodies.

| Antibody | SL4-C | SL4-T |
|---|---|---|
| CD45+ | 6.34 × 10\hat{ }9 ± | 2.58 × 10\hat{ }5 ± |
| | 1.78 × 10\hat{ }9 | 3.18 × 10\hat{ }5**** |
| CD3+ | 6.52 × 10\hat{ }9 ± | 1.78 × 10\hat{ }5 ± |
| | 2.07 × 10\hat{ }9 | 3.93 × 10\hat{ }5**** |
| CD4+ | 5.70 × 10\hat{ }9 ± | 1.58 × 10\hat{ }5 ± |
| | 3.29 × 10\hat{ }9 | 3.48 × 10\hat{ }5**** |
| CD19+ | 6.02 × 10\hat{ }9 ± | 1.82 × 10\hat{ }5 ± |
| | 1.94 × 10\hat{ }9 | 3.90 × 10\hat{ }5**** |
| CD56+ | 4.84 × 10\hat{ }9 ± | 1.70 × 10\hat{ }5 ± |
| | 1.66 × 10\hat{ }9 | 3.74 × 10\hat{ }5**** |
| CD8+ | 6.10 × 10\hat{ }9 ± | 1.18 × 10\hat{ }5 ± |
| | 2.60 × 10\hat{ }9 | 1.56 × 10\hat{ }5**** |

***p < 0.0001

Next, we analyzed the effect of treatment white blood cells on their interaction with anti-HLA antibodies, using three distinct methods. Luminex analysis revealed alterations in cellular reactivity to anti-HLA antibodies. The data obtained indicate that SL4-T cells were characterized by the loss of HLA-A, HLA-C, and HLA-DRB1 alleles (see Table 4).

TABLE 67
HLA typing using Luminex.

| | Allele | |
|---|---|---|
| HLA | SL4-C | SL4-T |
| A | 01:11 | Not detected |
| B | 03:15 | Altered |
| C | 08:12 | Not detected |
| DRB1 | 03:12 | Not detected |
| DQB1 | 01:08 | Altered |

Finally, we assessed their interaction with HLA-DR1 using flow cytometry (refer to FIG. 6). It was found that SL4-T cells exhibited a lower level of binding with anti-HLA-DR antibodies.

### The Use of Treated Cells for the Transplantation of Bone Marrow.

To analyze the applicability of treatment of non-immune cells, we examined donor bone marrow to determine if we could modulate the interaction between HLA antigens and anti-HLA antibodies on stem cells. For this purpose, the bone marrow from a volunteer was treated with a combination of DNase and RNase (1 μg/mL) or with anti-DNA and anti-RNA antibodies.

HLA typing of the treated bone marrow was then performed using next-generation sequencing (refer to Table 5).

TABLE 68
Effect of tunning on bone marrow cells.

| | | |
|---|---|---|
| Gene | Allele 1 | Allele 2 |

Control (before treatment)

| | | |
|---|---|---|
| HLA-A | *01:01 | *02:01 |
| HLA-B | *08:01 | *44:02 |
| HLA-C | *07:01 | *07:02 |
| HLA-DRB1 | *03:01 | *15:01 |
| HLA-DQA1 | *01:01 | *05:01 |
| HLA-DQB1 | *02:01 | *06:02 |

Treated cells (after DNAse and RNase treatment)

| | | |
|---|---|---|
| HLA-A | *01:01 | Not determined |
| HLA-B | Not determined | Not determined |
| HLA-C | *07:01 | Not determined |
| HLA-DRB1 | *03:01 | Altered |
| HLA-DQA1 | Not determined | Not determined |
| HLA-DQB1 | Not determined | Not determined |

Treated cells (after anti-DNA and anti-RNA treatment)

| | | |
|---|---|---|
| HLA-A | *01:01 | Not determined |
| HLA-B | Not determined | Not determined |
| HLA-C | *07:01 | Not determined |
| HLA-DRB1 | *03:01 | Altered |
| HLA-DQA1 | Not determined | Not determined |
| HLA-DQB1 | Not determined | Not determined |

We next analyzed the effect of treatment on cells derived from various tissue types, including connective, epithelial, muscle, and nervous tissues. For treatment, we either applied continuous or multiple cycles of nuclease treatment, or utilized ant-DNA and anti-RNase antibodies. We focused on a total of five different HLA loci, examining two alleles at each locus. The cell cultures were sourced either from patient donors or purchased. These cells were cultivated following general recommendations for cell culture. The results of this analysis are presented in Table 6.

TABLE 69
The effect of treatment on the interaction
of HLA proteins with the antibodies.

| | | Alterations in the interaction of | | |
|---|---|---|---|---|
| | | at least one allele with antibodies | | |
| Cell | Type | HLA-A | HLA-B | HLA-C |
| Microglia Cells | Control | — | — | — |
| | Treated | Altered | Altered | Altered |
| chondrocytes | Control | — | — | — |
| | Treated | Altered | Altered | Altered |
| Primary Human Skeletal | Control | — | — | — |
| Muscle Cells | Treated | Altered | Altered | Altered |
| Primary Human | Control | — | — | — |
| Osteoblasts | Treated | Altered | Altered | Altered |
| Epithelial Cells | Control | — | — | — |
| | Treated | Altered | Altered | Altered |
| Adipocytes | Control | — | — | — |
| | Treated | Altered | Altered | Altered |
| Aortic Smooth Muscle | Control | — | — | — |
| Cells | Treated | Altered | Altered | Altered |
| Human kidney cells | Control | — | — | — |
| | Treated | Altered | Altered | Altered |
| Hepatic cells | Control | — | — | — |
| | Treated | Altered | Altered | Altered |
| Primary Pancreatic | Control | — | — | — |
| Epithelial Cells | Treated | Altered | Altered | Altered |

“—” - not altered

### Example 49. The Use of Treated Cells to Produce Universal Blood Components

Blood specimens of healthy volunteers with known A, B, 0, AB and Rh positive groups were obtained. The RBCs were leukodepleted and 7.0 log 10 cells per ml were resuspended in PBS (pH 7.2), harvested by centrifugation 600×g for 5 min, washed twice with PBS buffer, and resuspended in PBS buffer. For the treatment of RBCs, erythrocytes were harvested by centrifugation at 600×g for 5 min and treated with multiple times with RNase or anti-RNA antibodies to develop treated cells. The blood group performed by Column agglutination technology (CAT) using fully automated Immunohematological Equipment Autovue Innova (Ortho Clinical Diagnostics, USA) according to the manufacturer's instructions.

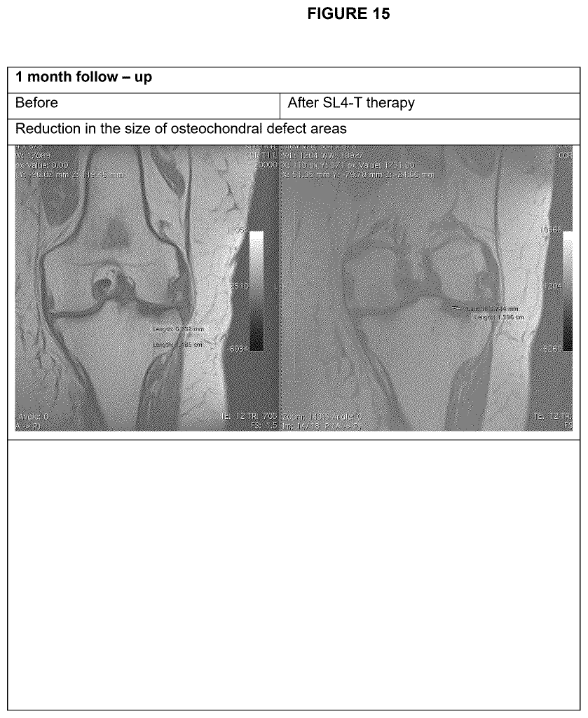

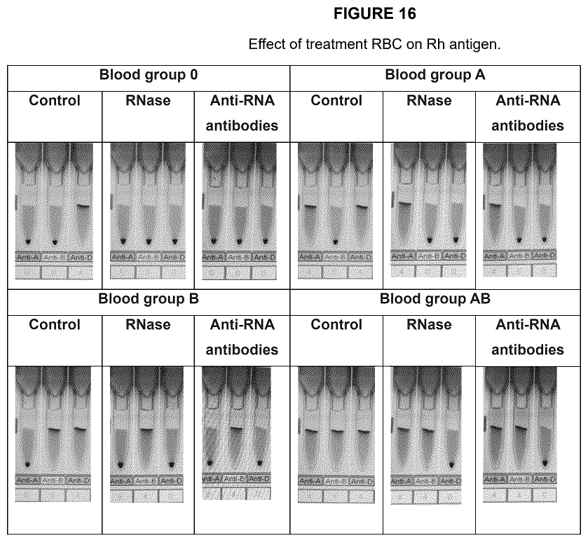

We found that the treatment of RBS results in the conversion of RhD positive to RhD negative (FIG. 1). This conversion was stable and was not restored up to seven days after the last treatment which was the last day, we measured it. Notably, the effect of conversion from Rh-positive to Rh-negative phenotype was observed in all four ABO blood groups following the treatment with either nucleases or ant-RNA antibodies. (FIG. 15).

We found that RhD positive erythrocytes treated with anti-exRNA antibodies exhibiting characteristics of RhD-negative RBCs and this effect was consistent across any of the ABO groups.

### Example 50. The Elimination of HLA Antigens from the Organs and Generation of Universal Donor Organs

Next, we investigated whether the interaction between MHC I and HLA antigens with antibodies could be prevented within mammalian organs. For this, we used liver tissue specimens from excess surgical material obtained during liver transplants. The tissues were freshly frozen and processed promptly for perfusion. We employed isolated liver perfusion, a method involving the perfusion of a specific portion or slice of the liver to model the whole organ, focusing particularly on parenchymal organ perfusion. Liver slices were obtained using a Krumdieck slicer or a similar instrument, set at a speed of 30, to produce slices thinner than 400 μm. These slices were placed into six-well plates filled with 3.5 mL of culture medium supplemented with DNase and RNase at concentrations of either 10 mg/L or 75 mg/L. The slices were then incubated at +37° C. in a 5% CO2 atmosphere with sufficient humidity, and the medium was replaced every 12 hours. Subsequently, the tissues were homogenized, and the interaction of HLA with relevant antibodies was analyzed. The results are presented in Table 70 below.

TABLE 70
Alteration of MHC antigens and their
interaction with the antibodies.

Alterations in the interaction of
at least one allele with antibodies

| Cell | HLA-A | HLA-B | HLA-C |
|---|---|---|---|
| Control | − | − | − |
| Treated with nucleases | + | + | − |
| at 10 mg/L | | | |
| Treated with nucleases | + | + | + |
| at 25 mg/L | | | |
| Treated with anti-DNA | + | + | + |
| and anti-RNA antibodies | | | |

“−” not altered;
“+” - altered

The data received indicate that the proposed method can be utilized to remove HLA specificity, enabling the generation of universal donor organs and tissues. Additionally, this method is applicable in organ printing, organ-on-chip technology, and the creation of unique primary cell cultures.

### Example 51. The Use of Treated Cells for the Treatment of Epithelial Defects

The objective of this research was to evaluate the in vivo activity of treated cells in a diabetic ulcer mouse model for delayed wound healing.

To study the effect of allogeneic cells, we used human-derived fibroblasts, and for the autologous effects, we used fibroblasts derived from the same mice. To obtain autologous primary fibroblasts, mice were anesthetized with a Ketamine/Xylazine mix. A small piece of tail was cut using sterile scissors and further divided into pieces smaller than 2 mm. These pieces were transferred into 2.0 ml tubes containing collagenase D-pronase solution. The tubes were shaken at 200 r/min for 180 minutes. Next, a 70 μm cell strainer was placed over cell culture dishes, and complete medium was added. The digested tail tissues were placed in the strainer and ground using a 10 ml syringe plunger for 10 minutes. The resulting cell suspension was then transferred to 15 ml tubes containing pre-warmed medium. Treated cells were obtained as previously described, by modifying their activity following repeated treatment with DNase and RNase.

Experiments were conducted in 16 diabetic female mice (db/db; BKS.Cg-Dock7m+/+Leprdb/J) 12 weeks of age. The mice were anesthetized with ketamine (0.13 mg/g, IP)+xylazine (20 mg/ml) and shaved to expose their back. A circular, full-thickness wound 0.6×0.6 cm patch of skin was then excised in a dorsal skin and bandaged with dressing for 3 days.

### Groups and Treatments:

5 days post-infection, mice were randomly assigned to one of the treatment groups (see, Table below).

TABLE 71
Effect of different therapies on wound healing.

Wound size (% of original wound size)

| | Fibroblast | Fibroblast | Fibroblast | Fibroblast |
|---|---|---|---|---|
| | control | control | treated | treated |
| Day of the | allogeneic | autologous | allogeneic | autologous |
| therapy | (human) | (mice) | (human) | (mice) |
| 0 | 100 | 100 | 100 | 100 |
| 5 | 96 ± 9 | 91 ± 13 | 52 ± 24* | 42 ± 17* |
| 10 | 114 ± 23 | 87 ± 14 | 10 ± 8* | 5 ± 3* |
| 15 | 91 ± 19 | 93 ± 16 | 0* | 0* |

*p < 0.05

The groups treated with treated autologous and allogeneic cells were able to accelerate wound healing, which can be used for the treatment of different epithelial defects including burns and ulcers.

### Example 52: Products and Method for Allogeneic Mesotherapy

A patient experiencing hairline decline was previously unsuccessfully treated with multiple rounds of autologous PRP therapy. Following the failure of the PRP therapy to enhance hair growth, the patient underwent two cycles of PRP therapy, utilizing allogeneic SL4-T. The results, as measured by hair count per 1 cm\hat{ }2, are presented in Table 72.

TABLE 72
The use of allogeneic treated cells
for the restoration for the hairline.

| Group | | Hair count |
|---|---|---|
| PRP | Baseline (before PRP | 100% |
| treatment | treatment) | |
| | After PRP treatment course | 104% |
| | 6 months after PRP treatment | 82% |
| | course | |
| SL4-T | Baseline (SL4-T therapy) | 79% (to baseline |
| therapy | | before PRP therapy) |
| | After SL4-T treatment course | 235%* |
| | 6 months after PRP treatment | 297%* |
| | course | |

*p < 0.05

The data received demonstrate that the use of SL4-T significantly facilitated hair growth.

### Example 53. The Use of Treated Microbial Cells for Microbiota Transplantation

We aimed to determine whether treatment the microbial mix from the donor could enhance their potential to engraft in the recipient. Microbiota engraftment was assessed by comparing the overall similarity between the donor's inoculum and the recipients' microbiota.

Male C57BL/6J specific pathogen-free 8-week-old mice were utilized. Feces were collected from donor mice in the morning, stored in sterile containers, and then diluted (1:20) in RPMI-1640. The samples were homogenized by vortex and treated with multiple rounds of DNase and RNase I at 20 mg/L for 5 or 16 cycles. These treated samples were used as inoculum.

For fecal microbiota transplantation, recipient mice were administered broad-spectrum antibiotics (neomycin 200 mg/kg, metronidazole 200 mg/kg, ampicillin 200 mg/kg, and vancomycin 100 mg/kg) for 5 days. Colonization with microbiota was achieved through intragastric gavage with 200 μl of inoculum once a day for three consecutive days.

Fecal samples were collected 28 days after the microbiota transplantation and stored at −80° C. until further processing. Bacterial DNA was extracted from the recipient's fecal samples and from the original recipients' feces using a QIAamp stool DNA mini kit following the manufacturer's instructions. Sequencing libraries of the V3-V4 region were prepared according to the Illumina MiSeq system instructions. In brief, the V3 and V4 regions of the 16S bacterial rRNA gene were amplified using a two-step polymerase chain reaction (PCR) protocol with V3 and V4 region primers (forward: 5′-TCGTCGGCAGCGTCAGATGTGTATAAGAGACAGCCTACGGGNGGCWGCAG-3′; reverse: 5′-GTCTCGTGGGCTCGGAGATGTGTATAAGAGACAGGACTACHVGGGTATCTAATCC-3′) for the first PCR and Nextera XT index primers for the second PCR. Amplicons were cleaned using AMPure XP magnetic beads. Illumina sequencing adapters and dual-index barcodes were then added to each amplicon. Libraries were assessed with the Qubit dsDNA HS assay kit and TapeStation high sensitivity D1000 ScreenTape. They were normalized and sequenced on an Illumina MiSeq instrument using a MiSeq reagent kit v2 (500 cycles). The data were analyzed using the MiSeq Reporter software Metagenomics workflow by Illumina, and the Bray-Curtis similarity between pairs of inoculum and donor samples was assessed. This analysis particularly focused on the Gammaproteobacteria, Bacillales, Enterobacterales, Bacteroidales, Lactobacilloae, Ruminococcaceae, Alcaligenaceae, Lachnospiraceae, Coriobacteriaceae, Gastranaerophilales, Prevotellaceae families, as well as Verrucomicrobiales, Erysipelotrichales, and Clostridiales. (FIG. 1).

### Example 54. The Use of Cell Treatment to Generate Cells with the Higher Secretion Capacity

We utilized mucus secreted by goblet cells as a reference model for modulating cell secretion function. HT29-MTX cells, derived from human colon carcinoma and known for mucin secretion, were cultivated in plastic culture flasks using RPMI-1640 medium within a 5% CO2 atmosphere in a humidified incubator. These cells were treated with multiple rounds of DNase and RNase, as described previously, to produce treated cells. Mucin-like glycoprotein secretion was quantified using an enzyme-linked lectin assay (ELLA) and expressed in nanograms per 10\hat{ }5 cells.

TABLE 73
Enhanced Cell Secretion Activity and Mucin
A Secretion Following Cell Treatment.

| HT-29 Control | HT-29 Treated |
|---|---|
| 103 ± 10.8 | 294.4 ± 51* |

*p < 0.05

The data received indicate that treatment cells can enhance their secretion activity, having higher reprogramming potential, promoting increased mucin secretion and the production of protective mucus layers.

### Example 55. The Use of Treatment of Immune Cells to Prevent Autoimmunity

We investigated whether cell treatment could be utilized to reset immunological memory and reprogram cells. Peripheral blood mononuclear cells (PBMCs) were isolated from blood samples of patients diagnosed with systemic lupus erythematosus (SLE) using Ficoll-Paque Plus density-gradient centrifugation. The lymphocyte-enriched fraction was washed with PBS (pH 7.2) and centrifuged at 500 g for 20 minutes. To generate treated B-cells, a portion of the samples underwent multiple treatments with DNase and RNase (B-T), while controls were treated with PBS (B-Mock). Additionally, some untreated B-cells were mixed with SL4-T cells in a 10:1 proportion. The viability of cells was monitored by MTT according to manufacturer's recommendations (Sigma).

The cells, at a density of 5×10\hat{ }5 cells per well in 96-well plates, were cultured in RPMI 1640. Antibody production was stimulated by adding 2.5 μg/ml CpG (InvivoGen), known to promote antibody secretion in B cells through TLR9 stimulation. The identification of antinuclear antibodies was conducted on day 5 using an ELISA immunoassay using SLE ELISA KIT (Table 1).

The groups were organized as follows: Group 1: Control B-cells/plasma cells (B-C) not stimulated; Group 2: Control B-cells/plasma cells (B-Mock) stimulated with CpG; Group 3: Treated B-cells/plasma cells (B-T) stimulated with CpG: Group 4: Untreated B-cells/plasma cells+SL4-T (B+SL4-T) stimulated with CpG.

TABLE 74
Analysis of autoantibody production.

| Probe | Antibody level |
|---|---|
| Group 1 | − |
| Group 2 | +++ |
| Group 3 | − |
| Group 4 | − |

The data we obtained indicate that control B-cells were involved in the production of antinuclear antibodies. In contrast, treated B-cells or the addition of SL4-T cells to B-cells suppressed autoantibody production.

### Example 56. The Use of Treated Cells for Interspecies Transplantation

Methods: Blood samples were obtained from horses, cows, pigs, and humans, using heparin-containing vacutainers (‘green’ tops) as an anticoagulant. These samples were stored at +4° C. until use. For each sample, 10 mL of blood was centrifuged at 1500×g for 5 minutes, and the plasma fraction was separated from the samples. The sediment was washed with the same volume of 0.9% NaCl, mixed, and the tubes were inverted. This centrifugation and washing process was repeated five times. After the final wash, 5 mL of PBS was added, and the resulting samples were diluted to a concentration of approximately ˜5×10\hat{ }8 red blood cells/mL. 1% Triton-X100 and PBS were used as positive and negative controls, respectively.

After adding the samples to wells, the plate was gently shaken for 20 seconds and incubated at room temperature for 30 minutes (sufficient for agglutination due to both antigens and immune reactions attacking foreign red blood cells). The contents of the wells were then centrifuged at 1500×g for 5 minutes. The supernatant was collected, transferred to a plate, and the optical density of each well was measured at 405 nm.

C-control cells; T-treated cells with multiple rounds of DNase and RNase treatment.

Table Title: Hemolytic activity and transplantability of treated cells between different unrelated organisms. (Table 75 below).

TABLE 75

| Agent | Horse | Cow | Pig | Human |
|---|---|---|---|---|
| Triton X-100 \| | 100 ± | 100 ± | 100 ± | 100 ± |
| positive control | 5 | 2.3 | 2.9 | 2 |

1:10

| | | | | |
|---|---|---|---|---|
| human WBC (C) | 58.9** ± | 58.4* ± | 1.7*** ± | 0.2** ± |
| | 5.8 | 6 | 1.9 | 1.8 |
| human WBC (T) | 2.2** ± | −0.2*** ± | −0.4** ± | −1.2*** ± |
| | 0.4 | 0.7 | 1 | 1.3 |
| horce WBC (C) | 3.3** ± | 38.5** ± | 41** ± | 36.9* ± |
| | 0.5 | 4.1 | 3.8 | 5.5 |
| horce WBC (T) | 1.7** ± | 2** ± | 0.4** ± | −1.8*** ± |
| | 1.6 | 0.8 | 0.8 | 0.1 |
| Cow WBC (C) | 60.2 ± | 0.9*** ± | −0.7** ± | −0.8*** ± |
| | 5.5 | 1 | 1.3 | 1.3 |
| Cow WBC (T) | 0.2** ± | 0.3*** ± | −1.7** ± | 0** ± |
| | 2.2 | 1.1 | 0.5 | 2.1 |
| Pig WBC (C) | 41.7* ± | 37.8** ± | −0.2*** ± | 41.2** ± |
| | 5.2 | 1.7 | 1.8 | 2 |
| Pig WBC (T) | 1.5** ± | −1.1*** ± | 0.5** ± | −0.4** ± |
| | 1.3 | 1.7 | 1.2 | 1 |

### Example 57. Use of Cell Treatment to Produce Programmable Protein-Producing Cells from Stem Cells

Human iPSCs derived from fibroblasts were cultured in low oxygen conditions using RPMI 1640 or Essential 8 medium. They were divided into groups: untreated controls, DNase-treated, RNase-treated, and those treated with both DNase and RNase. After differentiation into insulin-producing cells, a subset of controls was also treated with DNase and RNase, forming a fifth group. Insulin levels were measured with ELISA after incubating cells in glucose-supplemented medium. Results showed the impact of nuclease treatment on insulin production.

Results of the effect of treatment on insulin production are shown in the Table below.

TABLE 76
Insulin synthesis by developed insulin producing cells.

| | Cultivated | Cultivated | Cultivated in | Treated with |
|---|---|---|---|---|
| | in the | in the | the presence | multiple rounds |
| | presence | presence | of DNase and | of DNase and |
| Control | of DNase | of RNase | RNase | RNase |
| 1025 ± 96 | 1816 ± 229 | 2067 ± 187 | 2575 ± 453 | 2837 ± 499 |
| 95.60335 | 229.2248 | 187.6593 | 453.2549 | 499.796 |

Insulin secretion in response to 2.5 mM glucose. Results were normalized to the total protein concentration in supernatants and presented as picograms of insulin per milligram of the total protein content in supernatant of insulin producing cells.

To test in vivo insulin secretion, iPSCs were transplanted into diabetic STZ-induced Rag2−/−yc−/−mice.

The fasting blood glucose levels in these mice were monitored daily.

TABLE 77
Effect of Transplantation of Treated Cells on Glucose.

| | | Cells Treated with |
|---|---|---|
| | | multiple rounds of DNase |
| Day | Control | and RNase |
| 0 | 523 ± 0 | 500 ± 24 |
| 30 | 149 ± 34 | 471 ± 12 |

*p < 0.05

### Example 59: Products and Method for Allogeneic Mesotherapy

A patient experiencing hairline decline was previously unsuccessfully treated with multiple rounds of autologous PRP therapy. Following the failure of the PRP therapy to enhance hair growth, the patient underwent two cycles of PRP therapy, utilizing allogeneic SL4-T. The results, as measured by hair count per 1 cm\hat{ }2, are presented in Table 78.

TABLE 78
The use of allogeneic treated cells
for the restoration for the hairline.

| Group | | Hair count |
|---|---|---|
| PRP | Baseline (before PRP treatment) | 100% |
| treatment | After PRP treatment course | 104% |
| | 6 months after PRP treatment | 82% |
| | course | |
| SL4-T | Baseline (SL4-T therapy) | 79 (to baseline |
| therapy | | before PRP therapy) |
| | After SL4-T treatment course | 235%* |
| | 6 months after PRP treatment | 297%* |
| | course | |

*p < 0.05

### Example 60: The Use of Cell-Surface-Bound for the Development of an Anti-Aging Vaccine

Known DNA and RNA vaccines contain the DNA or RNA sequence that encodes the protein antigen, which is synthesized and against which an immune response is sought. In our case, antibodies are produced against the DNA or RNA itself.

Female/male SWISS mice were used utilized in this study. Upon initiation of the study, these mice were aged between 3 and 80 weeks. They were accommodated in standard plastic cages designed for mice, with each cage housing 5 to 6 animals. All mice were given unrestricted access to tap water and a standard diet (Purina 5L79, Rat and Mouse 18% protein, supplied by PMI Nutrition International, Brentwood, MO, USA).

Vaccination methods: Antigen (60 μg/mice) cell-surface-bound nucleic acids of Pseudomonas aeruginosa VT20 (VL-5P) or Escherichia coli VT 224 (VL-5E) or human white blood cells (VL-5H) was diluted in 100 μl PBS and mixed equally with Complete Freund's adjuvant (or AlOH3). Mice were injected intraperitoneally with 100 μl of this mix and subcutaneously at base of tail with 100 μl. A booster injection, consisting of 60 μg antigen, diluted in PBS, and mixed 1:1 with Incomplete Freund's adjuvant (or AlOH3) was administered after 3 weeks. One more booster injection was given either with 60 μg antigen in PBS 1:1.

### Major Signs:

Urinary Incontinence Test: Mice without water for 4 hours; filter papers analyzed under UV light for voids, measured with Image J. Home Cage Activity: Locomotor activity over 6 days, recording distance traveled in standard cages. Open Field Test: Anxiety assessed in a divided arena for 6 minutes, noting periphery time and immobility. Elevated Plus Maze: Exploration of open vs. closed arms over 10 minutes. Barnes Maze: Escape latency and errors in finding an escape box. Forced Swim Test: Behavioral responses in water-filled cylinders. Grip Strength Test: Maximum strength measured. Blood and Serum Analysis: CBC and serum tests for metabolic and organ function using ABX Pentra and Vitros analyzers.

### Mice Immunized at 35 Weeks/8 Months.

TABLE 79
Major signs | Absolute data.
Mean ± SD | Absolute data

| | 24 w | 48 w | 96 w | | | |
|---|---|---|---|---|---|---|
| Major sign | Control | Control | Control | VP-5P | VP-5E | VP-5H |

Urinary incontinence test

| | | | | | | |
|---|---|---|---|---|---|---|
| Number of | 8.3 ± 1.2 | 18 ± 2.2 | 22 ± 2.9 | 9 ± 1.1*** | 12 ± 3.0** | 20.7 ± 2.8 |
| PVS, n | | | | | | |
| Total urine, | 0.9 ± 0.13 | 1.9 ± 0.2 | 2.3 ± 0.3 | 0.9 ± 0.1*** | 1.3 ± 0.2** | 2.0 ± 0.3 |
| ml | | | | | | |

Home cage

| | | | | | | |
|---|---|---|---|---|---|---|
| Total | 1334 ± 76.9 | 1112 ± 134.6 | 620.9 ± 53.6 | 1164 ± 69.4*** | 983 ± 132.4* | 729 ± 96.5* |
| distance | | | | | | |
| traveled, | | | | | | |
| cm (10 min) | | | | | | |
| Open field | | | | | | |
| Distance | 846 ± 37.7 | 508.3 ± 3.8 | 336.2 ± 25.9 | 636.4 ± 76.1*** | 499.2 ± 38.7** | 424.2 ± 56.2** |
| traveled in | | | | | | |
| 6 minutes, | | | | | | |
| cm | | | | | | |

Elevated maze test

| | | | | | | |
|---|---|---|---|---|---|---|
| Open arms, | 63.8 ± 12.1 | 59.1 ± 9.8 | 53.8 ± 10 | 78.5 ± 5.4* | 61.0 ± 6.3 | 62.4 ± 5.1* |
| s | | | | | | |
| Transitions, | 15.4 ± 4 | 15.5 ± 4.9 | 17.7 ± 4.5 | 10.3 ± 5.2* | 15.5 ± 3.6 | 13.1 ± 4.0 |
| number | | | | | | |

Barnes maze Latency to escape, s

| | | | | | | |
|---|---|---|---|---|---|---|
| Day 1 | 57.7 ± 7.3 | 79.8 ± 10.7 | 136.1 ± 14.4 | 79.5 ± 8.4*** | 105.4 ± 12.2*** | 112.0 ± 10.6 |
| Day 4 | 38.8 ± 6.2 | 38.1 ± 8.4 | 81.9 ± 8.6 | 37.3 ± 5.8*** | 55.9 ± 5.9*** | 60.1 ± 6.2** |

Forced - swim test

| | | | | | | |
|---|---|---|---|---|---|---|
| Immobile | 26.3 ± 9.5 | 34.2 ± 8.8 | 48.3 ± 13.3 | 30.1 ± 11* | 38.0 ± 5.9* | 43.5 ± 5.2* |
| time, s | | | | | | |

Grip strength

| | | | | | | |
|---|---|---|---|---|---|---|
| Grip | 397 ± 11 | 387 ± 18 | 239 ± 15 | 374 ± 13 | 309 ± 15** | 290 ± 32 |
| strength, g | | | | | | |

Kidney function

| | | | | | | |
|---|---|---|---|---|---|---|
| Albumin, | 12 ± 3.1 | 111 ± 4.3 | 125 ± 2.6 | 26 ± 2.1*** | 54 ± 6.4*** | 82 ± 6.9* |
| mg/dl | | | | | | |

Full blood panel

| | | | | | | |
|---|---|---|---|---|---|---|
| WBC, | 9.5 ± 1 | 6.3 ± 0.1 | 6.9 ± 0.9 | 9.9 ± 0.8*** | 7.2 ± 0.5* | 7.1 ± 0.2* |
| 10³/mm³ | | | | | | |
| PLT, | 567 ± 83 | 435 ± 108 | 327 ± 60 | 644 ± 138** | 421 ± 103 | 405 ± 94 |
| 10³/mm³ | | | | | | |

Glucose

| | | | | | | |
|---|---|---|---|---|---|---|
| Glucose, | 5.6 ± 1.6 | 7.8 ± 1.7 | 9.0 ± 1.6 | 5.6 ± 1.1* | 6.3 ± 1.4* | 7.9 ± 1.1* |
| mmol/L | | | | | | |

*p < 0.05,
**p < 0.01,
***p < 0.001 when compared to the control mice of the same age (96 weeks)

TABLE 80
Kaplan - Meier study for VP-5P.

| Parameter | Control | VP-5P |
|---|---|---|
| Median survival | 61.5 | 116.5 |

| | |
|---|---|
| Ratio \| reciprocal | 1.894 |
| 95% CI of ratio | 0.357 to 0.781 |
| Mantel-Haenszel* (Hazard ratio) | 6.582 |

We used a booster vaccine for VP-5P which was given 6 and 12 month after the initial vaccination.

TABLE 81
Major signs | Absolute data.
Mean ± SEM | Absolute data

| | | | | | 72 w | 96 w | 96 weeks |
|---|---|---|---|---|---|---|---|
| | 24 w | 72 w | 72 w | 96 w | VP-5P + 1 | VP-5P + 1 | VP-5P + 2 |
| Major sign | Control | Control | VP-5P | VP-5P | booster | booster | boosters |
| Number of | 8 ± 0.5 | 25 ± 0.6 | 10 ± 0.5** | 16 ± 0.4** | 8 ± 0.3*** | 14 ± 0.3* | 14 ± 0.4** |
| PVS, n | | | | | | | |
| Total urine | 0.9 ± 0.3 | 2.6 ± 0.3 | 1.0 ± 0.3*** | 1.7 ± 0.2*** | 0.8 ± 0.04*** | 1.5 ± 0.04*** | 1.5 ± 0.05*** |
| volume, ml | | | | | | | |

Home cage

| | | | | | | | |
|---|---|---|---|---|---|---|---|
| Total | 1183 ± 109 | 689 ± 49 | 873 ± 102*** | 780 ± 75*** | 1029 ± 84*** | 926 ± 96*** | 920 ± 50** |
| distance | | | | | | | |
| traveled, | | | | | | | |
| cm (10 m) | | | | | | | |

Open field

| | | | | | | | |
|---|---|---|---|---|---|---|---|
| Distance | 884 ± 70 | 607 ± 53 | 895 ± 49*** | 862 ± 111*** | 905 ± 67*** | 910 ± 109***/*** | 1012 ± 98***/*** |
| traveled in | | | | | | | |
| 6 minutes, | | | | | | | |
| cm | | | | | | | |

Forced - swim test

| | | | | | | | |
|---|---|---|---|---|---|---|---|
| Immobile | 16 ± 0 | 29 ± 1 | 17 ± 1 | 23 ± 0 | 14 ± 0.8 | 17 ± 0.2 | 15 ± 0.4 |
| time, s | | | | | | | |

Kidney function*

| | | | | | | | |
|---|---|---|---|---|---|---|---|
| Albumin, | 36 ± 0.3 | 113 ± 1.6 | 76 ± 1.2*** | 103 ± 0.71** | 40 ± 0.97*** | 78 ± 0.3***/*** | 75 ± 0.71***/*** |
| mg/dl | | | | | | | |

Glucose*

| | | | | | | | |
|---|---|---|---|---|---|---|---|
| Glucose, | 4.8 ± 0.08 | 9.1 ± 0.21 | 5.8 ± 0.34*** | 5.9 ± 0.06** | 5.4 ± 0.16***/* | 5.1 ± 0.03***/* | 5 ± 0.07***/*** |
| mmol/L | | | | | | | |

*p < 0.05,
**p < 0.01,
***p < 0.001 when compared to the control mice of the same age
/***when compared with vaccinated animals that did not receive a booster

### Example 61

### Effect of Cells Tuning on Longevity

We examined the impact of cell tuning on longevity by assessing telomere length in white blood cells as an example.

White blood cells (WBC) were isolated from donor blood using the quadruple bag system.

Characteristics of blood donors are presented in Table 82 below.

TABLE 82

| Characteristics | #1 | #2 | #3 |
|---|---|---|---|
| Samples | blood | blood | blood |
| Sex | XY | XY | XX |
| Age, y | 22 | 26 | 21 |
| Height, cm | 187 | 191 | 179 |
| Weight, kg | 79 | 93 | 87 |
| Current smoker | no | no | no |
| Systolic blood pressure, mm Hg | 125 | 130 | 115 |
| Diastolic blood pressure, mm Hg | 75 | 80 | 75 |

To generate “tuned” WBCs (SL4-T) were treated with either DNase I and RNase A (SL4-Tn) or with anti-DNA and anti-RNA antibodies (SL4-Ta), as previously described. As a control we used untreated cells.

DNA from WBCs was isolated using a Qiagen DNA Midi kit. For PCR, we followed the protocols outlined by Lin J, Smith D L, Esteves K, Drury S in ‘Telomere length measurement by qPCR-Summary of critical factors and recommendations for assay design’ (Psychoneuroendocrinology, 2019 Jan. 1; 99:271-8) and used the following primers:

SEQ ID NO. 7
telg ACACTAAGGTTTGGGTTTGGGTTTG GGTTTGGGTTAGTGT
telc TGTTAGGTATCCCTATCCCTATCCCTATCCCTATCCCTAACA

### Probes:

The results of tuning cell telomere length are depicted in Table 83 below.

TABLE 83
The impact of cell tuning on telomere length.

| Untreated | SL4-Tn | SL4-Ta |
|---|---|---|
| 13.2 ± 0.4 | 16.7 ± 0.9* | 15.7 ± 1.2* |

*p < 0.05

### Example 62. The Use of Vaccine Together with Tuned Cells for the Increase of Longevity

The study aimed at evaluating the effectiveness of the vaccination with extracellular DNA of P. aeruginosa (eDNAPA) vaccination in conjunction with various adjuvants and additional agents reveals significant differences in the condition of mice, especially when compared to a control group. The primary vaccination agent, eDNAPA, combined with additional agents, including SL-4C, FB-C(control fibroblasts), SL4-T, and FB-T (fibroblasts tuned following multiple treatments with anti-DNA and anti-RNA antibodies), demonstrates improvements across a range of health-related parameters.

Individually, the eDNAPA vaccination improved the condition compared to the control group, reducing the actual age of mice in comparison to their physiological state, reflected in the decrease of the Frailty index. This improvement was particularly noticeable in aspects related to anxiety and activity under normal and stressful conditions, depression, muscle function, and laboratory data. The introduction of SL-4T and FB-T, both separately and in combination, without vaccination also shows positive effects, albeit to a lesser extent than when combined with the vaccine.

However, the most significant improvements were observed when the vaccination was combined with the processed forms SL4-T and FB-T. These combinations led to an improvement in bladder function, a reduction in anxiety, an improvement in activity under various conditions, a lower level of depression, and an improvement in muscle function. Laboratory data also reflected positive changes, indicating an improvement in overall health.

Interestingly, the combination of vaccination with processed forms SL4-T and FB-T not only improved the condition according to individual parameters but also contributed overall to the reduction of the Frailty index, indicating a slowdown in the aging processes and an improvement in the quality of life of the mice.

The eDNAPA vaccination significantly extends the lifespan of mice, and this effect can be further enhanced by combining the vaccine with additional agents such as SL4-T and FB-T. The combination of eDNAPA20 with both SL4-T and FB-T provides the most significant increase in estimated median survival as shown in Table 84 below.

TABLE 84

| | | | Corresponding Condition [weeks] | | | | | |
|---|---|---|---|---|---|---|---|---|
| | | | | Anxiety and | | | | |
| | | Actual | | Activity in | | | | |
| | | Age of | | Normal/ | | | | Median |
| Vaccination | Additional | Mice | Bladder | stressful | | Muscle | Laboratory | survival |
| Agent | Agents | [weeks] | Function | Conditions | Depression | Function | Data | [week] |
| Control | | 72 | 72 | 72 | 72 | 72 | 72 | 80 |
| eDNA_{PA20} | | 72 | 32 | 40 | 28 | 32 | 45 | 121 |
| — | SL-4C | 72 | 65 | 48 | 40 | 72 | 48 | 84 |
| — | FB-C | 72 | 50 | 50 | 72 | 72 | 48 | 81 |
| — | SL-4C + | 72 | 48 | 48 | 60 | 72 | 48 | 86 |
| | FB-C | | | | | | | |
| eDNA_{PA20} | SL-4C | 72 | 30 | 30 | 32 | 32 | 32 | 120* |
| eDNA_{PA20} | FB-C | 72 | 30 | 30 | 28 | 32 | 32 | 123* |
| eDNA_{PA20} | SL-4C + | 72 | 40* | 26* | 24* | 32* | 32* | 126* |
| | FB-C | | | | | | | |
| — | SL4-T | 72 | 45 | 48 | 38* | 72 | 45* | 98* |
| — | FB-T | 72 | 80 | 65 | 72 | 72 | 48* | 96* |
| — | SL4-T + | 72 | 26* | 48* | 60 | 72 | 80* | 106* |
| | FB-T | | | | | | | |
| eDNA_{PA20} | SL4-T | 72 | 30* | 30* | 28* | 32* | 32* | 125* |
| eDNA_{PA20} | FB-T | 72 | 35* | 30* | 28* | 32* | 32* | 128* |
| eDNA_{PA20} | SL4-T + | 72 | 30* | 26* | 28* | 32* | 32* | 130* |
| | FB-T | | | | | | | |

*p < 0.05

### Example 63. Effect of Treated Cells on Antimicrobial and Anticancer Activity of White Blood Cells

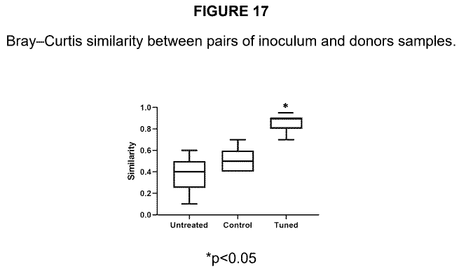

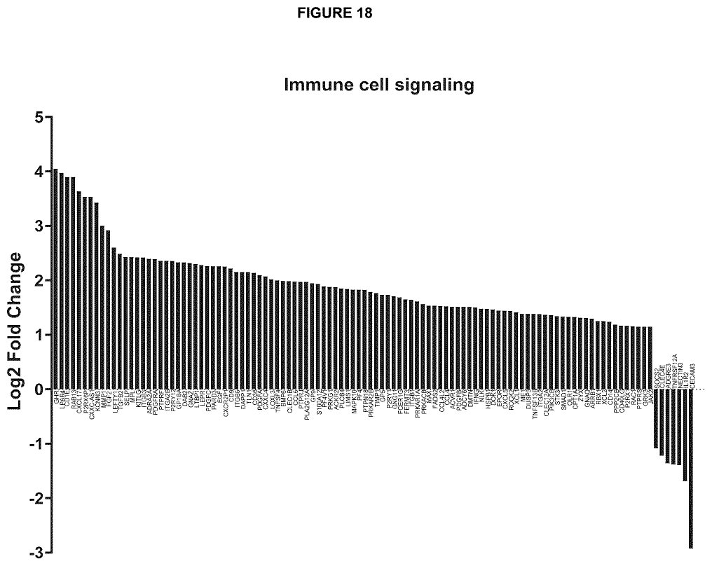

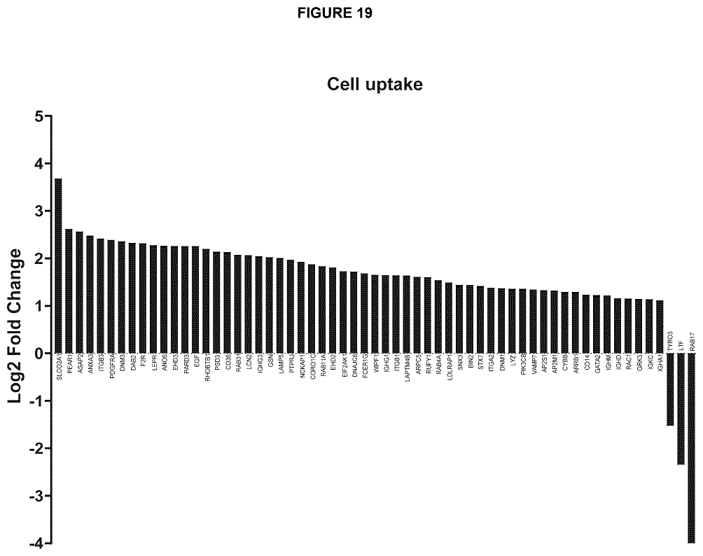

Our analysis revealed differential expression of numerous genes SL4-T compared to SL4-C. Our analysis revealed a positive enrichment of genes associated with numerous pathways including Immune cell signaling, Immune cell migration, and Cellular uptake. For instance, key pathways enriched included Chemokine signaling, Cytokine signaling, and MAPK signaling, transedothelial migration, extravasation, anaerobic energy pathways, energy metabolism including glyscolysis, actin cytoskeleton remodeling, endocytosis and phagocytosis pathways. The results of the differentially expressed genes profiles of SL4-T compared to SL4-C are shown in FIGS. 17, 18 and 19 below.

### Example 63. Effect of Treated Cells on Generation of Evolutionary Old Ancestor Organisms

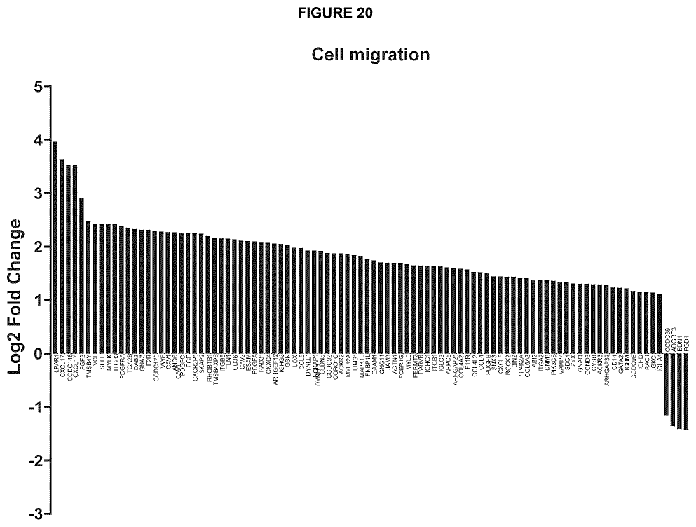

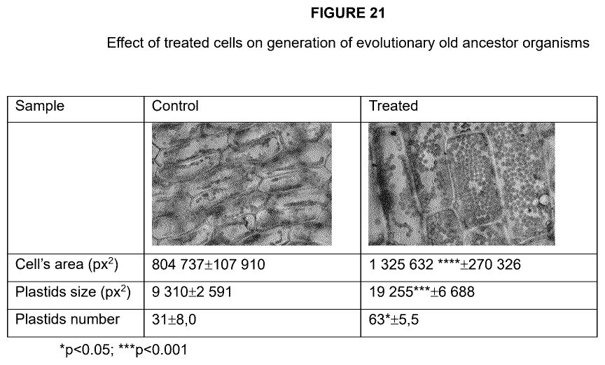

In order to get the evolutionary old organisms, we treated cells of Elodea with several rounds of nucleases. The resulted plant was able to generate the energy using evolutionary old pathways including anaerobic and to grow in darkness. The characteristics of the plants grown from the treated cells are shown in FIG. 20.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter. Further, the use of the terms “include,” “includes” and “including” means include, includes and or including as well as include, includes and including, but not limited to.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only and is not intended to limit the scope of the present invention, which is defined solely by the claims. Accordingly, the present invention is not limited to that precisely as shown and described.

Although embodiments of the current disclosure have been described comprehensively in considerable detail to cover the possible aspects, those skilled in the art would recognize that other versions of the disclosure are also possible.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit of this invention.

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

What is claimed is:

1. A method for treating an infection, wherein
a. a cell, which is not a chimeric antigen receptor T-cell, is treated with one or more of an RNase, a DNase, an enzyme that has DNase and/or RNase activity, an antibody that binds to an RNA and an antibody that binds to a DNA;
b. following treatment, a therapeutically effective amount of the treated cells are administered to a patient suffering from an infection; and
c. following administration, the treated cells reduce the symptoms of the infection.

2. The method of claim 1, wherein the infection is a bacterial, viral, fungal, protozoan or parasitic infection.

3. The method of claim 1, wherein the patient is suffering from a chronic infection, a temporary infection, a repeated infection or an acute infection.

4. The method of claim 1, wherein the cells are used to treat a respiratory infection.

5. The method of claim 1, wherein the cells are used to treat an infection comprising two or more of a bacteria, a virus, a fungus, a protozoa or a parasite.

6. The method of claim 1, wherein the treated cells produce one or more bioproducts that are capable of treating an infection in the patient.

7. The method of claim 6, wherein the one or more bioproducts reduce the severity of the infection

8. The method of claim 6, wherein the one or more bioproducts produced by the treated cells increase the survival time of the patient suffering from an infection as compared to a patient that is not administered the treated cells that produce one or more bioproducts.

9. The method of claim 1, wherein the administered treated cells are a combination of leukocytes and platelets.

10. The method of claim 1, wherein the treated cells are selected from an immune cell or a cellular derivative.

11. The treated cells of claim 10, wherein the cellular derivative is a platelet, an apoptotic body, an extracellular vesicle, a cell fragment and a bioproduct.

12. The immune cells of claim 10, which are selected from B-cells, T-cells, dendritic cells, macrophages, natural killer cells, neutrophils, monocytes, eosinophils, monocytes, basophils, plasma cells, CD34+ cells, cells of microglia and mast cells.

13. The method of claim 1, wherein the treated cells are autologous or allogeneic.

14. The method of claim 1, wherein the treated cells are treated two or more times with one or more of an RNase, a DNase, an antibody that binds to an RNA, an antibody that binds to a DNA and an enzyme with DNase or RNase activity.

15. The method of claim 1, wherein the enzyme that has DNase and/or RNase activity is benzonase.

16. The method of claim 14, wherein the two or more treatments increase the effectiveness of the treated cells to treat an infection.

17. The method of claim 1, wherein the treated cells are administered to a patient by intracerebral, intracerebroventricular, intraparenchymal injections, intrastriatal, intraspinal, parenteral, subcutaneous, intramuscular, intravenous, intraarterial, inhalation, intradermal, intrathecal, intracisterna magna, epidural and infusion, subarachnoid injection, enteral, oral, intramuscular, intrapleural, transdermal, rectal, nasal, buccal, sublingual, vaginal, intraperitoneal, topical, transdermal administration or inside the site of an infection.

18. A method of treating an infection, wherein,
a. a cell, which is not a chimeric antigen receptor T cell, is first treated with at least one of an RNase, a DNase, an enzyme that has DNase and/or RNase activity, an antibody that binds to an RNA and an antibody that binds to a DNA
b. following the first treatment, the cells receive a second treatment with a bacteria, a virus, a fungus, a protozoa, or a parasite; wherein,
c. following the two treatments, the ability of the treated cells to treat an infection is greater than cells that receive only the first but not the second treatment.

19. The method of claim 18, wherein the treated cells are first treated with products derived from Pseudomonas aeruginosa, E. coli, Staphylococcus aureus, Aspergillus spp., bacterial nucleic acids, LPS, dead Klebsiella pneumonia or active Klebsiella pneumonia.

20. The method of claim 18, wherein the cell is a cellular derivative, and further wherein, wherein the cellular derivative is a platelet, an apoptotic body, an extracellular vesicle, a cell fragment and a bioproduct.


SEQUENCE LISTING (5 sequences)

SEQ ID NO: 1 - protein, 662 aa, Homo sapien
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKQIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 662 aa, Homo sapien
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 672 aa, Homo sapien
QVQLVESGGGVVQPGRSLRLSCAASGFTFSSFGMHWVRQAPGKGLEWVAVISFDGSIKYSVDSVKGRFTISRDNSKNTLFLQMNSLRAEDTAVYYCARDRLNYYDSSGYYHYKYYGMAVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKQSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 4 - protein, 658 aa, Homo sapien
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 1328 aa, Homo sapien
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKQVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC